(19) United States
(12) Patent Application Publication
(10) Pub. No.: US 2023/0095836 A1
(43) Pub. Date: Mar. 30, 2023

(54) SUBSTITUTED 5,6-DIPHENYL-3(2H)-PYRIDAZINONES FOR USE AS FUNGICIDES

(71) Applicant: FMC Corporation, Philadelphia, PA (US)

(72) Inventors: Jeffrey Keith LONG, Wilmington, DE (US); Liana HIE, Wilmington, DE (US)

(21) Appl. No.: 17/798,956

(22) PCT Filed: Feb. 12, 2021
(86) PCT No.: PCT/US2021/017897

§ 371 Date: Aug. 11, 2022

Related U.S. Application Data
(60) Provisional application No. 62/976,573, filed on Feb. 14, 2020.

(51) Int. Cl. A01N 43/58; C07D 237/16; C07D 237/24; C07D 237/22; A01P 3/00

(52) U.S. Cl. CPC A01N 43/58; C07D 237/16; C07D 237/24; C07D 237/22; A01P 3/00

(57) ABSTRACT

Disclosed are compounds of Formula (1) including all geometric and stereoisomers, N-oxides, and salts thereof, wherein W, R¹, R², R³, R⁴, R⁵ m, n and p are as defined in the disclosure. Also disclosed are compositions containing the compounds of Formula (1) and methods for controlling plant disease caused by a fungal pathogen comprising applying an effective amount of a compound or a composition of the invention.

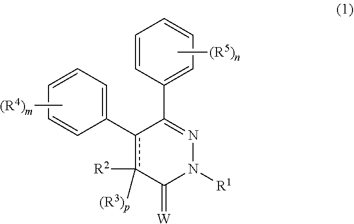

### FIELD OF THE INVENTION

This invention relates to certain pyridazinones, their N-oxides, salts and compositions, and methods of using them as fungicides.

### BACKGROUND OF THE INVENTION

The control of plant diseases caused by fungal plant pathogens is extremely important in achieving high crop efficiency. Plant disease damage to ornamental, vegetable, field, cereal and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publication WO 1982/00402 discloses diphenylpyridazinones and their use as herbicides and plant growth regulators.

European Patent Publication EP 478195(A1) discloses fungicidal dihydropyridazinones and pyridazinones and their use in agriculture.

U.S. Pat. No. 6,680,316 discloses pyridazin-3-ones and their use as pharmaceuticals.

U.S. Patent Publication US 2002/0123496 discloses pyridazine derivatives and their use as pharmaceuticals.

U.S. Patent Publication US 2007/0021418 discloses a method of inhibiting the production of osteopontin comprising administering a pyridazine derivative.

### SUMMARY OF THE INVENTION

This invention is directed to compounds of Formula 1 (including all stereoisomers), N-oxides, and salts thereof, compositions containing them and their use as fungicides:

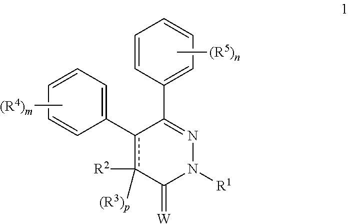

wherein
W is O or S;
R¹ is C₁-C₆ alkyl, C₂-C₆ alkenyl, C₂-C₆ alkynyl, C₃-C₆ cycloalkyl, C₂-C₆ cyanoalkyl or C₂-C₆ alkoxyalkyl, each optionally substituted with up to 3 substituents independently selected from halogen;
R² is H, halogen, cyano, hydroxy, nitro, C(═O)NR^{7a}R^{7b}, C(═O)OH, C₁-C₆ alkyl, C₁-C₆ haloalkyl, C₂-C₆ alkenyl C₂-C₆ haloalkenyl, C₂-C₆ alkynyl, C₂-C₆ haloalkynyl, C₃-C₆ cycloalkyl, C₃-C₆ halocycloalkyl, C₂-C₆ cyanoalkyl, C₁-C₆ hydroxyalkyl, C₂-C₆ alkoxyalkyl, C₂-C₆ haloalkoxyalkyl, C₁-C₆ alkoxy, C₁-C₆ haloalkoxy, C₂-C₆ alkenyloxy, C₂-C₆ haloalkenyloxy, C₂-C₆ alkynyloxy, C₂-C₆ haloalkynyloxy, C₃-C₆ cycloalkoxy, C₂-C₆ alkylcarbonyloxy, C₂-C₆ haloalkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₂-C₆ alkylcarbonyl, C₂-C₆ haloalkylcarbonyl, C₂-C₆ alkoxycarbonyl, C₂-C₆ haloalkoxycarbonyl, C₁-C₆ alkylamino, C₁-C₆ haloalkylamino or C₂-C₆ dialkylamino;
p is 0 or 1;
the dotted line in Formula 1 represents an optional bond, provided that the optional bond is present when p is 0, and the optional bond is absent when p is 1;
R³ is H or C₁-C₃ alkyl;
each R⁴ and R⁵ is independently cyano, nitro, halogen or hydroxy; or C₁-C₆ alkyl, C₂-C₆ alkenyl, C₂-C₆ alkynyl, C₂-C₆ cyanoalkyl, C₁-C₆ hydroxyalkyl, C₁-C₆ alkoxy, C₂-C₆ alkenyloxy, C₂-C₆ alkynyloxy, C₂-C₆ cyanoalkoxy or C₁-C₆ alkylthio, each optionally substituted with up to 3 substituents independently selected from halogen and C₁-C₃ alkyl; or —U—V-T;
each U is independently a direct bond, O, C(═O) or NR⁶;
each V is independently C₁-C₆ alkylene, C₂-C₆ alkenylene or C₃-C₆ alkynylene, wherein up to 2 carbon atoms are C(═O), each optionally substituted with up to 3 substituents independently selected from halogen, cyano, nitro, hydroxy, C₁-C₆ alkyl, C₁-C₆ haloalkyl, C₁-C₆ alkoxy and C₁-C₆ haloalkoxy;
each T is independently NR^{7a}R^{7b}, OR⁸ or S(═O)_{q}R⁹;
each R⁶ is independently H, C₁-C₆ alkyl, C₁-C₆ haloalkyl, C₂-C₆ alkylcarbonyl, C₂-C₆ haloalkylcarbonyl, C₂-C₆ alkoxycarbonyl, C₂-C₆ (alkylthio)carbonyl or C₂-C₆ alkoxy(thiocarbonyl);
each R^{7a} and R^{7b} is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₁-C₆ alkoxy, C₁-C₆ haloalkoxy, C₂-C₆ alkylcarbonyl or C₂-C₆ alkoxycarbonyl; or
R^{7a} and R^{7b} are taken together with the nitrogen atom to which they are attached to form a 3- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹⁰;
each R⁸ and R⁹ is independently H, C₁-C₆ alkyl, C₁-C₆ haloalkyl, C₂-C₆ alkenyl, C₂-C₆ haloalkenyl, C₂-C₆ alkynyl, C₃-C₆ cycloalkyl, C₃-C₆ halocycloalkyl, C₂-C₆ alkylcarbonyl, C₂-C₆ haloalkylcarbonyl or C₂-C₆ alkoxycarbonyl;
each R¹⁰ is independently halogen, C₁-C₃ alkyl, C₁-C₃ haloalkyl, C₁-C₃ alkoxy or C₁-C₃ haloalkoxy; and
m and n are each independently 0 to 5;
each q is independently 0, 1 or 2.
   provided that the compound of Formula 1 is not:
2-methyl-5,6-diphenyl-3(2H)-pyridazinone;
2-ethyl-5,6-diphenyl-3(2H)-pyridazinone;
2-(methoxymethyl)-5,6-diphenyl-3(2H)-pyridazinone;
2-(2-methoxyethyl)-5,6-diphenyl-3(2H)-pyridazinone;
2-(2-methoxyethyl)-5,6-diphenyl-3(2H)-pyridazinethione;
2,3-dihydro-2-methyl-3-oxo-5,6-diphenyl-4-pyridazinecarbonitrile;
2,3-dihydro-3-oxo-5,6-diphenyl-2-propyl-4-pyridazinecarbonitrile;
2,3-dihydro-2-(1-methylethyl)-3-oxo-5,6-diphenyl-4-pyridazinecarbonitrile;
5-cyano-6-oxo-3,4-diphenyl-1(6H)-pyridazinepropanenitrile;
2,3-dihydro-3-oxo-2-(2-pentyn-1-yl)-5,6-diphenyl-4-pyridazinecarbonitrile;
5,6-bis(4-chlorophenyl)-2-methyl-3(2H)-pyridazinone;
2-(methoxymethyl)-5-(4-methylphenyl)-6-phenyl-3 (2H)-pyridazinone;
5-(4-chlorophenyl)-2-(methoxymethyl)-6-phenyl-3 (2H)-pyridazinone;
2-(2-chloroethyl)-5,6-bis(4-chlorophenyl)-2,3-dihydro-3-oxo pyridazinecarbonitrile;
5,6-bis(4-methoxyphenyl)-2-methyl-3(2H)-pyridazinone;
2-ethyl-5,6-bis(4-methoxyphenyl)-3(2H)-pyridazinone;
5,6-bis(4-methoxyphenyl)-2-(1-methylethyl)-3(2H)-pyridazinone;
2-cyclopropyl-5,6-bis(4-methoxyphenyl)-3(2H)-pyridazinone;
2-(2-chloroethyl)-5,6-bis(4-methoxyphenyl)-3(2H)-pyridazinone;
5,6-bis(4-methoxyphenyl)-2-(2-propen-1-yl)-3(2H)-pyridazinone;
2-cyclopentyl-5,6-bis(4-methoxyphenyl)-3(2H)-pyridazinone;
2-ethyl-2,3-dihydro-5,6-bis(4-methoxyphenyl)-3-oxo-4-pyridazinecarbonitrile;
2,3-dihydro-5,6-bis(4-methoxyphenyl)-3-oxo-2-propyl-4-pyridazinecarbonitrile;
2,3-dihydro-5,6-bis(4-methoxyphenyl)-2-(1-methylethyl)-3-oxo-4-pyridazinecarbonitrile;
2-ethyl-6-(3-fluoro-4-methoxyphenyl)-5-(4-methoxyphenyl)-3(2H)-pyridazinone;
2-ethyl-5-(3-fluoro-4-methoxyphenyl)-6-(4-methoxyphenyl)-3(2H)-pyridazinone;
2-ethyl-5,6-bis(3-fluoro-4-methoxyphenyl)-3(2H)-pyridazinone;
2-ethyl-5,6-bis(3-fluoro-4-methoxyphenyl)-4,5-dihydro-3(2H)-pyridazinone;
6-(4-methoxyphenyl)-2-methyl-5-(3,4,5-trimethoxyphenyl)-3(2H)-pyridazinone;
2-methyl-4-nitro-5,6-diphenyl-3(2H)-pyridazinone;
2-methyl-4-(methylthio)-5,6-diphenyl-3(2H)-pyridazinone; and
4-(ethylthio)-2-methyl-5,6-diphenyl-3(2H)-pyridazinone.

More particularly, this invention pertains to a compound of Formula 1 (including all stereoisomers), an N-oxide or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

This invention also relates to a composition comprising a compound of Formula 1, an N-oxide, or a salt thereof, and at least one invertebrate pest control compound or agent.

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “agronomic” refers to the production of field crops such as for food and fiber and includes the growth of maize or corn, soybeans and other legumes, rice, cereal (e.g., wheat, oats, barley, rye and rice), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (e.g., berries and cherries) and other specialty crops (e.g., canola, sunflower and olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

The term “crop vigor” refers to rate of growth or biomass accumulation of a crop plant. An “increase in vigor” refers to an increase in growth or biomass accumulation in a crop plant relative to an untreated control crop plant. The term “crop yield” refers to the return on crop material, in terms of both quantity and quality, obtained after harvesting a crop plant. An “increase in crop yield” refers to an increase in crop yield relative to an untreated control crop plant.

The term “biologically effective amount” refers to the amount of a biologically active compound (e.g., a compound of Formula 1) sufficient to produce the desired biological effect when applied to (i.e. contacted with) a fungus to be controlled or its environment, or to a plant, the seed from which the plant is grown, or the locus of the plant (e.g., growth medium) to protect the plant from injury by the fungal disease or for other desired effect (e.g., increasing plant vigor).

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating agent” does not limit the carbon-containing radical to alkyl; the carbon-containing radicals in alkylating agents include the variety of carbon-bound substituent radicals specified, for example, for R¹.

As referred to in this disclosure, the terms “fungal pathogen” and “fungal plant pathogen” include pathogens in the Ascomycota, Basidiomycota and Zygomycota phyla, and the fungal-like Oomycota class that are the causal agents of a broad spectrum of plant diseases of economic importance, affecting ornamental, turf, vegetable, field, cereal and fruit crops. In the context of this disclosure, “protecting a plant from disease” or “control of a plant disease” includes preventative action (interruption of the fungal cycle of infection, colonization, symptom development and spore production) and/or curative action (inhibition of colonization of plant host tissues).

As used herein, the term “mode of action” (MOA) is as define by the Fungicide Resistance Action Committee (FRAC), and is used to distinguish fungicides according to their biochemical mode of action in the biosynthetic pathways of plant pathogens, and their resistance risk. FRAC-defined modes of actions include (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis, (I) melanin synthesis in cell wall, (P) host plant defense induction, (U) unknown mode of action, (NC) not classified, (M) chemicals with multi-site contact activity and (BM) biologicals with multiple modes of action. Each mode of action (i.e. letters A through BM) contain one or more subgroups (e.g., A includes subgroups A1, A2, A3 and A4) based either on individual validated target sites of action, or in cases where the precise target site is unknown, based on cross resistance profiles within a group or in relation to other groups. Each of these subgroups (e.g., A1, A2, A3 and A4) is assigned a FRAC code (a number and/or letter). For example, the FRAC code for subgroup A1 is 4. Additional information on target sites and FRAC codes can be obtained from publicly available databases maintained, for example, by FRAC.

As used herein, the term “cross resistance” refers to the phenomenon that occurs when a pathogen develops resistance to one fungicide and simultaneously becomes resistant to one or more other fungicides. These other fungicides are typically, but not always, in the same chemical class or have the same target site of action, or can be detoxified by the same mechanism.

Generally, when a molecular fragment (i.e. radical) is denoted by a series of atom symbols (e.g., C, H, N, O and S) the implicit point or points of attachment will be easily recognized by those skilled in the art. In some instances herein, particularly when alternative points of attachment are possible, the point or points of attachment may be explicitly indicated by a hyphen (“-”). The dotted line in rings depicted in the present description (e.g., the ring in Formula 1) represents that the bond indicated can be a single bond or double bond.

In the above recitations, the term “alkyl”, used either alone or in compound words such as “haloalkyl” includes straight-chain and branched alkyl, such as, methyl, ethyl, n-propyl and i-propyl. “Alkenyl” includes straight-chain and branched alkenes such as ethenyl, 1-propenyl, 2-propenyl, and the different butenyl and pentenyl isomers. “Alkenyl” also includes polyenes such as 1,2-propadienyl and 2,4-pentadienyl. “Alkynyl” includes straight-chain and branched alkynes such as ethynyl, 1-propynyl, 2-propynyl, and the different butynyl and pentynyl isomers. “Alkynyl” can also include moieties comprised of multiple triple bonds such as 2,5-pentadiynyl.

“Alkoxy” includes, for example, methoxy, ethoxy, n-propyloxy, i-propyloxy, and the different butoxy isomers. “Alkenyloxy” includes straight-chain and branched alkenyl attached to and linked through an oxygen atom. Examples of “alkenyloxy” include H₂C═CHCH₂O and CH₃CH═CHCH₂O. “Alkynyloxy” includes straight-chain and branched alkynyl attached to and linked through an oxygen atom. Examples of “alkynyloxy” include HC═CCH₂O and CH₃C≡CCH₂O.

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂OCH₂ and CH₃CH₂CH₂OCH₂CH₂. “Alkoxyalkoxy” denotes alkoxy substitution on another alkoxy moiety. Examples of “alkoxyalkoxy” include CH₃OCH₂O, CH₃OCH₂CH₂CH₂O and CH₃CH₂OCH₂O.

The term “cycloalkyl” denotes a saturated carbocyclic ring consisting of between 3 to 5 carbon atoms linked to one another by single bonds. Examples of “cycloalkyl” include cyclopropyl, cyclobutyl and cyclopentyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include CF₃, ClCH₂, CF₃CH₂CH₂ and CF₃CCl₂.

“Cyanoalkyl” denotes an alkyl group substituted with one cyano group. Examples of “cyanoalkyl” include NCCH₂, NCCH₂CH₂ and CH₃CH(CN)CH₂. The term “cyanoalkoxy” is defined analogously to the term “cyanoalkyl”.

The total number of carbon atoms in a substituent group is indicated by the “Cᵢ-Cⱼ” prefix where i and j are numbers from 1 to 5. For example, C₁-C₃ alkyl designates methyl through propyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 3 substituents independently selected from halogen” means that 0, 1, 2 or 3 substituents can be present (if the number of potential connection points allows).

When a compound is substituted with a substituent bearing a subscript that indicates the number of said substituents can vary (e.g., (R⁴)ₘ in Formula 1 wherein m is 0 to 5), then said substituents are independently selected from the group of defined substituents, unless otherwise indicated. When a variable group is shown to be optionally attached to a position, for example (R⁴)ₘ wherein m may be 0, then hydrogen may be at the position even if not recited in the definition of the variable group.

Naming of substituents in the present disclosure uses recognized terminology providing conciseness in precisely conveying to those skilled in the art the chemical structure. For sake of conciseness, locant descriptors may be omitted.

The term “carbocyclic ring” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

As used herein, the term “partially unsaturated ring” or “partially unsaturated heterocycle” refers to a ring which contains unsaturated ring atoms and one or more double bonds but is not aromatic.

The terms “heterocyclic ring” or “heterocycle” denotes a ring wherein at least one of the atoms forming the ring backbone is other than carbon. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or aromatic heterocyclic ring. “Saturated heterocyclic ring” refers to a heterocyclic ring containing only single bonds between ring members.

Compounds of this invention can exist as one or more stereoisomers. Stereoisomers are isomers of identical constitution but differing in the arrangement of their atoms in space and include enantiomers, diastereomers, cis- and trans-isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

This invention comprises all stereoisomers, conformational isomers and mixtures thereof in all proportions as well as isotopic forms such as deuterated compounds.

One skilled in the art will appreciate that not all nitrogen containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides, and agriculturally suitable salts, and solvates thereof.

Compounds selected from Formula 1, stereoisomers, tautomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

Embodiments of the present invention as described in the Summary of the Invention include those described below. In the following Embodiments, Formula 1 includes stereoisomers, N-oxides, and salts thereof, and reference to “a compound of Formula 1” includes the definitions of substituents specified in the Summary of the Invention unless further defined in the Embodiments.
Embodiment 1. A compound of Formula 1 wherein W is O.
Embodiment 2. A compound of Formula 1 wherein W is S.
Embodiment 3. A compound of Formula 1 or Embodiments 1-2 wherein R¹ is C₁-C₃ alkyl, C₂-C₅ alkenyl, C₂-C₅ alkynyl, C₃-C₅ cycloalkyl, C₂-C₄ cyanoalkyl or C₂-C₅ alkoxyalkyl.
Embodiment 4. A compound of Embodiment 3 wherein R¹ is C₁-C₃ alkyl, C₂-C₃ alkenyl, C₂-C₃ alkynyl, C₃-C₄ cycloalkyl, C₂-C₃ cyanoalkyl or C₂-C₃ alkoxyalkyl.
Embodiment 5. A compound of Embodiment 4 wherein R¹ is C₁-C₂ alkyl, C₃-C₄ cycloalkyl or C₂-C₃ cyanoalkyl.
Embodiment 6. A compound of Embodiment 5 wherein R¹ is methyl, ethyl, cyclopropyl or —CH₂C≡N.
Embodiment 7. A compound of Embodiment 6 wherein R¹ is methyl, ethyl or cyclopropyl.
Embodiment 8. A compound of Embodiment 7 wherein R¹ is methyl or ethyl.
Embodiment 9. A compound of Embodiment 8 wherein R¹ is methyl.
Embodiment 10. A compound of Embodiment 8 wherein R¹ is ethyl.
Embodiment 11. A compound Formula 1 wherein R¹ is methyl, ethyl or cyclopropyl, each optionally substituted with up to 3 substituents independently selected from halogen.
Embodiment 12. A compound of Embodiment 11 wherein R¹ is methyl or halomethyl.
Embodiment 13. A compound of Formula 1 or anyone of Embodiments 1 through 12 wherein R² is H, halogen, cyano, C(═O)NR^{7a}R^{7b}, C₁-C₃ alkyl, C₁-C₃ haloalkyl, C₂-C₃ alkenyl C₂-C₃ haloalkenyl, C₂-C₃ alkynyl, C₂-C₃ haloalkynyl, C₃-C₅ cycloalkyl, C₃-C₅ halocycloalkyl, C₁-C₃ alkoxy, C₁-C₃ haloalkoxy, C₂-C₃ alkenyloxy, C₂-C₃ haloalkenyloxy, C₂-C₃ alkynyloxy, C₂-C₃ haloalkynyloxy, C₃-C₅ cycloalkoxy, C₁-C₃ alkylamino or C₂-C₃ dialkylamino.
Embodiment 14. A compound of Embodiment 13 wherein R² is H, halogen, cyano, C₁-C₃ alkyl, C₁-C₃ haloalkyl, cyclopropyl, halocyclopropyl, C₁-C₃ alkoxy, C₁-C₃ haloalkoxy, C₂-C₃ alkenyloxy or C₂-C₃ haloalkenyloxy.
Embodiment 15. A compound of Embodiment 14 wherein R² is H, halogen, cyano, C₁-C₃ alkyl, C₁-C₃ haloalkyl, cyclopropyl, C₁-C₃ alkoxy or C₁-C₃ haloalkoxy.
Embodiment 16. A compound of Embodiment 15 wherein R² is H, halogen, cyano, C₁-C₂ alkyl, C₁-C₂ haloalkyl, C₁-C₂ alkoxy or C₁-C₂ haloalkoxy.
Embodiment 17. A compound of Embodiment 16 wherein R² is H, halogen, C₁-C₂ alkyl, haloalkyl or methoxy.
Embodiment 18. A compound of Embodiment 17 wherein R² is H, halogen, C₁-C₂ alkyl or methoxy.
Embodiment 19. A compound of Embodiment 18 wherein R² is Br, Cl, methyl, ethyl or methoxy.
Embodiment 19a. A compound of Embodiment 19 wherein R² is C₁, methyl, ethyl or methoxy.
Embodiment 19b. A compound of Embodiment 19a wherein R² is Cl or methyl.
Embodiment 20. A compound of Embodiment 19 wherein R² is methyl.
Embodiment 21. A compound of Embodiment 13 wherein R² is H, halogen, cyano, methyl, ethyl, C₁-C₂ haloalkyl or cyclopropyl.
Embodiment 22. A compound of Embodiment 21 wherein R² is H, halogen, cyano, methyl, ethyl, halomethyl or cyclopropyl.
Embodiment 23. A compound of Embodiment 22 wherein R² is H, halogen, cyano, methyl, ethyl or cyclopropyl.
Embodiment 24. A compound of Embodiment 23 wherein R² is H, Br, C1 cyano or methyl.
Embodiment 25. A compound of Embodiment 24 wherein R² is H, Br, Cl, methyl or ethyl.
Embodiment 26. A compound of Embodiment 25 wherein R² is H, Br, Cl or methyl.
Embodiment 27. A compound of Formula 1 or anyone of Embodiments 1 through 26 wherein p is 0 (i.e. the optional bond in Formula 1 is present).
Embodiment 28. A compound of Formula 1 or anyone of Embodiments 1 through 26 wherein p is 1 (i.e. the optional bond in Formula 1 is absent).
Embodiment 29. A compound of Formula 1 or anyone of Embodiments 1 through 28 wherein R³ is H, methyl or ethyl.
Embodiment 30. A compound of Embodiment 29 wherein R³ is H or methyl.
Embodiment 31. A compound of Embodiment 30 wherein R³ is H.
Embodiment 32. A compound of Embodiment 30 wherein R³ is methyl.
Embodiment 33. A compound of Formula 1 or anyone of Embodiments 1 through 32 wherein each R⁴ and R⁵ is independently cyano, nitro or halogen; or C₁-C₃ alkyl, C₂-C₃ alkenyl, C₂-C₄ cyanoalkyl, C₁-C₃ alkoxy, C₂-C₄ alkenyloxy or C₂-C₄ cyanoalkoxy, each optionally substituted with up to 3 substituents independently selected from halogen; or —U—V-T.
Embodiment 34. A compound of Embodiment 33 wherein each R⁴ and R⁵ is independently cyano or halogen; or C₁-C₂ alkyl, C₂-C₃ cyanoalkyl, C₁-C₂ alkoxy, C₂-C₃ alkenyloxy or C₂-C₃ cyanoalkoxy, each optionally substituted with up to 3 substituents independently selected from halogen; or —U—V-T.
Embodiment 35. A compound of Embodiment 34 wherein each R⁴ and R⁵ is independently cyano or halogen; or C₁-C₂ alkyl or C₁-C₂ alkoxy, each optionally substituted with up to 3 substituents independently selected from halogen; or —U—V-T.
Embodiment 36. A compound of Embodiment 35 wherein each R⁴ and R⁵ is independently halogen or methoxy.
Embodiment 36a. A compound of Embodiment 36 wherein each R⁴ and R⁵ is independently Br, Cl, F or methoxy.
Embodiment 37. A compound of Formula 1 or anyone of Embodiments 1 through 36a wherein each R⁴ and R⁵ is independently cyano, nitro or halogen; or C₁-C₂ alkyl, C₂-C₄ alkenyl, C₂-C₄ alkynyl, C₁-C₃ alkoxy, C₂-C₄ alkenyloxy, C₂-C₄ alkynyloxy or C₂-C₄ cyanoalkoxy, each optionally substituted with up to 3 substituents independently selected from halogen.
Embodiment 38. A compound of Embodiment 37 wherein each R⁴ and R⁵ is independently cyano, nitro, halogen, C₁-C₂ alkyl, C₂-C₃ alkenyl, C₂-C₃ alkynyl, C₁-C₂ alkoxy, C₂-C₃ alkenyloxy, C₂-C₃ alkynyloxy or C₂-C₃ cyanoalkoxy.
Embodiment 39. A compound Embodiment 38 wherein each R⁴ and R⁵ is independently cyano, nitro, halogen, C₁-C₂ alkyl, C₂-C₃ alkenyl, C₁-C₂ alkoxy, C₂-C₃ alkenyloxy or C₂-C₃ cyanoalkoxy.
Embodiment 40. A compound Embodiment 39 wherein each R⁴ and R⁵ is independently cyano, nitro, halogen, C₁-C₂ alkyl, C₁-C₂ alkoxy, C₂-C₃ alkenyloxy or C₂-C₃ cyanoalkoxy.
Embodiment 41. A compound Embodiment 40 wherein each R⁴ and R⁵ is independently cyano, nitro, halogen, C₁-C₂ alkyl, C₁-C₂ alkoxy or C₂-C₃ cyanoalkoxy.
Embodiment 42. A compound Embodiment 41 wherein each R⁴ and R⁵ is independently cyano, nitro, Br, Cl, F, C₁-C₂ alkyl, C₁-C₂ alkoxy or C₂-C₃ cyanoalkoxy.
Embodiment 43. A compound Embodiment 42 wherein each R⁴ and R⁵ is independently cyano, nitro, Br, Cl, F, methyl or methoxy.
Embodiment 44. A compound Embodiment 43 wherein each R⁴ and R⁵ is independently nitro, Br, Cl, F or methoxy.
Embodiment 45. A compound of Embodiment 44 wherein each R⁴ is independently Cl, F or methoxy.
Embodiment 46. A compound Embodiment 45 wherein each R⁴ is independently Cl or F.
Embodiment 47. A compound Embodiment 46 wherein each R⁴ is F.
Embodiment 48. A compound Embodiment 44 wherein each R⁵ is independently Br, Cl, F or methoxy.
Embodiment 49. A compound Embodiment 48 wherein each R⁵ is independently Cl, F or methoxy.
Embodiment 50. A compound Embodiment 49 wherein each R⁵ is Cl or methoxy.
Embodiment 51. A compound of Formula 1 or any one of Embodiments 1 through 50 wherein each U is independently a direct bond, O, C(═O) or NH.
Embodiment 52. A compound of Embodiment 51 wherein each U is independently a direct bond, O or NH.
Embodiment 53. A compound of Embodiment 52 wherein each U is independently a direct bond or O.
Embodiment 54. A compound of Embodiment 53 wherein each U is independently a direct bond.
Embodiment 55. A compound of Embodiment 53 wherein each U is independently O.
Embodiment 56. A compound of Formula 1 or any one of Embodiments 1 through 55 wherein each V is independently C₁-C₃ alkylene, wherein up to 2 carbon atoms are C(═O), each optionally substituted with up to 3 substituents independently selected from halogen, C₁-C₂ alkyl, C₁-C₂ haloalkyl, C₁-C₂ alkoxy and C₁-C₂ haloalkoxy.
Embodiment 57. A compound of Embodiment 56 wherein each V is independently C₁-C₃ alkylene, wherein up to 1 carbon atom is C(═O), each optionally substituted with up to 2 substituents independently selected from halogen, methyl, halomethyl and methoxy.
Embodiment 58. A compound of Embodiment 57 wherein each V is independently CH₂, CH₂CH₂ or C(═O).
Embodiment 59. A compound of Embodiment 58 wherein each V is CH₂.
Embodiment 60. A compound of Formula 1 or any one of Embodiments 1 through 59 wherein each T is independently NR^{7a}R^{7b} or OR⁸.
Embodiment 61. A compound of Formula 1 or any one of Embodiments 1 through 60 wherein when R^{7a} and R^{7b} are separate (i.e. not taken together to form a ring), then each R^{7a} and R^{7b} is independently H, C₁-C₃ alkyl, C₁-C₃ haloalkyl, cyclopropyl, C₂-C₃ alkylcarbonyl or C₂-C₃ alkoxycarbonyl.
Embodiment 62. A compound of Embodiment 61 wherein each R^{7a} and R^{7b} is independently H, C₁-C₂ alkyl, C₁-C₂ haloalkyl or cyclopropyl.
Embodiment 63. A compound of Embodiment 62 wherein each R^{7a} and R^{7b} is independently H, methyl or halomethyl.
Embodiment 64. A compound of Formula 1 or any one of Embodiments 1 through 63 wherein each R⁸ and R⁹ is independently H, C₁-C₃ alkyl, C₁-C₃ haloalkyl, C₂-C₃ alkenyl, C₂-C₃ haloalkenyl or cyclopropyl.
Embodiment 65. A compound of Embodiment 64 wherein each R⁸ and R⁹ is independently H, C₁-C₂ alkyl or C₁-C₂ haloalkyl.
Embodiment 66. A compound of Embodiment 65 wherein each R⁸ and R⁹ is independently methyl or ethyl.
Embodiment 67. A compound of Formula 1 or any one of Embodiments 1 through 66 wherein each R¹⁰ is independently halogen, methyl, halomethyl or methoxy.
Embodiment 68. A compound of Formula 1 or any one of Embodiments 1 through 67 wherein each q is 0 or 2.
Embodiment 69. A compound of Formula 1 or any one of Embodiments 1 through 68 wherein m and n are each independently 1 to 5.
Embodiment 70. A compound of Embodiment 69 wherein m and n are each independently 1 to 4.
Embodiment 71. A compound of Embodiment 70 wherein m and n are each independently 1 to 3.
Embodiment 72. A compound of Embodiment 71 wherein m is 2 or 3.
Embodiment 73. A compound of Embodiment 72 wherein m is 2.
Embodiment 74. A compound of Embodiment 72 wherein m is 3.
Embodiment 75. A compound of Formula 1 or any one of Embodiments 1 through 76 wherein n is 1 to 4.
Embodiment 76. A compound of Embodiment 70 wherein n is 2 to 4.
Embodiment 77. A compound of Embodiment 76 wherein n is 2 or 3.
Embodiment 78. A compound of Embodiment 77 wherein n is 2.
Embodiment 79. A compound of Embodiment 77 wherein n is 3.
Embodiment 80. A compound of Formula 1 or any one of Embodiments 1 through 79 wherein m is 2 and n is 2 or 3.
Embodiment 81. A compound of Formula 1 or any one of Embodiments 1 through 82 wherein m is 2 and R⁴ is attached at the 2- and 6-positions (i.e. ortho positions); or m is 2 and R⁴ is attached at the 2- and 4-positions (i.e. ortho and para positions); or m is 2 and R⁴ is attached at the 3- and 5-positions (i.e. meta positions), all relative to the connection of the phenyl ring to the remainder of Formula 1.
Embodiment 82. A compound of Embodiment 81 wherein m is 2 and R⁴ is attached at the 2- and 6-positions (i.e. ortho positions); or m is 2 and R⁴ is attached at the 2- and 4-positions (i.e. ortho and para positions).
Embodiment 83. A compound of Embodiment 82 wherein m is 2 and R⁴ is attached at the 2- and 6-positions (i.e. ortho positions).
Embodiment 83a. A compound of Embodiment 82 wherein m is 2 and R⁴ is attached at the 2- and 4-positions (i.e. ortho positions).
Embodiment 84. A compound of Formula 1 or any one of Embodiments 1 through 83a wherein n is 2 and R⁵ is attached at the 3- and 5-positions (i.e. meta positions); or n is 2 and R⁵ is attached at the 2- and 4-positions (i.e. ortho and para positions); or n is 2 and R⁵ is attached at the 2- and 6-positions (i.e. ortho positions); or n is 3 and R⁵ is attached at the 2-, 3- and 5-positions (i.e. ortho and meta positions).
Embodiment 85. A compound of Embodiment 84 wherein n is 2 and R⁵ is attached at the 3- and 5-positions (i.e. meta positions); or n is 3 and R⁵ is attached at the 2-, 3- and 5-positions (i.e. ortho position and meta positions).
Embodiment 86. A compound of Embodiment 85 wherein n is 2 and R⁵ is attached at the 3- and 5-positions (i.e. meta positions).
Embodiment 87. A compound of Embodiment 86 wherein n is 3 and R⁵ is attached at the 2-, 3- and 5-positions (i.e. ortho position and meta positions).
Embodiment 88. A compound of Formula 1 or any one of Embodiments 1 through 87 wherein at least one of m or n is other than 0.
Embodiment 89. A compound of Formula 1 or any one of Embodiments 1 through 88 wherein when p is 0, then at least one of m or n is other than 0.
Embodiment 90. A compound of Formula 1 or any one of Embodiments 1 through 89 wherein at least one of m or n is other than 0, and at least one R⁴ or R⁵ is at an ortho position.
Embodiment 91. A compound of Formula 1 or any one of Embodiments 1 through 90 wherein when p is 0, then at least one of m or n is other than 0, and at least one R⁴ or R⁵ is at an ortho position.

Embodiments of this invention, including Embodiments 1-91 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-91 above as well as any other embodiments described herein, and any combination thereof, pertain to the compositions and methods of the present invention.

Combinations of Embodiments 1-91 are illustrated by:

Embodiment A. A compound of Formula 1 wherein
W is O;
R¹ is C₁-C₃ alkyl, C₂-C₅ alkenyl, C₂-C₅ alkynyl, C₃-C₅ cycloalkyl, C₂-C₄ cyanoalkyl or C₂-C₅ alkoxyalkyl;
R² is H, halogen, cyano, C₁-C₃ alkyl, C₁-C₃ haloalkyl, cyclopropyl, halocyclopropyl, C₁-C₃ alkoxy, C₁-C₃ haloalkoxy, C₂-C₃ alkenyloxy or C₂-C₃ haloalkenyloxy;
R³ is H, methyl or ethyl;
each R⁴ and R⁵ is independently cyano, nitro or halogen; or C₁-C₃ alkyl, C₂-C₃ alkenyl, C₂-C₄ cyanoalkyl, C₁-C₃ alkoxy, C₂-C₄ alkenyloxy or C₂-C₄ cyanoalkoxy, each optionally substituted with up to 3 substituents independently selected from halogen; or —U—V-T;
each U is independently a direct bond, O, C(═O) or NH;
each V is independently C₁-C₃ alkylene, wherein up to 1 carbon atom is C(═O), each optionally substituted with up to 2 substituents independently selected from halogen, methyl, halomethyl and methoxy;
each T is independently NR^{7a}R^{7b} or OR⁸;
each R^{7a} and R^{7b} is independently H, C₁-C₃ alkyl, C₁-C₃ haloalkyl, cyclopropyl, C₂-C₃ alkylcarbonyl or C₂-C₃ alkoxycarbonyl; and
m and n are each independently 1 to 4.
   Embodiment B. A compound of Embodiment A wherein
R¹ is C₁-C₂ alkyl, C₃-C₄ cycloalkyl or C₂-C₃ cyanoalkyl;
R² is H, halogen, cyano, C₁-C₂ alkyl, C₁-C₂ haloalkyl, C₁-C₂ alkoxy or C₁-C₂ haloalkoxy;
R³ is H or methyl;
each R⁴ and R⁵ is independently cyano or halogen; or C₁-C₂ alkyl or C₁-C₂ alkoxy, each optionally substituted with up to 3 substituents independently selected from halogen; or —U—V-T;
each U is independently a direct bond, O or NH;
each V is independently CH₂, CH₂CH₂ or C(═O);
each R^{7a} and R^{7b} is independently H, C₁-C₂ alkyl, C₁-C₂ haloalkyl or cyclopropyl;
m and n are each independently 1 to 3.
   Embodiment C. A compound of Embodiment B wherein
R¹ is methyl, ethyl, cyclopropyl or —CH₂CN;
R² is H, halogen, C₁-C₂ alkyl or methoxy;
p is 0; and
each R⁴ and R⁵ is independently halogen or methoxy.
   Embodiment D. A compound of Embodiment C wherein
R¹ is methyl;
R² is Br, Cl, methyl, ethyl or methoxy;
each R⁴ and R⁵ is independently Br, Cl, F or methoxy;
m is 2 and the R⁴ substituents are attached at the 2- and 6-positions; or m is 2 and the R⁴ substituents are attached at the 2- and 4-positions; or m is 2 and the R⁴ substituents are attached at the 3- and 5-positions; and
n is 2 and the R⁵ substituents are attached at the 3- and 5-positions; or n is 2 and the R⁵ substituents are attached at the 2- and 4-positions; or n is 2 and the R⁵ substituents are attached at the 2- and 5-positions; or n is 2 and the R⁵ substituents are attached at the 2- and 6-positions; or n is 3 and the R⁵ substituents are attached at the 2-, 3- and 5-positions.
   Embodiment E. A compound of Embodiment D wherein
R² is C₁, methyl, ethyl or methoxy;
each R⁴ is independently Cl or F; and
each R⁵ is independently Br, Cl, F or methoxy.
   Embodiment F. A compound of Embodiment E wherein
R² is Cl or methyl;
each R⁵ is independently Cl, F or methoxy.
   Embodiment G. A compound of anyone of Embodiments A through F wherein
m is 2; and
n is 2 or 3.

Specific embodiments include compounds of Formula 1 selected from the group consisting of:
6-(2-chloro-3,5-dimethoxyphenyl)-5-(2-chloro-4-fluorophenyl)-2-methyl-3(2H)-pyridazinone (Compound 25);
4-chloro-6-(2-chloro-3,5-dimethoxyphenyl)-5-(2,6-difluorophenyl)-2-methyl-3(2H)-pyridazinone (Compound 35);
5,6-bis(2-chloro-4-fluorophenyl)-2,4-dimethyl-3(2H)-pyridazinone (Compound 44);
4-chloro-6-(2-chloro-5-methoxyphenyl)-5-(2,6-difluorophenyl)-2-methyl-3(2H)-pyridazinone (Compound 57);
4-chloro-6-(2-chloro-3,5-dimethoxyphenyl)-5-(2-chloro-4-fluorophenyl)-2-methyl-3(2H)-pyridazinone (Compound 58);
6-(2-bromo-3,5-dimethoxyphenyl)-5-(2-chloro-4-fluorophenyl)-2,4-dimethyl-3(2H)-pyridazinone (Compound 64);
6-(2-chloro-3,5-dimethoxyphenyl)-5-(2-chloro-4-fluorophenyl)-2,4-dimethyl-3(2H)-pyridazinone (Compound 66);
6-(2-chloro-5-methoxyphenyl)-5-(2,6-difluorophenyl)-2,4-dimethyl-3(2H)-pyridazinone (Compound 68);
6-(2-bromo-3,5-dimethoxyphenyl)-4-chloro-5-(2-chloro-4-fluorophenyl)-2-methyl-3(2H)-pyridazinone (Compound 77);
6-(2-chloro-3,5-dimethoxyphenyl)-5-(2-chloro-4-fluorophenyl)-4-methoxy-2-methyl-3(2H)-pyridazinone (Compound 80);
4-chloro-5-(2-chloro-4-fluorophenyl)-6-(2-chloro-5-methoxyphenyl)-2-methyl-3(2H)-pyridazinone (Compound 83);
5-(2-chloro-4-fluorophenyl)-6-(2-chloro-5-methoxyphenyl)-2,4-dimethyl-3(2H)-pyridazinone (Compound 102);
5-(2-chloro-4-fluorophenyl)-6-(2-chloro-5-methoxyphenyl)-4-ethyl-2-methyl-3(2H)-pyridazinone (Compound 134);
6-(2-chloro-5-methoxyphenyl)-5-(2,6-difluorophenyl)-4-ethyl-2-methyl-3(2H)-pyridazinone (Compound 179); and
6-(2-chloro-3,5-dimethoxyphenyl)-5-(2,6-difluorophenyl)-2,4-dimethyl-3(2H)-pyridazinone (Compound 194).

Embodiments of this invention also including Embodiments AA through FF below.

Embodiment AA. A compound of Formula 1 wherein
W is O or S;
R¹ is C₁-C₃ alkyl, C₂-C₅ alkenyl, C₂-C₅ alkynyl, C₃-C₅ cycloalkyl, C₂-C₄ cyanoalkyl or C₂-C₅ alkoxyalkyl, each optionally substituted with up to 3 substituents independently selected from halogen;
R² is H, cyano, halogen, C₁-C₃ alkyl, C₁-C₃ haloalkyl or C₃-C₅ cycloalkyl;
the dotted line in Formula 1 represents an optional bond;
p is 0 or 1, provide that when the optional bond is present p is 0, and when the optional bond is absent p is 1;
R³ is H or C₁-C₃ alkyl;
each R⁴ and R⁵ is independently cyano, nitro or halogen; or C₁-C₃ alkyl, C₂-C₄ alkenyl, C₂-C₄ alkynyl, C₁-C₄ alkoxy, C₂-C₄ alkenyloxy, C₂-C₄ alkynyloxy, C₂-C₄ alkoxyalkoxy, C₂-C₄ alkoxyalkyl or C₂-C₄ cyanoalkoxy, each optionally substituted with up to 3 substituents independently selected from halogen and C₁-C₃ alkyl; and
m and n are each independently 0 to 5.
   Embodiment BB. A compound of Embodiment AA wherein
W is O;
R¹ is C₁-C₃ alkyl, C₂-C₅ alkenyl, C₂-C₅ alkynyl, C₃-C₅ cycloalkyl, C₂-C₄ cyanoalkyl or C₂-C₅ alkoxyalkyl;
R² is H, cyano, halogen, methyl, ethyl, halomethyl or cyclopropyl;
R³ is H, methyl or ethyl;
each R⁴ and R⁵ is independently cyano, nitro, halogen, C₁-C₂ alkyl, C₂-C₃ alkenyl, C₂-C₃ alkynyl, C₁-C₂ alkoxy, C₂-C₃ alkenyloxy, C₂-C₃ alkynyloxy, C₂-C₃ alkoxyalkoxy, C₂-C₃ alkoxyalkyl or C₂-C₃ cyanoalkoxy; and
m and n are each independently 1 to 4.
   Embodiment CC. A compound of Embodiment BB wherein
R¹ is C₁-C₂ alkyl, C₃-C₄ cycloalkyl or C₂-C₃ cyanoalkyl;
R² is H, cyano, halogen, methyl, ethyl or cyclopropyl;
R³ is H or methyl;
each R⁴ and R⁵ is independently cyano, nitro, halogen, C₁-C₂ alkyl, C₁-C₂ alkoxy or C₂-C₃ cyanoalkoxy;
m and n are each independently 1 to 3.
   Embodiment DD. A compound of Embodiment CC wherein
R¹ is methyl, ethyl, cyclopropyl or —CH₂C1\1;
R² is H, cyano, Br, Cl, methyl or ethyl;
p is 0; and
each R⁴ and R⁵ is independently is independently cyano, nitro, Br, Cl, F, C₁-C₂ alkyl, C₁-C₂ alkoxy or C₂-C₃ cyanoalkoxy.
   Embodiment EE. A compound of Embodiment DD wherein
R¹ is methyl, ethyl or cyclopropyl;
R² is H, cyano, Br, Cl or methyl;
each R⁴ and R⁵ is independently cyano, nitro, Br, Cl, F, methyl or methoxy; and
m is 2; and
n is 2 or 3.
   Embodiment FF. A compound of Embodiment EE wherein
R¹ is methyl or ethyl;
R² is H, Br, Cl or methyl;
each R⁴ is independently Cl or F; and
each R⁵ is independently nitro, Br, Cl, F or methoxy.

In addition to the embodiments described above, this invention also provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention also provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to a plant seed, a fungicidally effective amount of a compound of Formula 1 (including all stereoisomers, N-oxides, and salts thereof). Of note as embodiments of such methods are methods comprising applying a fungicidally effective amount of a compound corresponding to any of the compound embodiments described above. Of particular note are embodiments where the compounds are applied as compositions of this invention.

One or more of the following methods and variations as described in Schemes 1-10 can be used to prepare the compounds of Formula 1. The definitions of W, R¹, R², R³, R⁴, R⁵, m, n and p in the compounds of Formulae 1-11 below are as defined above in the Summary of the Invention unless otherwise noted. Compounds of Formulae 1a, 1b, 1c, 1d and 1e are various subsets of the compounds of Formula 1, and all substituents for Formulae 1a, 1b, 1c, 1d and 1e are as defined above for Formula 1 unless otherwise noted.

As shown in Scheme 1, compounds of Formula 1 can be prepared by alkylation of compounds of Formula 2 with a compound of the formula R¹-Lg wherein Lg is a leaving group such as halogen (e.g., Cl, Br) or sulfonate (e.g., methanesulfonate). Particularly useful alkylating agents include, but are not limited to, alkyl halides, and the like, (e.g., iodoethane, allyl bromide, propargyl chloride) and alkyl sulfates (e.g., dimethyl sulfate). Typically the reaction is run in the presence of a base such as sodium hydride, potassium tert-butoxide, sodium ethoxide or potassium carbonate, and in a solvent compatible with the base, such as dimethyl sulfoxide, N,N-dimethylformamide, tetrahydrofuran, acetonitrile or ethanol. The reaction can be carried out at temperatures ranging from about 0 to 100° C. For reaction conditions see Journal of Medicinal Chemistry 1980, 23(12), 1398-1405. Also, present Example 14, Step E illustrates the method of Scheme 1.

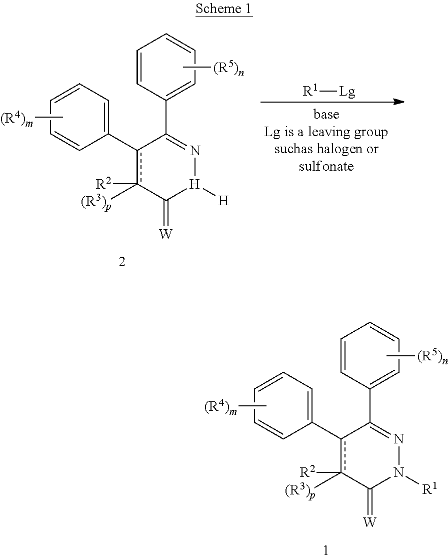

As shown in Scheme 2, compounds of Formula 1a (i.e. Formula 1 wherein the dotted line represents a bond and p is 0) can be prepared by oxidative dehydrogenation of corresponding compounds of Formula 1b (i.e. Formula 1 wherein the dotted line is not present and p is 1). A wide array of oxidizing agents and reaction conditions are suitable for the method of Scheme 2. For example, oxygen can be used as the oxidant in the presence of a copper (II) salt such as copper (II) chloride and a solvent such as acetonitrile (see, for example, Synthetic Communications 2000, 30(1), 1-7). Copper (II) acetate can also be used in the presence of a base such as sodium carbonate and a solvent such as toluene at a temperature between about ambient and the boiling point of the solvent (see, for example, European Journal of Organic Chemistry 2013, 2013(27), 6130-6136). Compounds of Formula 1b can also be treated with elemental halogen (e.g., Cl₂, Br₂, I₂) in a solvent such as acetic acid or dimethyl sulfoxide to provide compounds of Formula 1a. For relevant references using dihalides, see for example, Arzneimittel Forschung 2005, 55(6), 318-325 and Chinese Chemical Letters 2011, 22(12), 1435-1438. Alternatively, activated manganese dioxide can be used as the oxidizing agent in a solvent such as dichloromethane, dichloroethane, toluene or chlorobenzene, at a temperature between about ambient and the boiling point of the solvent. The reaction can also be carried out at temperatures above the solvent boiling point using a pressurized vessel, optionally with a microwave reactor. The method of Scheme 2 using manganese dioxide is illustrated in present Example 2.

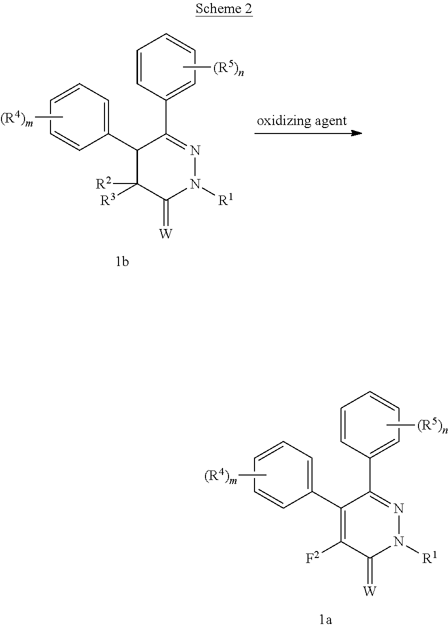

One skilled in the art will recognize that intermediate compounds of Formula 2 wherein the dotted line represents a bond and p is 0 can be prepared analogous to the oxidation method described in Scheme 2 above where R¹ is replaced by H.

As shown in Scheme 3, compounds of Formula 1b wherein W is 0 can be prepared by cyclization of α-keto acids or esters of Formula 3 with appropriately substituted hydrazines of Formula 4. The reaction can be run in a variety of solvents, such as ethanol, 1-butanol, tetrahydrofuran, 1,4-dioxane, heptane or toluene. In some cases, an acid or base catalyst can be added to the reaction mixture to promote elimination of water. Particularly useful catalysts include bases such as pyridine, sodium acetate or triethylamine; or acids such as acetic acid, oxalic acid or hydrochloric acid. Alternatively, an acid salt of Formula 4 hydrazines can be used in combination with a base such as an alkali metal hydroxide or carbonate, preferably sodium acetate. In some instances it may be advantageous to carry out the initial condensation of Formulae 3 and 4 in an alcoholic solvent (e.g., ethanol), concentrate the reaction mixture and then add a solvent such as toluene or chlorobenzene followed by heating under azeotropic conditions, optionally in the presence of an acid catalyst such as sulfuric acid or p-toluenesulfonic acid. Cyclization reactions of this type are well-documented in the chemical literature; see, for example, Archives of Pharmacal Research 2010, 33(1), 25-46; Journal of Medicinal Chemistry 2001, 44(16), 2511-2522; Monatshefte fuer Chemie 2004, 135(12), 1519-1527; and Indo Global Journal of Pharmaceutical Sciences 2016, 6(2), 65-71. Present Example 1, Step D illustrates the method of Scheme 3 using methylhydrazine to prepare a compound of Formula 1b wherein R¹ is methyl.

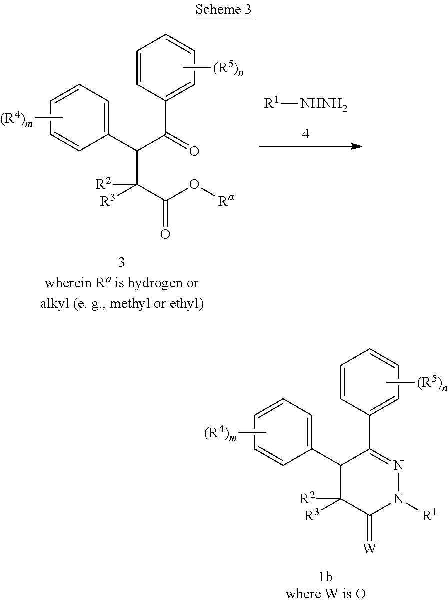

Intermediate compounds of Formula 2 wherein the dotted line is not present, and p is 1 can be prepared analogous to the method described in Scheme 3 using hydrazine or hydrazine hydrate in place of the compound of Formula 4.

As shown in Scheme 4, α-keto esters of Formula 3 wherein R^{a} is alkyl can be prepared by alkylation of diaryl ketones of Formula 5 with compounds of Formula 6 wherein Lg is a leaving group such as halogen (e.g., Cl, Br) or sulfonate (e.g., methanesulfonate) in the presence of a base such as sodium hydride, sodium acetate, potassium tert-butoxide, lithium diisopropylamide or lithium bis(trimethylsilyl)amide. The reaction is typically run in an appropriate solvent chosen for compatibility with the base such as dimethyl sulfoxide, N,N-dimethylformamide, tetrahydrofuran, 2-methyl-2-propanol or toluene. The use and choice of the appropriate solvent will be apparent to one skilled in chemical synthesis. For a representative procedure of the alkylation method of Scheme 4 using lithium diisopropylamide in tetrahydrofuran, see Journal of Medicinal Chemistry 2006, 49(2), 456-458. Also, the method of Scheme 4 using sodium hydride in a mixture of dimethyl sulfoxide and tetrahydrofuran is illustrated in Example 1, Step B.

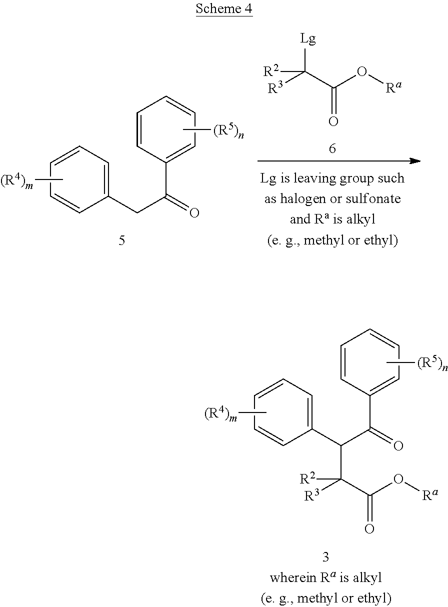

Carboxylic acids of Formula 3 wherein R^{a} is hydrogen can be readily prepared by hydrolysis of the corresponding esters wherein R^{a} is alkyl by means well-known to one skilled in the art. Present Example 1, Step C illustrates the hydrolysis of an ethyl ester of Formula 3 to the corresponding carboxylic acid.

Alternatively, as shown in Scheme 5, compounds of Formula 3 wherein R³ is H can be prepared by reaction of compounds of Formula 7 and benzaldehydes of Formula 8 in the presence of a cyanide salt such as sodium cyanide. For reaction conditions see, for example, Chemische Berichte 1976, 109(2), 541-545.

Compounds of Formulae 7 and 8 are commercially available and can be prepared by methods documented in the chemistry literature.

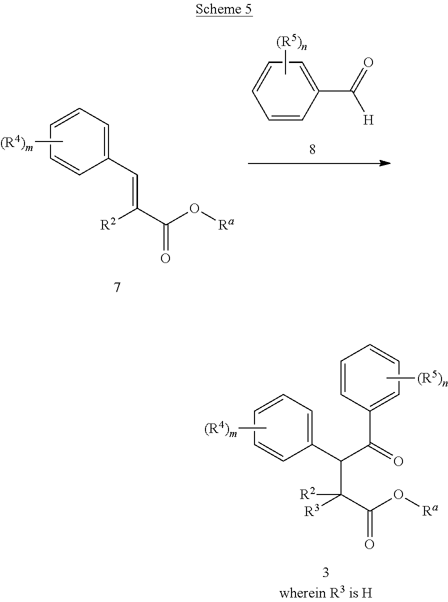

General methods useful for preparing diaryl ketones of Formula 5 are well-known in the art; see, for example, Chemical Research in Toxicology 2011, 24(11), 1853-1861; and Royal Society of Chemistry 2017, 7, 11367-11372; and references therein. Of particular note is the method illustrated in Scheme 6 below, involving reaction of a phenyl acetic acid of Formula 9 with a benzoate ester of Formula 10. Typically the reaction is run with the aid of a base and in the presence of an inert organic solvent, e.g., with sodium hydride, lithium diisopropylamide or lithium bis(trimethylsilyl)amide (LiHMDS) in a solvent such as benzene, toluene, N,N-dimethylformamide or tetrahydrofuran. The product can be isolated by adjusting the pH to about 1 to 7 and then filtering or extracting, optionally after removal of the organic solvent. Present Example 1, Step A illustrates the method of Scheme 6.

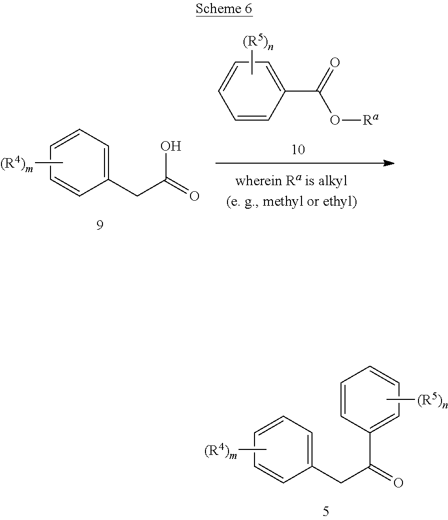

Compounds of Formula 1c (i.e. Formula 1 wherein the dotted line represents a bond, p is 0, R² is cyano and W is O) can be synthesized as outlined in Scheme 7. In the first step, compounds of Formula 12 are prepared by reaction of compounds of Formula 11 with hydrazine or hydrazine hydrate. The reaction is typically run in a solvent such as ethanol or methanol according to general procedures known in the art. Reaction of compounds of Formula 12 with cyanoacetates of Formula 13 in the presence of a base such as sodium hydride or potassium tert-butoxide and a solvent such as ethanol provides compounds of Formula 2a (i.e. Formula 2 wherein the dotted line represents a bond, p is 0, R² is cyano and W is O). Analogous to the method of Scheme 1, intermediates of Formula 2a can be alkylated to provide compounds of Formula 1c. For references see, for example, in Journal of Medicinal Chemistry 1980, 23(12), 1398-1405. Also, the method of Scheme 7 is illustrated in present Example 14, Steps C, D and E.

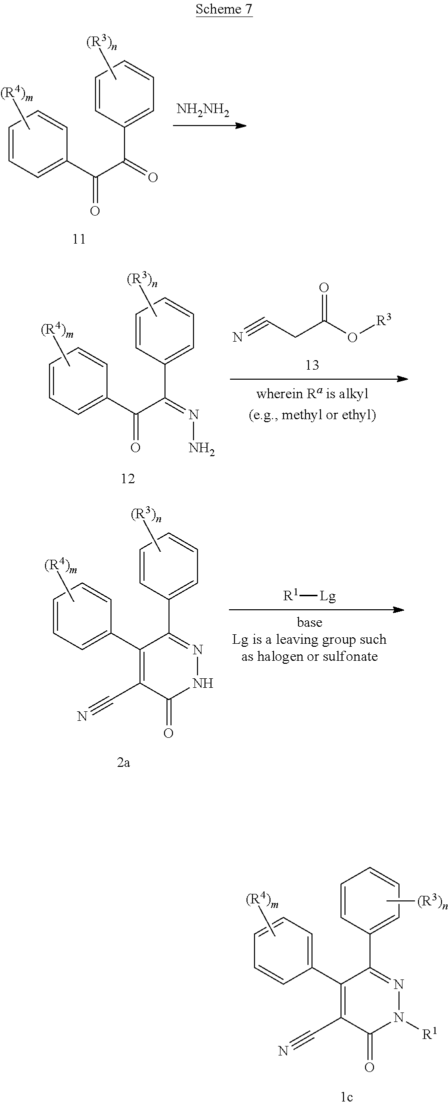

Compounds of Formula 11 are commercially available and can be prepared according to general methods known to one skilled in the art. For example, as shown in Scheme 8, catalytic oxidation of compounds of Formula 14 can be accomplished with oxygen as the oxidant in the presence of a catalyst, such as copper (II) oxide and iodine, or 1,4-diazabicyclo[2.2.2]octane (DABCO). The reaction is typically carried out in a solvent such as dimethyl sulfoxide. For reaction conditions, see, for example, Synthesis 2011, 3, 387-396; Synthesis 2013, 45(12), 1701-1707; and Journal of the American Chemical Society 2016, 138(3), 810-813. Also, present Example 14, Step B illustrates the method of Scheme 8.

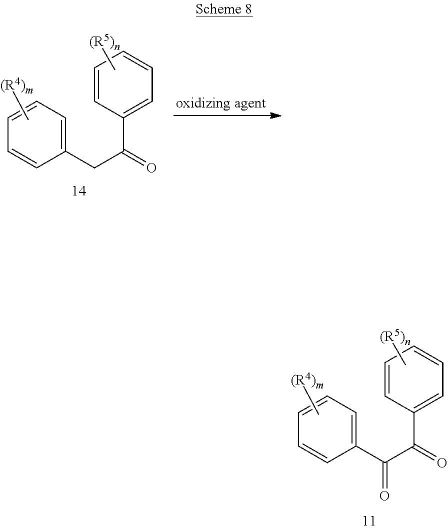

One skilled in the art will recognize that for some compounds of Formula 1 the substituents R⁴ and/or R⁵ attached to the phenyl rings may be more conveniently incorporated after forming the central pyridazinone ring with the phenyl rings attached. For example, as shown in Scheme 9, compounds of Formula 1 can undergo aromatic nitration to provide compounds of Formula 1 wherein at least one of R⁴ or R⁵ is nitro. Nitration can be accomplished according to well-known methods such as treating a compound of Formula 1 with nitric acid (or its derivatives), or a mixture of nitric acid and an acid catalyst such as sulfuric acid or acetic anhydride. One skilled in the art will recognize that certain functionalities that may be present in compounds of Formula 1 (i.e. other R⁴ and/or R⁵ substituents attached to the phenyl rings) may influence the yield of the desired products, thus requiring a suitable choice of reaction conditions. The synthetic literature includes many general methods for nitrations; see, for example, Journal of the American Chemical Society 2003,125(16), 4836-4849; Journal of Organic Chemistry 2006, 71(16) 6192-6203; and Organic Syntheses 1967, 47, 56. Also, present Examples 4 and 5 illustrate the method of Scheme 9 for adding an R⁵ nitro group using nitric acid and acetic anhydride.

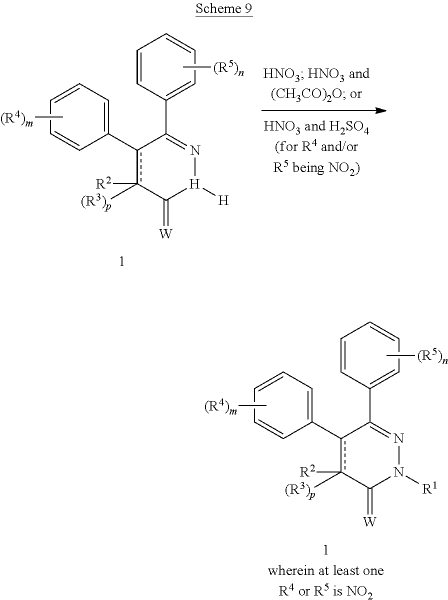

Analogous to the method of Scheme 9, compounds of Formula 1 can be treated with a halogenating agent to provide compounds of Formula 1 wherein at least one of R⁴ and/or R⁵ is halogen. A variety of halogenating agents known in the art can be used, for example, N-halosuccinimides (e.g., NBS, NCS, NIS), elemental halogen (e.g., Cl₂, Br₂, I₂) and sulfuryl chloride. Typically the reaction is carried out in a suitable solvent such as N,N-dimethylformamide, acetonitrile, dichloromethane, benzene, chlorobenzene, tetrahydrofuran. Optionally, an organic base such as triethylamine, pyridine, N,N-dimethylaniline can be added. Typical reaction temperatures range from about room temperature to 150° C. For specific reaction conditions, see present Examples 6 and 7.

Compounds of Formula 1 wherein R² is halogen or alkyl, and the like, can be prepared from corresponding compounds wherein R² is hydrogen using standard techniques known in the art. For example, as shown in Scheme 10, a compound of Formula 1d (i.e. Formula 1 wherein the dotted line represents a bond, p is 0 and R² is H) can first be treated with an organometallic agent such as an alkyl lithium base (e.g., n-butyllithium, s-butyllithium, lithium diisopropylamide or lithium tetramethylpiperidide) or a Grignard reagent (e.g., tetramethylpiperidinylmagnesium chloride) in a solvent such as toluene, ethyl ether, tetrahydrofuran or dimethoxymethane at temperatures ranging from about −78° C. to ambient temperature. Subsequent treatment with a halogenating or alkylating agent provides compounds of Formula 1e wherein R² is halogen or alkyl. For alkylation reaction conditions see, for example, Journal of Medicinal Chemistry 1980, 23(12), 1398-1405. For halogenation reaction conditions see present Examples 4, 8 and 11.

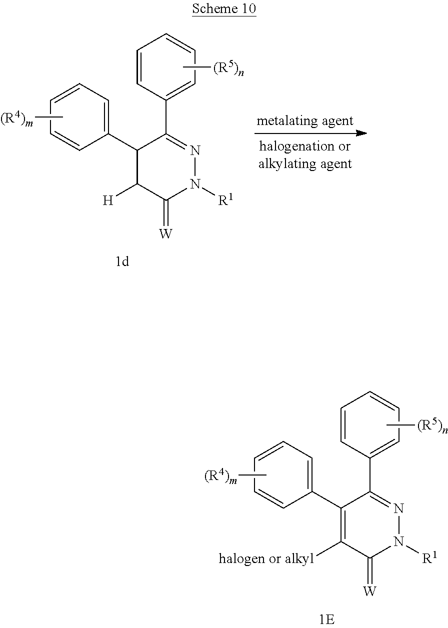

The methods of Schemes 9 and 10 are just two examples of techniques for adding substituents or modifying existing substituents in compounds of Formula 1. One skilled in the art will recognize that compounds of Formula 1 can also be subjected to numerous other electrophilic, nucleophilic, radical, organometallic, oxidation and reduction reactions to provide other functionalized compounds of Formula 1. For example, compounds of Formula 1 wherein R² is halogen can be used to prepare compounds of Formula 1 wherein R² is alkyl as illustrated in present Example 12. Compounds of Formula 1, or intermediates for their preparation, may contain aromatic nitro groups, which can be reduced to amino groups, and then converted via reactions well-known in the art (e.g., Sandmeyer reaction) to various halides. By similar known reactions, aromatic halides such as bromides or iodides prepared via the Sandmeyer reaction can react with alcohols under copper-catalyzed conditions, such as the Ullmann reaction or known modifications thereof, to provide compounds of Formula 1 that contain alkoxy substituents. Additionally, some halogen groups, such as fluorine or chlorine, can be displaced with alcohols under basic conditions to provide compounds of Formula 1 containing the corresponding alkoxy substituents. Compounds of Formula 1 or precursors thereof containing a halide, preferably bromide or iodide, are particularly useful intermediates for transition metal-catalyzed cross-coupling reactions to prepare compounds of Formula 1. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross Coupling Reactions: A Practical Guide, 2002; and references cited therein.

Compounds of Formula 1 and the intermediates described in above methods wherein W is O can be converted to the corresponding thiolates wherein W is S using a variety of standard thiating reagents such as phosphorus pentasulfide or 2,4-bis(4-methoxyphenyl)-1,3-dithia-2,4-diphosphetane-2,4-disulfide (Lawesson's reagent). Reactions of this type are well-known see, for example, Heterocycles 1995, 40, 271-278; Journal of Medicinal Chemistry 2008, 51, 8124-8134; Journal of Medicinal Chemistry 1990, 33, 2697-706; Synthesis 1989, (5), 396-3977; J. Chem. Soc., Perkin Trans. l, 1988, 1663-1668; Tetrahedron 1988 44, 3025-3036; and Journal of Organic Chemistry 1988 53(6), 1323-1326. Also, for examples relating to pyridazinones, see Journal of Heterocyclic Chemistry 1988, 25(6), 1719-23; and Phosphorus, Sulfur and Silicon and the Related Elements 2000, 156, 213-223.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after introduction of the reagents depicted in the individual schemes, additional routine synthetic steps not described in detail may be needed to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

Without further elaboration, it is believed that one skilled in the art using the preceding description can utilize the present invention to its fullest extent. The following examples are, therefore, to be construed as merely illustrative, and not limiting of the disclosure in any way whatsoever. Steps in the following examples illustrate a procedure for each step in an overall synthetic transformation, and the starting material for each step may not have necessarily been prepared by a particular preparative run whose procedure is described in other examples or steps. Ambient or room temperature is defined as about 20-25° C. Percentages are by weight except for chromatographic solvent mixtures or where otherwise indicated. Parts and percentages for chromatographic solvent mixtures are by volume unless otherwise indicated. MPLC refers to medium pressure liquid chromatography on silica gel. ¹H NMR spectra are reported in ppm downfield from tetramethylsilane; “s” means singlet, “t” means triplet, “m” means multiplet, “t” means triplet, and “br s” means broad singlet. ¹⁹F NMR spectra are reported in ppm using trichlorofluoromethane as the reference. Mass spectra are reported as the molecular weight of the highest isotopic abundance parent ion (M+1) formed by addition of H⁺ (molecular weight of 1) to the molecule, or (M−1) formed by the loss of H⁺ (molecular weight of 1) from the molecule, observed by using liquid chromatography coupled to a mass spectrometer (LCMS) using either atmospheric pressure chemical ionization (AP⁺) or electrospray ionization (ESI⁺).

### EXAMPLE 1

### Preparation of 5-(2,6-difluorophenyl)-6-(3,5-dimethoxyphenyl)-4,5-dihydro-2-methyl-3(2H)-pyridazinone (Compound 39)

### Step A: Preparation of 2-(2,6-difluorophenyl)-1-(3,5-dimethoxyphenyl)ethanone

To a solution of 2,6-difluorophenylacetic acid (12 g, 69.8 mmol) in tetrahydrofuran (200 mL) at −78° C. was added lithium bis(trimethylsilyl)amide (1.0 M in tetrahydrofuran, 209.3 mL, 209.3 mmol) dropwise. The reaction mixture was stirred at −78° C. for 1 h, and then methyl 3,5-dimethoxybenzoate (13.7 g, 69.8 mmol) in tetrahydrofuran (100 mL) was added dropwise. The reaction mixture was stirred at ambient temperature for 16 h, and then acidified with hydrochloric acid (1 N aqueous solution) to a pH of about 6. The resulting mixture was extracted with ethyl acetate (2×200 mL) and the combined organic extracts were washed with saturated aqueous sodium chloride solution (2×150 mL), dried over sodium sulfate, filtered and concentrated under reduced pressure. The resulting material was purified by MPLC (eluting with 5% ethyl acetate in petroleum ether) to yield the title compound as an off-white solid (13 g) melting at 88-92° C.

¹H NMR (DMSO-d₆) δ 7.40 (m, 1H), 7.20 (m, 2H), 7.12 (m, 2H), 6.80 (m, 1H), 4.50 (s, 2H), 3.80 (s, 6H).

LCMS: m/z: 293 [M+H]⁺

### Step B: Preparation of ethyl β-(2,6-difluorophenyl)-3,5-dimethoxy-γ-oxobenzene-butanoate

To a mixture of sodium hydride (60% in mineral oil, 1.36 g, 34.25 mmol) in dimethyl sulfoxide (76 mL) at 5° C. was added dropwise a solution of 2-(2,6-difluorophenyl)-1-(3,5-dimethoxyphenyl)ethanone (i.e. the product of Step A) (10 g, 34.25 mmol) in tetrahydrofuran (66 mL). After 1 h, ethyl bromoacetate (5.71 g, 34.25 mmol) was added dropwise to the reaction mixture. The reaction mixture was allowed to warm to room temperature, stirred for 16 h, and then poured into ice-water (200 mL). The resulting mixture was extracted with ethyl acetate (2×200 mL) and the combined organic extracts were washed with saturated aqueous sodium chloride solution (2×150 mL), dried over sodium sulfate, filtered and concentrated under reduced pressure. The resulting material was purified by MPLC (eluting with 10% ethyl acetate in petroleum ether) to provide the title compound as an off-white solid (8.0 g) melting at 100-104° C.

¹H NMR (DMSO-d₆) δ 7.36 (m, 1H), 7.10 (m, 2H), 6.90 (m, 2H), 6.70 (m, 1H), 5.20 (m, 1H), 4.07 (q, 2H), 3.70 (s, 6H), 3.15 (m, 1H), 2.75 (m, 1H), 1.13 (t, 3H).

LCMS m/z: 379 [M+H]⁺

### Step C: Preparation of β-(2,6-difluorophenyl)-3,5-dimethoxy-γ-oxobenzenebutanoic acid

To a mixture of ethyl β-(2,6-difluorophenyl)-3,5-dimethoxy-γ-oxobenzenebutanoate (i.e. the product of Step B) (8.0 g, 21.2 mmol) in tetrahydrofuran/ethanol (200 mL, 1:1) was added a solution of sodium hydroxide (1.69 g, 42.3 mmol) in water (53 mL). The reaction mixture was stirred for 16 h and then extracted with petroleum ether (2×150 mL). The combined organic extracts were further extracted with water (100 mL). The combined aqueous extracts were acidified with hydrochloric acid (1 N aqueous solution) to a pH of about 4-5. The resulting solid precipitate was collected by filtration, washed with water (2×100 mL), and dried under reduced pressure to provide the title compound as a white solid (5.5 g).

¹H NMR (DMSO-d₆) δ 12.4 (br s, 1H), 7.36 (m, 1H), 7.10 (m, 2H), 6.90 (m, 2H), 6.70 (m, 1H), 5.15 (m, 1H), 3.73 (s, 6H), 3.15 (m, 1H), 2.64 (m, 1H).

LCMS m/z: 351 [M+H]⁺

### Step D: Preparation of 5-(2,6-difluorophenyl)-6-(3,5-dimethoxyphenyl)-4,5-dihydro-2-methyl-3(2H)-pyridazinone

To a mixture of β-(2,6-difluorophenyl)-3,5-dimethoxy-γ-oxobenzenebutanoic acid (i.e. the product of Step C) (2.0 g, 5.7 mmol) in ethanol (20 mL) was added methyl hydrazine (85% aqueous solution, 0.611 g, 11.4 mmol). The reaction mixture was heated in a sealed tube at 100° C. in a microwave reactor for 3 h, and then poured into ice-water (150 mL). The resulting solid precipitate was collected by filtration, washed with water (2×20 mL) and dried under reduced pressure. The solid was then triturated with diethyl ether (2×10 mL), filtered and dried to provide the title compound, a compound of the present invention, as an off-white solid (1.5 g) melting at 139-142° C.

¹H NMR (DMSO-d₆) δ 7.35 (m, 1H), 7.10 (m, 2H), 6.73 (m, 2H), 6.48 (m, 1H), 5.03 (m, 1H), 3.70 (s, 6H), 3.40 (s, 3H), 3.15 (m, 1H), 2.54 (m, 1H).

LCMS m/z: 361 [M+H]⁺

### EXAMPLE 2

### Preparation of 5-(2,6-difluorophenyl)-6-(3,5-dimethoxyphenyl)-2-methyl-3(2H)-pyridazinone (Compound 38)

To a mixture of 5-(2,6-difluorophenyl)-6-(3,5-dimethoxyphenyl)-4,5-dihydro-2-methyl-3(2H)-pyridazinone (i.e. the product of Example 1) (500 mg, 1.39 mmol) in chlorobenzene (15 mL) was added manganese dioxide (1.79 g, 20.8 mmol). The reaction mixture was heated in a sealed tube at 100° C. for 16 h, cooled to room temperature, and filtered through Celite® diatomaceous earth filter aid, rinsing with ethyl acetate (2×50 mL). The filtrate was concentrated under reduced pressure and the resulting material was purified by MPLC (eluting with 20% ethyl acetate in petroleum ether) to provide the title compound, a compound of the present invention, as an off-white solid (0.30 g) melting at 132-136° C.

¹H NMR (DMSO-d6) δ 7.34 (m, 1H), 7.00 (s, 1H), 6.90 (m, 2H), 6.40-6.35 (m, 3H), 3.92 (s, 3H), 3.64 (s, 6H).

LCMS: m/z: 359 [M+H]⁺

### EXAMPLE 3

### Preparation of 4-chloro-5-(2,6-difluorophenyl)-6-(3,5-dimethoxyphenyl)-2-methyl-3(2H)-pyridazinone (Compound 36)

To a mixture of 5-(2,6-difluorophenyl)-6-(3,5-dimethoxyphenyl)-2-methyl-3(2H)-pyridazinone (i.e. the product of Example 2) (1.5 g, 4.19 mmol) in tetrahydrofuran (12 mL) at −20° C. was added 2,2,6,6-tetramethylpiperidinylmagnesium chloride lithium chloride complex (1 M solution in tetrahydrofuran, 5.0 mL, 5.0 mmol). The reaction mixture was stirred for 1 h at −20° C., and then benzenesulfonyl chloride (0.76 g, 4.3 mmol) was added. The reaction mixture was allowed to warm to 0° C. and stirred for 2 h, and then poured into ice-water (100 mL). The resulting mixture was extracted with ethyl acetate (2×100 mL) and the combined organic extracts were washed with saturated aqueous sodium chloride solution (2×150 mL), dried over sodium sulfate, filtered and concentrated under reduced pressure. The resulting material was purified by MPLC (eluting with 20% ethyl acetate in petroleum ether) to provide the title compound, a compound of the present invention, as a solid (0.40 g) melting at 150-154° C.

¹H NMR (DMSO-d6) δ 7.35 (m, 1H), 6.90 (m, 2H), 6.40-6.35 (m, 3H), 3.97 (s, 3H), 3.65 (s, 6H).

LCMS: m/z: 393 [M+H]⁺

### EXAMPLE 4

### Preparation of 4-chloro-5-(2,6-difluorophenyl)-6-(3,5-dimethoxy-2-nitrophenyl)-2-methyl-3(2H)-pyridazinone (Compound 37)

To a mixture of 4-chloro-5-(2,6-difluorophenyl)-6-(3,5-dimethoxyphenyl)-2-methyl-3(2H)-pyridazinone (i.e. the product of Example 3) (200 mg, 0.51 mmol) in acetic anhydride (0.6 mL) at −40° C. was added concentrated nitric acid (0.06 mL). After 10 minutes at −40° C., the reaction mixture was poured into ice-water (50 mL). The resulting solid precipitate was collected by filtration, washed with water (2×10 mL) and dried under reduced pressure. The solid was then triturated with n-pentane (20 mL), filtered and dried to provide the title compound, a compound of the present invention, as an off-white solid (104 mg) melting at 223-227° C.

¹H NMR (DMSO-d6): δ 7.35 (m, 1H), 7.10 (m, 2H), 6.73 (m, 2H), 6.48 (m, 1H), 5.03 (m, 1H), 3.70 (s, 6H), 3.40 (s, 3H), 3.15 (m, 1H), 2.54 (m, 1H).

LCMS: m/z: 438 [M+H]⁺

### EXAMPLE 5

### Preparation of 5-(2,6-difluorophenyl)-6-(3,5-dimethoxy-2-nitrophenyl)-2-methyl-3(2H)-pyridazinone (Compound 13)

To a mixture of 5-(2,6-difluorophenyl)-6-(3,5-dimethoxyphenyl)-2-methyl-3(2H)-pyridazinone (i.e. the product of Example 2) (500 mg, 1.40 mmol) in acetic anhydride (1.5 mL) at −40° C. was added concentrated nitric acid (0.15 mL). After 10 minutes at −40° C., the reaction mixture was poured into ice-water (50 mL). The resulting solid precipitate was collected by filtration, rinsed with water (2×10 mL) and dried under reduced pressure. The solid was purified by MPLC (eluting with 25% ethyl acetate in petroleum ether) to provide the title compound, a compound of the present invention, as an off-white solid (280 mg) melting at 174-178° C.

¹H NMR (CDCl₃) δ 7.35 (m, 1H), 7.00 (s, 1H), 6.90 (m, 2H), 6.48 (m, 1H), 6.23 (m, 1H), 3.85 (s, 6H), 3.67 (s, 3H).

LCMS: m/z: 404 [M+H]⁺

### EXAMPLE 6

### Preparation of 4-chloro-6-(2-chloro-3,5-dimethoxyphenyl)-5-(2,6-difluorophenyl)-2-methyl-3(2H)-pyridazinone (Compound 35)

To a mixture of 4-chloro-5-(2,6-difluorophenyl)-6-(3,5-dimethoxyphenyl)-2-methyl-3(2H)-pyridazinone (i.e. the product of Example 3) (600 mg, 1.53 mmol) in acetonitrile (10 mL) was added N-chlorosuccinimide (224 mg, 1.68 mmol). The reaction mixture was heated at 80° C. for 16 h, cooled and then poured into ice-water (100 mL). The resulting mixture was extracted with ethyl acetate (2×150 mL), and the combined organic extracts were washed with saturated aqueous sodium chloride solution (2×50 mL), dried over sodium sulfate, filtered and concentrated under reduced pressure. The resulting material was purified by MPLC (eluting with 30% ethyl acetate in petroleum ether) to provide the title compound, a compound of the present invention, as an off-white solid (290 mg) melting at 192-196° C.

¹H NMR (CDCl₃) δ 7.30 (m, 1H), 6.88-6.80 (br s, 2H), 6.46-6.43 (m, 2H), 3.96 (s, 3H), 3.79 (s, 3H), 3.74 (s, 3H).

LCMS: m/z: 427 [M+H]⁺

### EXAMPLE 7

### Preparation of 6-(2-chloro-3,5-dimethoxyphenyl)-5-(2,6-difluorophenyl)-2-methyl-3(2H)-pyridazinone (Compound 12) and 6-(4-chloro-3,5-dimethoxyphenyl)-5-(2,6-difluorophenyl)-2-methyl-3(2H)-pyridazinone (Compound 14)

To a mixture of 5-(2,6-difluorophenyl)-6-(3,5-dimethoxyphenyl)-2-methyl-3(2H)-pyridazinone (i.e. the product of Example 2) (600 mg, 1.68 mmol) in acetonitrile (10 mL) was added N-chlorosuccinimide (226 mg, 1.68 mmol). The reaction mixture was heated at 80° C. for 16 h, cooled to room temperature and poured into ice-water (100 mL). The resulting mixture was extracted with ethyl acetate (2×150 mL), and the combined organic extracts were washed with saturated aqueous sodium chloride solution (2×50 mL), dried over sodium sulfate, filtered and concentrated under reduced pressure. The resulting material was purified by supercritical fluid chromatography to provide 6-(2-chloro-3,5-dimethoxyphenyl)-5-(2,6-difluorophenyl)-2-methyl-3(2H)-pyridazinone, a compound of the present invention, as an off-white solid (280 mg) melting at 172-176° C.

¹H NMR (CDCl₃) δ 7.25 (m, 1H), 7.00 (s, 1H), 6.80 (br s, 2H), 6.51 (m, 1H), 6.45 (m, 1H), 3.90 (s, 3H), 3.78 (s, 3H), 3.76 (s, 3H).

LCMS: m/z: 393 [M+H]⁺

Also isolated was 6-(4-chloro-3,5-dimethoxyphenyl)-5-(2,6-difluorophenyl)-2-methyl-3(2H)-pyridazinone, a compound of the present invention, as a white solid (40 mg) melting at 221-225° C.

1H NMR (CDCl₃) δ 7.35 (m, 1H), 7.02 (s, 1H), 6.90 (m, 2H), 6.46 (s, 2H), 3.93 (s, 3H), 3.70 (s, 6H).

LCMS: m/z: 393 [M+H]⁺

### EXAMPLE 8

### Preparation of 4-bromo-5-(2,6-difluorophenyl)-6-(3,5-dimethoxyphenyl)-2-methyl-3(2H)-pyridazinone (Compound 34)

To a mixture of 5-(2,6-difluorophenyl)-6-(3,5-dimethoxyphenyl)-2-methyl-3(2H)-pyridazinone (i.e. the product of Example 2) (300 mg, 0.84 mmol) in tetrahydrofuran (2.5 mL) at −20° C. was added 2,2,6,6-tetramethylpiperidinylmagnesium chloride lithium chloride complex (1 M in tetrahydrofuran, 1.25 mL, 1.25 mmol). The reaction mixture was stirred at −20° C. for 1 h, and then 1,3-dibromo-5,5-dimethyl-2,4-imidazolidinedione (251 mg, 0.88 mmol) in tetrahydrofuran (1 mL) was added dropwise. After stirring for an additional 2 h at 0° C., the reaction mixture was poured into ice-water (10 mL) and extracted with ethyl acetate (2×50 mL). The combined organic extracts were washed with saturated aqueous sodium chloride solution (2×20 mL), dried over sodium sulfate, filtered and concentrated under reduced pressure. The resulting material was purified by MPLC (eluting with 20% ethyl acetate in petroleum ether) to provide the title compound, a compound of the present invention, as an off-white solid (70 mg) melting at 153-157° C.

¹H NMR (CDCl₃) δ 7.35 (m, 1H), 6.90 (m, 2H), 6.35-6.30 (m, 3H), 3.97 (s, 3H), 3.66 (s, 6H).

LCMS: m/z: 437 [M+H]⁺

### EXAMPLE 9

### Preparation of 5,6-bis(2,6-difluorophenyl)-2-methyl-3(2H)-pyridazinone (Compound 5)

### Step A: Preparation of 5,6-dichloro-2-methyl-3(2H)-pyridazinone

To a mixture of 5,6-dichloro-3(2H)-pyridazinone (5.3 g, 32.1 mmol) in N,N-dimethylformamide (65 mL) was added cesium carbonate (12.5 g, 37.8 mmol) and iodomethane (2.6 mL, 41.5 mmol). The reaction mixture was stirred for 16 h, and then partitioned between ethyl acetate (300 mL) and water (150 mL). The layers were separated, and the organic layer was washed with water (5×100 mL), dried over sodium sulfate, filtered and concentrated under reduced pressure to provide the title compound as a white solid (4.6 g).

¹H NMR (CDCl₃) δ 7.10 (s, 1H), 3.80 (s, 3H).

LCMS: m/z: 179 [M+H]⁺

### Step B: Preparation of 5,6-bis(2,6-difluorophenyl)-2-methyl-3(2H)-pyridazinone (Compound 5)

To a mixture of 2-bromo-1,3-difluorobenzene (2.5 g, 12.9 mmol) in tetrahydrofuran (17 mL) at −78° C. was added n-butyllithium (1.6 M in hexanes, 9.0 mL, 14.2 mmol) dropwise. The reaction mixture was stirred at −78° C. for 1 h, then zinc chloride (1.9 M in 2-methyltetrahydrofuran, 8.2 mL, 15.5 mmol) was added dropwise and the mixture was allowed to gradually warm to room temperature. After 1 h, 5,6-dichloro-2-methyl-3(2H)-pyridazinone (i.e. the product of Step A) (1.0 g, 5.6 mmol), dicyclohexyl[2′,4′,6′-tris(1-methylethyl)[1,1′-biphenyl]-2-yl]-phosphine (53.0 mg, 0.11 mmol) and (2-dicyclohexylphosphino-2′,4′,6′-triisopropyl-1,1′-biphenyl)[2-(2′-amino-1,1′-biphenyl)]palladium (II) methanesulfonate (93.0 mg, 0.11 mmol) were added to the reaction mixture. After 16 h, the reaction mixture was diluted with water (20 mL) and ethyl acetate (50 mL), the organic layer was separated, dried over sodium sulfate, filtered and concentrated under reduced pressure. The resulting material was purified by silica gel column chromatography (eluting with a gradient of 0 to 100% ethyl acetate in hexanes) to provide the title compound, a compound of the present invention, as a white solid (949 mg).

¹H NMR (CDCl₃) δ 7.30 (m, 2H), 7.10 (s, 1H), 6.80 (m, 4H), 3.90 (s, 3H).

### EXAMPLE 10

### Preparation of 6-(2,6-difluorophenyl)-5-(3,5-dimethoxyphenyl)-2-ethyl-3(2H)-pyridazinone (Compound 4)

### Step A: Preparation of 5,6-dichloro-2-ethyl-3(2H)-pyridazinone

To a mixture of 5,6-dichloro-3(2H)-pyridazinone (15 g, 91 mmol) in N,N-dimethylformamide (182 mL) was added cesium carbonate (36 g, 109 mmol) and iodoethane (9.5 mL, 118 mmol). After 16 h, the reaction mixture was partitioned between ethyl acetate (500 mL) and water (200 mL), the layers were separated and the organic layer was washed with water (5×100 mL), dried over sodium sulfate, filtered and concentrated under reduced pressure to provide the title compound as a white solid (14.9 g).

¹H NMR (CDCl₃) δ 7.07 (s, 1H), 4.17 (q, 2H), 1.38 (t, 3H).

### Step B: Preparation of 6-chloro-5-(3,5-dimethoxyphenyl)-2-ethyl-3 (2H)-pyridazinone

A mixture of 5,6-dichloro-2-ethyl-3(2H)-pyridazinone (i.e. the product of Step A) (1.0 g, 5.2 mmol), 3,5-dimethoxyphenylboronic acid (1.0 g, 5.7 mmol), tetrakis(triphenylphosphine)-palladium(0) (0.6 g, 0.5 mmol) and sodium carbonate (1.1 g, 10.4 mmol) in a solution of toluene (20 mL), ethanol (5 mL) and water (5 mL) was stirred under a stream of nitrogen gas for 1 h, and then heated at 90° C. for 16 h. After cooling to room temperature, the reaction mixture was diluted with ethyl acetate (150 mL) and water (50 mL). The organic layer was separated, washed with saturated aqueous sodium chloride solution (50 mL), dried over sodium sulfate, filtered and concentrated under reduced pressure. The resulting material was purified by silica gel column chromatography (eluting with a gradient of 0 to 100% ethyl acetate in hexanes) to provide the title compound as a white solid (576 mg).

¹H NMR (CDCl₃) δ 6.90 (s, 1H), 6.54 (s, 3H), 4.22 (q, 2H), 3.81 (s, 6H), 1.43 (t, 3H).

LCMS m/z: 295 [M+H]⁺

### Step C: Preparation of 6-(2,6-difluorophenyl)-5-(3,5-dimethoxyphenyl)-2-ethyl-3(2H)-pyridazinone

To a mixture of 2-bromo-1,3-difluorobenzene (448 mg, 2.32 mmol) in tetrahydrofuran (3 mL) at −78° C. was added n-butyllithium (2.5 M in hexanes, 1.0 mL, 2.56 mmol) dropwise. The reaction mixture was stirred at −78° C. for 1 h, then zinc chloride (1.9 M in 2-methyltetrahydrofuran, 1.5 mL, 2.8 mmol) was added dropwise and the mixture was allowed to gradually warm to room temperature. After 1 h, 6-chloro-5-(3,5-dimethoxyphenyl)-2-ethyl-3(2H)-pyridazinone (i.e. the product of Step B) (0.3 g, 1.0 mmol), dicyclohexyl[2′,4′,6′-tris(1-methylethyl)[1,1′-biphenyl]-2-yl]-phosphine (24.1 mg, 0.05 mmol) and (2-dicyclohexyl-phosphino-2′,4′,6′-triisopropyl-1,1′-biphenyl) [2-(2′-amino-1,1′-biphenyl)]palladium (II) methane-sulfonate (43.2 mg, 0.05 mmol) were added to the reaction mixture. After 16 h, the reaction mixture was diluted with water (20 mL) and ethyl acetate (50 mL), the organic layer was separated, dried over sodium sulfate, filtered and concentrated under reduced pressure. The resulting material was purified by silica gel column chromatography (eluting with a gradient of 0 to 100% ethyl acetate in hexanes) to provide the title compound, a compound of the present invention, as a white solid (313 mg).

¹H NMR (CDCl₃) δ 7.32 (m, 1H), 6.97 (s, 1H), 6.87 (m, 2H), 6.37 (m, 1H), 6.26 (m, 2H), 4.33 (q, 2H), 3.62 (s, 6H), 1.45 (t, 3H).

¹⁹F NMR (CDCl₃) δ-112.08.

LCMS m/z: 373 [M+H]⁺

### EXAMPLE 11

### Preparation of 4-chloro-6-(2,6-difluorophenyl)-5-(3,5-dimethoxyphenyl)-2-ethyl-3(2H)-pyridazinone (Compound 10)

To a mixture of 6-(2,6-difluorophenyl)-5-(3,5-dimethoxyphenyl)-2-ethyl-3(2H)-pyridazinone (i.e. the product of Example 10) (154.0 mg, 0.4 mmol) in tetrahydrofuran (0.2 mL) at −20° C. was added 2,2,6,6-tetramethylpiperidinylmagnesium chloride lithium chloride complex (1.0 M solution in tetrahydrofuran/toluene, 0.6 mL, 0.6 mmol). The reaction mixture was stirred at −20° C. for 30 minutes, then benzenesulfonyl chloride (63 μL, 0.5 mmol) was added and the mixture was allowed to gradually warm to room temperature. After 4 h, the reaction mixture was cooled to 0° C. and more 2,2,6,6-tetramethylpiperidinylmagnesium chloride lithium chloride complex (1.0 M solution in tetrahydrofuran/toluene, 0.6 mL, 0.6 mmol) was added. After stirring for 1 h at 0° C., more benzenesulfonyl chloride (63 μL, 0.5 mmol) was added to the reaction mixture, and the mixture was allowed to gradually warm to room temperature. After 16 h, water (15 mL) and ethyl acetate (50 mL) were added to the reaction mixture, the organic layer was separated, dried over sodium sulfate, filtered and concentrated under reduced pressure. The resulting material was purified by silica gel column chromatography (eluting with a gradient of 0 to 100% ethyl acetate in hexanes) to provide the title compound, a compound of the present invention, as a white solid (26.6 mg).

¹H NMR (CDCl₃) δ 7.28 (m, 1H), 6.83 (m, 2H), 6.36 (m, 1H), 6.29 (m, 2H), 4.39 (q, 2H), 3.69 (s, 6H), 1.49 (t, 3H).

¹⁹F NMR (CDCl₃) δ-111.07.

LCMS m/z: 408 [M+H]⁺

### EXAMPLE 12

### Preparation of 5-(2,6-difluorophenyl)-6-(3,5-dimethoxyphenyl)-2,4-dimethyl-3 (2H)-pyridazinone (Compound 21)

To a mixture of 4-bromo-5-(2,6-difluorophenyl)-6-(3,5-dimethoxyphenyl)-2-methyl-3(2H)-pyridazinone (i.e. the product of Example 8) (90 mg, 0.20 mmol) in 1,4-dioxane (1 mL) was added water (2 drops), dichloro[1,1′-bis(diphenylphosphino)ferrocene]palladium (II) dichloromethane complex (17 mg, 0.020 mmol), cesium carbonate (130 mg, 0.40 mmol) and 2,4,6-trimethylboroxine (147 μL, 1.05 mmol). The reaction mixture was heated at 100° C. for 4 h, cooled to room temperature and filtered through Celite® diatomaceous earth filter aid, rinsing with ethyl acetate. The filtrate was concentrated under reduced pressure and the resulting material was purified by silica gel column chromatography (eluting with a gradient of 5 to 100% ethyl acetate in hexanes) to provide the title compound, a compound of the present invention, as a white solid (73 mg).

¹H NMR (DMSO-d6) δ 7.50 (m, 1H), 7.20 (m, 2H), 6.42 (s, 1H), 6.25 (s, 2H), 3.79 (s, 3H), 3.60 (s, 6H), 1.95 (s, 3H).

LCMS m/z: 373 [M+H]⁺

### EXAMPLE 13

### Preparation of 5-(2,6-difluorophenyl)-6-(3,5-dimethoxyphenyl)-4,5-dihydro-2-ethyl-3(2H)-pyridazinone (Compound 33)

To a mixture of β-(2,6-difluorophenyl)-3,5-dimethoxy-γ-oxobenzenebutanoic acid (i.e. the product of Example 1, Step C) (5.0 g, 14.3 mmol) in pyridine (35 mL) was added ethylhydrazine hydrochloride (2.74 g, 28.6 mmol). The reaction mixture was heated at 100° C. for 3 days, poured into ice-water (200 mL) and extracted with ethyl acetate (2×200 mL). The combined organic extracts were washed with saturated aqueous sodium chloride solution (2×150 mL), dried over sodium sulfate, filtered and concentrated under reduced pressure. The resulting material was purified by silica gel column chromatography (eluting with 30% ethyl acetate in hexanes) to provide the title compound, a compound of the present invention, as an off-white solid (1.6 g) melting at 119-122° C.

¹H NMR (CDCl₃) δ 7.18 (m, 1H), 6.84 (m, 2H), 6.77 (s, 2H), 6.40 (m, 1H), 4.82 (m, 1H), 4.08 (m, 1H), 3.88 (m, 1H), 3.74 (s, 6H), 2.98 (m, 1H), 2.74 (m, 1H), 1.33 (t, 3H).

LCMS: m/z: 375 [M+H]⁺

### EXAMPLE 14

### Preparation of 5,6-bis(2-chloro-4-fluorophenyl)-2,3-dihydro-2-methyl-3-oxo-4-pyridazine-carbonitrile (Compound 6)

### Step A: Preparation of 1,2-bis(2-chloro-4-fluorophenyl)ethanone

To a solution of 2-chloro-4-fluorobenzeneacetic acid (5.0 g, 26.5 mmol) in tetrahydrofuran (100 mL) at −78° C. was added lithium bis(trimethylsilyl)amide (1.0 M in tetrahydrofuran, 80 mL, 80 mmol) dropwise. The reaction mixture was stirred at −78° C. for 1 h, then methyl 2-chloro-4-fluorobenzoate (5.0 g, 26.5 mmol) in tetrahydrofuran (50 mL) was added dropwise and the mixture was allowed to warm to ambient temperature. The reaction mixture was stirred at room temperature for 16 h, and then acidified with hydrochloric acid (1 N aqueous solution) to a pH of about 6. The resulting mixture was extracted with ethyl acetate (2×200 mL) and the combined organic extracts were washed with saturated aqueous sodium chloride solution (2×150 mL), dried over sodium sulfate, filtered and concentrated under reduced pressure. The resulting material was purified by MPLC (eluting with 5% ethyl acetate in petroleum ether) to provide the title compound as an oil (5 g).

¹H NMR (CDCl₃) δ 7.80 (m, 1H), 7.25 (m, 1H), 7.20-7.10 (m, 2H), 7.10-7.00 (m, 1H), 7.00 (m, 1H), 4.38 (s, 2H).

### Step B: Preparation of 1,2-bis(2-chloro-4-fluorophenyl)-1,2-ethanedione

To a solution of 1,2-bis(2-chloro-4-fluorophenyl) ethanone (i.e. the product of Step A) (5.00 g, 16.7 mmol) in dimethyl sulfoxide (80 mL) at 5° C. was added copper (II) oxide (1.32 g, 16.7 mmol) and iodine (4.62 g, 36.66 mmol). The reaction mixture was heated at 100° C. for 6 h under an oxygen atmosphere, and then cooled to room temperature and treated with saturated aqueous sodium thiosulfate solution (100 mL). The resulting mixture was extracted with ethyl acetate (2×200 mL) and the combined organic extracts were washed with saturated aqueous sodium chloride solution (2×50 mL), dried over sodium sulfate, filtered and concentrated under reduced pressure. The resulting material was purified by MPLC (eluting with 20% ethyl acetate in petroleum ether) to provide the title compound as a yellow solid (4.0 g).

### Step C: Preparation of 1,2-bis(2-chloro-4-fluorophenyl)-1,2-ethanedione 1-hydrazone

To a solution of 1,2-bis(2-chloro-4-fluorophenyl)-1,2-ethanedione (i.e. the product of Step B) (300 mg, 0.955 mmol) in methanol (5 mL) was added hydrazine hydrate (0.071 mL, 1.4 mmol). The reaction mixture was heated at reflux for 15 minutes and then cooled to ambient temperature. The resulting solid precipitate was collected by filtration and dried under reduced pressure to provide the title compound as a white solid (0.25 g).

### Step D: Preparation of 5,6-bis(2-chloro-4-fluorophenyl)-2,3-dihydro-3-oxo-4-pyridazine-carbonitrile

To a mixture of sodium metal (0.22 g, 9.4 mmol) in ethanol (30 mL), cooled an ice bath, was added ethyl cyanoacetate (1.0 mL, 9.4 mmol). The reaction mixture was stirred for 30 minutes and allowed to warm to room temperature, and then 1,2-bis(2-chloro-4-fluorophenyl)-1,2-ethanedione 1-hydrazone (i.e. the product of Step C) (2.8 g, 8.5 mmol) was added. The reaction mixture was heated at reflux for 6 h, cooled to room temperature, and then acidified with hydrochloric acid (1 N aqueous solution) to a pH of about 4-5. The resulting mixture was extracted with ethyl acetate (2×100 mL) and the combined organic extracts were washed with saturated aqueous sodium chloride solution (2×50 mL), dried over sodium sulfate, filtered and concentrated under reduced pressure. The resulting material was purified by MPLC (eluting with 30% ethyl acetate in petroleum ether) to provide the title compound as an off-white solid (0.91 g).

### Step E: Preparation of 5,6-bis(2-chloro-4-fluorophenyl)-2,3-dihydro-2-methyl-3-oxo pyridazinecarbonitrile

To a mixture of 5,6-bis(2-chloro-4-fluorophenyl)-2,3-dihydro-3-oxo-4-pyridazine-carbonitrile (i.e. the product of Step D) (750 mg, 1.99 mmol) in N,N-dimethylformamide (5 mL) was added potassium carbonate (549 mg, 3.98 mmol) and iodomethane (0.185 mL, 2.98 mmol). After 2 h, the reaction mixture was poured into ice-water (50 mL) and extracted with ethyl acetate (2×100 mL). The combined organic extracts were washed with saturated aqueous sodium chloride solution (2×50 mL), dried over sodium sulfate, filtered and concentrated under reduced pressure. The resulting material was purified by MPLC (eluting with 20% ethyl acetate in petroleum ether) to provide the title compound, a compound of the present invention, as an off-white solid (238 mg) melting at 137-140° C.

¹H NMR (DMSO-d₆) δ 7.60 (m, 1H), 7.55 (m, 1H), 7.49-7.42 (m, 2H), 7.34 (m, 1H), 7.25 (m, 1H), 3.87 (s, 3H), 3.32 (s, 6H).

LCMS: m/z: 392 [M+H]⁺

### EXAMPLE 15

### Preparation of 5-(2-chloro-4-fluorophenyl)-6-(2-chloro-5-methylphenyl)-2,4-dimethyl-3(2H)-pyridazinone (Compound 136)

### Step A: Preparation of 6-chloro-5-(2-chloro-4-fluorophenyl)-2-methyl-3(2H)-pyridazinone

A mixture of 5,6-dichloro-2-methyl-3(2H)-pyridazinone (2.0 g, 11.2 mmol), 2-chloro-4-fluorophenylboronic acid (2.1 g, 11.7 mmol), sodium carbonate (4.9 mL, 2.0 M solution in water) and bis(triphenylphosphine)palladium (II) dichloride (1.57 g, 2.24 mmol) in dioxane (78.1 mL) was heated at 100° C. for 16 h. After cooling to room temperature, the mixture was diluted with water and ethyl acetate. The layers were separated, and the aqueous layer was extracted with ethyl acetate. The combined organics were dried over sodium sulfate, filtered and concentrated under reduced pressure. The resulting material was purified by silica gel column chromatography (eluting with a gradient of 0 to 40% ethyl acetate in hexanes) to provide the title compound as a solid (1.68 g).

¹H NMR (CDCl₃) δ 7.28-7.21 (m, 2H), 7.13-7.09 (m, 1H), 6.87 (s, 1H), 3.82 (s, 3H).

### Step B: Preparation of 6-chloro-5-(2-chloro-4-fluorophenyl)-2,4-dimethyl-3(2H)-pyridazinone

To a mixture of 6-chloro-5-(2-chloro-4-fluorophenyl)-2-methyl-3(2H)-pyridazinone (i.e. the product of Step A) (5.0 g, 18.3 mmol) in tetrahydrofuran (183 mL) at −20° C. was added methylmagnesium bromide (21.5 mL, 3.4 M solution in tetrahydrofuran). The reaction mixture was stirred for 10 minutes, and then bromine (3.8 mL, 73.2 mmol) was added. The reaction mixture was allowed to gradually warm to room temperature and more tetrahydrofuran (30 mL) was added to facilitate stirring. After 3 h, the reaction mixture was poured into sodium thiosulfate solution. The resulting mixture was extracted with ethyl acetate and the combined organic extracts were washed with water, dried, filtered and concentrated under reduced pressure. The resulting material was purified by silica gel column chromatography (eluting with a gradient of 0 to 40% ethyl acetate in hexanes) to provide the title compound as a solid (4.57 g).

¹H NMR (CDCl₃) δ 7.30-7.28 (m, 1H), 7.16-7.11 (m, 2H), 3.83 (s, 3H), 1.99 (s, 3H).

### Step C: Preparation of 5-(2-chloro-4-fluorophenyl)-6-(2-chloro-5-methylphenyl)-2,4-dimethyl-3(2H)-pyridazinone

A mixture of 6-chloro-5-(2-chloro-4-fluorophenyl)-2,4-dimethyl-3(2H)-pyridazinone (i.e. the product of Step B) (0.3 g, 1.05 mmol), 2-chloro-5-methylphenylboronic acid (0.19 g, 1.1 mmol), sodium carbonate (0.46 mL, 2.0 M solution in water) and bis(triphenylphosphine)-palladium (II) dichloride (0.15 g, 0.21 mmol) in dioxane (7.3 mL) was heated at 100° C. for 16 h. The reaction mixture was cooled to room temperature, and then partitioned between ethyl acetate and water. The layers were separated, and the aqueous layer was extracted with ethyl acetate. The combined organics were dried over sodium sulfate, filtered and concentrated under reduced pressure. The resulting material was purified by silica gel column chromatography (eluting with a gradient of 0 to 100% ethyl acetate in hexanes) to provide the title compound, a compound of the present invention, as an oil (75 mg).

¹H NMR (CDCl₃) δ 7.12 (m, 2H), 7.07-7.04 (m, 2H), 7.00-6.98 (m, 1H), 6.89-6.86 (m, 1H), 3.91 (s, 3H), 2.23 (s, 3H), 2.01 (s, 3H).

### EXAMPLE 16

### Preparation of 4-chloro-5-(2-chloro-4-fluorophenyl)-2-methyl-6-phenyl-3(2H)-pyridazinone (Compound 141)

### Step A: Preparation of 4-chloro-5-iodo-2-methyl-3(2H)-pyridazinone

To a mixture of 4,5-dichloro-2-methyl-3(2H)-pyridazinone (7.32 g, 40.9 mmol) in N,N-dimethylformamide (68 mL) was added sodium iodide (24.5 g, 163 mmol). The reaction mixture was heated at 150° C. for 16 h, after which time more sodium iodide (6.13 g, 40.9 mmol) was added to the reaction mixture. After stirring at 150° C. for an additional 6 h, more sodium iodide (6.13 g, 40.9 mmol) was added to the reaction mixture and stirring was continue at 150° C. for an additional 20 h. After cooling to room temperature, the reaction mixture was partitioned between ethyl acetate and water. The layers were separated, and the aqueous layer was extracted with ethyl acetate. The combined organics were dried over sodium sulfate, filtered and concentrated under reduced pressure. The resulting solid (7.8 g) was used in the next step without further purification.

¹H NMR (CDCl₃) δ 7.77 (s, 1H), 3.82 (s, 3H).

### Step B: Preparation of 4-chloro-5-(2-chloro-4-fluorophenyl)-2-methyl-3(2H)-pyridazinone

A mixture of 4-chloro-5-iodo-2-methyl-3(2H)-pyridazinone (i.e. the product of Step A) (5.0 g, 18.5 mmol), 2-chloro-4-fluorophenylboronic acid (3.55 g, 20.3 mmol), bis(triphenyl-phosphine)palladium (II) dichloride (2.6 g, 3.7 mmol) and sodium carbonate (8.14 mL, 2 M solution in water) in dioxane (129 mL, 0.14 M) was heated at 100° C. for 16 h. After cooling to room temperature, the reaction mixture was partitioned between ethyl acetate and water. The layers were separated, and the aqueous layer was extracted with ethyl acetate. The combined organics were dried over sodium sulfate, filtered and concentrated under reduced pressure. The resulting material was purified by silica gel column chromatography (eluting with a gradient of 0 to 60% ethyl acetate in hexanes) to provide the title compound as a solid (3.5 g).

¹H NMR (CDCl₃) δ 7.64 (s, 1H), 7.31-7.28 (2H, m), 7.15-7.12 (1H, m), 3.9 (s, 3H).

### Step C: Preparation of 4-chloro-5-(2-chloro-4-fluorophenyl)-2-methyl-6-phenyl-3(2H)-pyridazinone

To 4-chloro-5-(2-chloro-4-fluorophenyl)-2-methyl-3(2H)-pyridazinone (i.e. the product of Step B) (0.25 g, 0.93 mmol) in tetrahydrofuran (1.86 mL) was added zinc chloro 2,2,6,6-tetramethylpiperidide lithium chloride complex (2.42 mL, 0.7 M in tetrahydrofuran). After 5 minutes, the reaction mixture was added via syringe to a mixture of tris(dibenzylideneacetone)-dipalladium(0) (0.17 g, 0.19 mmol), tri(2-furyl)phosphine (0.09 g, 0.37 mmol) and iodobenzene (0.38 g, 1.86 mmol) in tetrahydrofuran (1.5 mL). After stirring for 16 h, the reaction mixture was partitioned between ethyl acetate and water. The layers were separated, and the aqueous layer was extracted with ethyl acetate. The combined organics were dried over magnesium sulfate, filtered and concentrated under reduced pressure. The resulting material was purified by silica gel column chromatography (eluting with a gradient of 0 to 40% ethyl acetate in hexanes) to provide the title compound, a compound of the present invention, as a solid (0.26 g).

¹H NMR (CDCl₃) δ 7.3-7.26 (m, 1H), 7.24-7.21 (m, 2H), 7.17-7.13 (m, 3H), 7.06-7.03 (m, 1H), 6.99-6.95 (m, 1H), 3.98 (s, 3H).

### EXAMPLE 17

### Preparation of 5-(2-chloro-4-fluorophenyl)-4-methoxy-2-methyl-6-phenyl-3(2H)-pyridazinone (Compound 142)

To a mixture of 4-chloro-5-(2-chloro-4-fluorophenyl)-2-methyl-6-phenyl-3(2H)-pyridazinone (i.e. the product of Example 16) (0.2 g, 0.57 mmol) in toluene (5.7 mL) was added sodium methoxide (1.38 mL, 0.5 M solution in methanol). After 3 h, more sodium methoxide (1.38 mL, 0.5 M of solution in methanol) was added to the reaction mixture and stirring was continued for an additional 2 h. The reaction mixture was concentrated under reduced pressure, and the resulting material was purified by silica gel column chromatography (eluting with a gradient of 10 to 60% ethyl acetate in hexanes) to provide the title compound, a compound of the present invention, as a solid (90 mg).

¹H NMR (CDCl₃) δ 7.25-7.18 (m, 3H), 7.15-7.10 (m, 3H), 6.98-6.95 (m, 1H), 6.91-6.87 (m, 1H), 4.13 (s, 3H), 3.91 (s, 3H).

### Formulation/Utility

A compound of Formula 1 of this invention (including N-oxides and salts thereof) will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and | 0.001-90 | 0-99.999 | 0-15 |
| Water-soluble Granules, | | | |
| Tablets and Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pp 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

One embodiment of the present invention relates to a method for controlling fungal pathogens, comprising diluting the fungicidal composition of the present invention (a compound of Formula 1 formulated with surfactants, solid diluents and liquid diluents or a formulated mixture of a compound of Formula 1 and at least one other fungicide) with water, and optionally adding an adjuvant to form a diluted composition, and contacting the fungal pathogen or its environment with an effective amount of said diluted composition.

Although a spray composition formed by diluting with water a sufficient concentration of the present fungicidal composition can provide sufficient efficacy for controlling fungal pathogens, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be anionic or nonionic surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

The amount of adjuvants added to spray mixtures is generally in the range of about 0.1% to 2.5% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1 and a film former or adhesive agent. Seeds can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

For further information regarding the art of formulation, see T. S. Woods, “The Formulator's Toolbox—Product Forms for Modern Agriculture” in Pesticide Chemistry and Bioscience, The Food—Environment Challenge, T. Brooks and T. R. Roberts, Eds., Proceedings of the 9th International Congress on Pesticide Chemistry, The Royal Society of Chemistry, Cambridge, 1999, pp. 120-133. Also see U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Active ingredient refers to the compounds in Index Tables A-L disclosed herein. Without further elaboration, it is believed that one skilled in the art using the preceding description can utilize the present invention to its fullest extent. The following Examples are, therefore, to be constructed as merely illustrative, and not limiting of the disclosure in any way whatsoever.

Example A

| High Strength Concentrate | |
|---|---|
| Compound 25 | 98.5% |
| silica aerogel | 0.5% |
| synthetic amorphous fine silica | 1.0% |

Example B

| | |
|---|---|
| Wettable Powder | |
| Compound 35 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

Example C

| | |
|---|---|
| Granule | |
| Compound 44 | 10.0% |
| attapulgite granules (low volatile matter, | 90.0% |
| 0.71/0.30 mm; U.S.S. No. 25-50 sieves) | |

Example D

| | |
|---|---|
| Extruded Pellet | |
| Compound 35 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

Example E

| | |
|---|---|
| Emulsifiable Concentrate | |
| Compound 57 | 10.0% |
| polyoxyethylene sorbitol hexoleate | 20.0% |
| C₆-C₁₀ fatty acid methyl ester | 70.0% |

Example F

| | |
|---|---|
| Microemulsion | |
| Compound 58 | 5.0% |
| polyvinylpyrrolidone-vinyl acetate copolymer | 30.0% |
| alkylpolyglycoside | 30.0% |
| glyceryl monooleate | 15.0% |
| water | 20.0% |

Example G

| | |
|---|---|
| Seed Treatment | |
| Compound 64 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Example H

| | |
|---|---|
| Fertilizer Stick | |
| Compound 66 | 2.50% |
| pyrrolidone-styrene copolymer | 4.80% |
| tristyrylphenyl 16-ethoxylate | 2.30% |
| talc | 0.80% |
| corn starch | 5.00% |
| slow-release fertilizer | 36.00% |
| kaolin | 38.00% |
| water | 10.60% |

Example I

| | |
|---|---|
| Suspension Concentrate | |
| Compound 68 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

Example J

| | |
|---|---|
| Emulsion in Water | |
| Compound 77 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

Example K

| | |
|---|---|
| Oil Dispersion | |
| Compound 80 | 25% |
| polyoxyethylene sorbitol hexaoleate | 15% |
| organically modified bentonite clay | 2.5% |
| fatty acid methyl ester | 57.5% |

Example L

| | |
|---|---|
| Suspoemulsion | |
| Compound 83 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Water-soluble and water-dispersible formulations are typically diluted with water to form aqueous compositions before application. Aqueous compositions for direct applications to the plant or portion thereof (e.g., spray tank compositions) typically contain at least about 1 ppm or more (e.g., from 1 ppm to 100 ppm) of the compound(s) of this invention.

Seed is normally treated at a rate of from about 0.001 g (more typically about 0.1 g) to about 10 g per kilogram of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Ascomycota, Basidiomycota, Zygomycota phyla, and the fungal-like Oomycota class. They are effective in controlling a broad spectrum of plant diseases, particularly foliar pathogens of ornamental, turf, vegetable, field, cereal, and fruit crops. These pathogens include but are not limited to those listed in Table 1-1. For Ascomycetes and Basidiomycetes, names for both the sexual/teleomorph/perfect stage as well as names for the asexual/anamorph/imperfect stage (in parentheses) are listed where known. Synonymous names for pathogens are indicated by an equal sign. For example, the sexual/teleomorph/perfect stage name Phaeosphaeria nodorum is followed by the corresponding asexual/anamorph/imperfect stage name Stagnospora nodorum and the synonymous older name Septoria nodorum.

TABLE 1-1
Ascomycetes in the order Pleosporales including Alternaria solani, A. alternata and A. brassicae,
Guignardia bidwellii, Venturia inaequalis, Pyrenophora tritici-repentis (Dreschlera tritici-repentis =
Helminthosporium tritici-repentis) and Pyrenophora teres (Dreschlera teres = Helminthosporium
teres), Corynespora cassiicola, Phaeosphaeria nodorum (Stagonospora nodorum = Septaria
nodorum), Cochliobolus carbonum and C. heterostrophus, Leptosphaeria biglobosa
and L. maculans;
Ascomycetes in the order Mycosphaerellales including Mycosphaerella graminicola (Zymoseptoria
tritici = Septaria tritici), M. berkeleyi (Cercosporidium personatum), M. arachidis (Cercospora
arachidicola), Passalora sojina (Cercospora sojina), Cercospora zeae-maydis and C. beticola;
Ascomycetes in the order Erysiphales (the powdery mildews) such as Blumeria graminis f.sp. tritici
and Blumeria graminis f.sp. hordei, Erysiphe polygoni, E. necator (= Uncinula necator),
Podosphaera fuliginea (= Sphaerotheca fuliginea), and Podosphaera leucotricha
(= Sphaerotheca fuliginea);
Ascomycetes in the order Helotiales such as Botryotinia fuckeliana (Botrytis cinerea), Oculimacula
yallundae (= Tapesia yallundae; anamorph Helgardia herpotrichoides = Pseudocercosporella
herpetrichoides), Monilinia fructicola, Sclerotinia sclerotiorum, Sclerotinia minor,
and Sclerotinia homoeocarpa;
Ascomycetes in the order Hypocreales such as Giberella zeae (Fusarium graminearum),
G. monoliformis (Fusarium moniliforme), Fusarium solani (= Neocosmopora solani) and
Verticillium dahliae;
Ascomycetes in the order Eurotiales such as Aspergillus flavus and A. parasiticus;
Ascomycetes in the order Diaporthales such as Cryptosphorella viticola (= Phomopsis viticola),
Phomopsis longicolla, and Diaporthe phaseolorum;
Other Ascomycete pathogens including Magnaporthe grisea, Gaeumannomyces graminis,
Rhynchosporium secalis, and anthracnose pathogens such as Glomerella acutata (Colletotrichum
acutatum), G. graminicola (C. graminicola) and G. lagenaria (C. orbiculare);
Basidiomycetes in the order Urediniales (the rusts) including Puccinia recondita, P. striiformis,
Puccinia hordei, P. graminis and P. arachidis), Hemileia vastatrix and Phakopsora pachyrhizi;
Basidiomycetes in the order Ceratobasidiales such as Thanatophorum cucumeris (Rhizoctonia solani)
and Ceratobasidium oryzae-sativae (Rhizoctonia oryzae);
Basidiomycetes in the order Polyporales such as Athelia rolfsii (Sclerotium rolfsii);
Basidiomycetes in the order Ustilaginales such as Ustilago maydis;
Zygomycetes in the order Mucorales such as Rhizopus stolonifer;
Oomycetes in the order Pythiales, including Phytophthora infestans, P. megasperma, P. parasitica,
P. sojae, P. cinnamomi and P. capsid, and Pythium pathogens such as Pythium aphanidermatum,
P. graminicola, P. irregulare, P. ultimum and P. dissoticum;
Oomycetes in the order Peronosporales such as Plasmopara viticola, P. halstedii, Peronospora
hyoscyami (= Peronospora tabacina), P. manshurica, Hyaloperonospora parasitica
(= Peronospora parasitica), Pseudoperonospora cubensis and Bremia lactucae;
and other genera and species closely related to all of the above pathogens.

In addition to their fungicidal activity, the compositions or combinations also have activity against bacteria such as Erwinia amylovora, Xanthomonas campestris, Pseudomonas syringae, and other related species. By controlling harmful microorganisms, the compounds of the invention are useful for improving (i.e. increasing) the ratio of beneficial to harmful microorganisms in contact with crop plants or their propagules (e.g., seeds, corms, bulbs, tubers, cuttings) or in the agronomic environment of the crop plants or their propagules.

Compounds of the invention are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant cultivars which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance.

Treatment of genetically modified plants and seeds with compounds of the invention may result in super-additive or enhanced effects. For example, reduction in application rates, broadening of the activity spectrum, increased tolerance to biotic/abiotic stresses or enhanced storage stability may be greater than expected from just simple additive effects of the application of compounds of the invention on genetically modified plants and seeds.

Compounds of this invention are useful in seed treatments for protecting seeds from plant diseases. In the context of the present disclosure and claims, treating a seed means contacting the seed with a biologically effective amount of a compound of this invention, which is typically formulated as a composition of the invention. This seed treatment protects the seed from soil-borne disease pathogens and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

Compounds of this invention and their compositions, both alone and in combination with other fungicides, nematicides and insecticides, are particularly useful in seed treatment for crops including, but not limited to, maize or corn, soybeans, cotton, cereal (e.g., wheat, oats, barley, rye and rice), potatoes, vegetables and oilseed rape.

Furthermore, the compounds of this invention are useful in treating postharvest diseases of fruits and vegetables caused by fungi, oomycetes and bacteria. These infections can occur before, during and after harvest. For example, infections can occur before harvest and then remain dormant until some point during ripening (e.g., host begins tissue changes in such a way that infection can progress or conditions become conducive for disease development); also infections can arise from surface wounds created by mechanical or insect injury. In this respect, the compounds of this invention can reduce losses (i.e. losses resulting from quantity and quality) due to postharvest diseases which may occur at any time from harvest to consumption. Treatment of postharvest diseases with compounds of the invention can increase the period of time during which perishable edible plant parts (e.g., fruits, seeds, foliage, stems, bulbs, tubers) can be stored refrigerated or un-refrigerated after harvest, and remain edible and free from noticeable or harmful degradation or contamination by fungi or other microorganisms. Treatment of edible plant parts before or after harvest with compounds of the invention can also decrease the formation of toxic metabolites of fungi or other microorganisms, for example, mycotoxins such as aflatoxins.

Plant disease control is ordinarily accomplished by applying an effective amount of a compound of this invention either pre- or post-infection, to the portion of the plant to be protected such as the roots, stems, foliage, fruits, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants. Control of postharvest pathogens which infect the produce before harvest is typically accomplished by field application of a compound of this invention, and in cases where infection occurs after harvest the compounds can be applied to the harvested crop as dips, sprays, fumigants, treated wraps and box liners.

The compounds can also be applied using an unmanned aerial vehicle (UAV) for the dispension of the compositions disclosed herein over a planted area. In some embodiments the planted area is a crop-containing area. In some embodiments, the crop is selected from a monocot or dicot. In some embodiments, the crop is selected form rice, corn, barley, soybean, wheat, vegetable, tobacco, tea tree, fruit tree and sugar cane. In some embodiments, the compositions disclosed herein are formulated for spraying at an ultra-low volume. Products applied by drones may use water or oil as the spray carrier. Typical spray volume (including product) used for drone applications globally is 5.0 liters/ha-100 liters/ha (approximately 0.5-10 gpa). This includes the range of ultra low spray volume (ULV) to low spray volume (LV). Although not common there may be situations where even lower spray volumes could be used as low as 1.0 liter/ha (0.1 gpa).

Rates of application for these compounds (i.e. a fungicidally effective amount) can be influenced by factors such as the plant diseases to be controlled, the plant species to be protected, the population structure of the pathogen to be controlled, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.001 g (more typically about 0.1 g) to about 10 g per kilogram of seed.

Compounds of the present invention may also be useful for increasing vigor of a crop plant. This method comprises contacting the crop plant (e.g., foliage, flowers, fruit or roots) or the seed from which the crop plant is grown with a compound of Formula 1 in amount sufficient to achieve the desired plant vigor effect (i.e. biologically effective amount). Typically the compound of Formula 1 is applied in a formulated composition. Although the compound of Formula 1 is often applied directly to the crop plant or its seed, it can also be applied to the locus of the crop plant, i.e. the environment of the crop plant, particularly the portion of the environment in close enough proximity to allow the compound of Formula 1 to migrate to the crop plant. The locus relevant to this method most commonly comprises the growth medium (i.e. medium providing nutrients to the plant), typically soil in which the plant is grown. Treatment of a crop plant to increase vigor of the crop plant thus comprises contacting the crop plant, the seed from which the crop plant is grown or the locus of the crop plant with a biologically effective amount of a compound of Formula 1.

Increased crop vigor can result in one or more of the following observed effects: (a) optimal crop establishment as demonstrated by excellent seed germination, crop emergence and crop stand; (b) enhanced crop growth as demonstrated by rapid and robust leaf growth (e.g., measured by leaf area index), plant height, number of tillers (e.g., for rice), root mass and overall dry weight of vegetative mass of the crop; (c) improved crop yields, as demonstrated by time to flowering, duration of flowering, number of flowers, total biomass accumulation (i.e. yield quantity) and/or fruit or grain grade marketability of produce (i.e. yield quality); (d) enhanced ability of the crop to withstand or prevent plant disease infections and arthropod, nematode or mollusk pest infestations; and (e) increased ability of the crop to withstand environmental stresses such as exposure to thermal extremes, suboptimal moisture or phytotoxic chemicals.

The compounds of the present invention may increase the vigor of treated plants compared to untreated plants by preventing and/or curing plant diseases caused by fungal plant pathogens in the environment of the plants. In the absence of such control of plant diseases, the diseases reduce plant vigor by consuming plant tissues or sap, or transmitting plant pathogens such as viruses. Even in the absence of fungal plant pathogens, the compounds of the invention may increase plant vigor by modifying metabolism of plants. Generally, the vigor of a crop plant will be most significantly increased by treating the plant with a compound of the invention if the plant is grown in a nonideal environment, i.e. an environment comprising one or more aspects adverse to the plant achieving the full genetic potential it would exhibit in an ideal environment.

Of note is a method for increasing vigor of a crop plant wherein the crop plant is grown in an environment comprising plant diseases caused by fungal plant pathogens. Also of note is a method for increasing vigor of a crop plant wherein the crop plant is grown in an environment not comprising plant diseases caused by fungal plant pathogens. Also of note is a method for increasing vigor of a crop plant wherein the crop plant is grown in an environment comprising an amount of moisture less than ideal for supporting growth of the crop plant.

Compounds of this invention can also be mixed with one or more other biologically active compounds or agents including fungicides, insecticides, nematicides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

As mentioned in the Summary of the Invention, one aspect of the present invention is a fungicidal composition comprising (i.e. a mixture or combination of) a compound of Formula 1, an N-oxide, or a salt thereof (i.e. component a), and at least one other fungicide (i.e. component b). Of note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a fungicidally effective amount of at least one additional fungicidal active ingredient having a similar spectrum of control but a different site of action.

Of note is a composition which in addition to the Formula 1 compound of component (a), includes as component (b) at least one fungicidal compound selected from the group consisting of the FRAC-defined mode of action (MOA) classes (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis in membranes, (I) melanin synthesis in cell wall, (P) host plant defense induction, (M) chemicals with multi-site activity, (U) unknown mode of action and (BM) biologicals with multiple modes of action.

FRAC-recognized or proposed target sites of action along with their FRAC target site codes belonging to the above MOA classes are (A1) RNA polymerase I, (A2) adenosine deaminase, (A3) DNA/RNA synthesis (proposed), (A4) DNA topoisomerase, (B1-B3) β-tubulin assembly in mitosis, (B4) cell division (proposed), (B5) delocalization of spectrin-like proteins, (B6) actin/myosin/fimbrin function, (C1) complex I NADH odxido-reductase, (C2) complex II: succinate dehydrogenase, (C3) complex III: cytochrome bc1 (ubiquinol oxidase) at Qo site, (C4) complex III: cytochrome bc1 (ubiquinone reductase) at Qi site, (C5) uncouplers of oxidative phosphorylation, (C6) inhibitors of oxidative phosphorylation, ATP synthase, (C7) ATP production (proposed), (C8) complex III: cytochrome bc1 (ubiquinone reductase) at Q_{X} (unknown) site, (D1) methionine biosynthesis (proposed), (D2-D5) protein synthesis, (E1) signal transduction (mechanism unknown), (E2-E3) MAP/histidine kinase in osmotic signal transduction, (F2) phospholipid biosynthesis, methyl transferase, (F3) lipid peroxidation (proposed), (F4) cell membrane permeability, fatty acids (proposed), (F6) microbial disrupters of pathogen cell membranes, (F7) cell membrane disruption (proposed), (G1) C14- demethylase in sterol biosynthesis, (G2) Δ14-reductase and Δ8→Δ7-isomerase in sterol biosynthesis, (G3) 3-keto reductase, C4-demethylation, (G4) squalene epoxidase in sterol biosynthesis, (H3) trehalase and inositol biosynthesis, (H4) chitin synthase, (H5) cellulose synthase, (I1) reductase in melanin biosynthesis and (I2) dehydratase in melanin biosynthesis, (I3) polyketide synthase in melanin biosynthesis, (BM01) plant extract, and (BM02) microbial, living microbes or extract, metabolites.

Of particular note is a composition which in addition to the Formula 1 compound of component (a), includes as component (b) at least one fungicidal compound selected from the group consisting of the classes (b1) methyl benzimidazole carbamate (MBC) fungicides; (b2) dicarboximide fungicides; (b3) demethylation inhibitor (DMI) fungicides; (b4) phenylamide fungicides; (b5) amine/morpholine fungicides; (b6) phospholipid biosynthesis inhibitor fungicides; (b7) succinate dehydrogenase inhibitor fungicides; (b8) hydroxy(2-amino-)pyrimidine fungicides; (b9) anilinopyrimidine fungicides; (b10) N-phenyl carbamate fungicides; (b11) quinone outside inhibitor (QoI) fungicides; (b12) phenylpyrrole fungicides; (b13) azanaphthalene fungicides; (b14) lipid peroxidation inhibitor fungicides; (b15) melanin biosynthesis inhibitor-reductase (MBI-R) fungicides; (b16) melanin biosynthesis inhibitor-dehydratase (MBI-D) fungicides; (b17) sterol biosynthesis inhibitor (SBI): Class III fungicides; (b18) squalene-epoxidase inhibitor fungicides; (b19) polyoxin fungicides; (b20) phenylurea fungicides; (b21) quinone inside inhibitor (QiI) fungicides; (b22) benzamide and thiazole carboxamide fungicides; (b23) enopyranuronic acid antibiotic fungicides; (b24) hexopyranosyl antibiotic fungicides; (b25) glucopyranosyl antibiotic: protein synthesis fungicides; (b26) glucopyranosyl antibiotic: trehalase and inositol biosynthesis fungicides; (b27) cyanoacetamideoxime fungicides; (b28) carbamate fungicides; (b29) oxidative phosphorylation uncoupling fungicides; (b30) organo tin fungicides; (b31) carboxylic acid fungicides; (b32) heteroaromatic fungicides; (b33) phosphonate fungicides; (b34) phthalamic acid fungicides; (b35) benzotriazine fungicides; (b36) benzene-sulfonamide fungicides; (b37) pyridazinone fungicides; (b38) thiophene-carboxamide fungicides; (b39) complex I NADH oxidoreductase inhibitor fungicides; (b40) carboxylic acid amide (CAA) fungicides; (b41) tetracycline antibiotic fungicides; (b42) thiocarbamate fungicides; (b43) benzamide fungicides; (b44) microbial fungicides; (b45) Q_{X}I fungicides; (b46) plant extract fungicides; (b47) host plant defense induction fungicides; (b48) multi-site contact activity fungicides; (b49) fungicides other than fungicides of classes (b1) through (b48); and salts of compounds of classes (b1) through (b48).

Further descriptions of these classes of fungicidal compounds are provided below.

(b1) “Methyl benzimidazole carbamate (MBC) fungicides” (FRAC code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(b2) “Dicarboximide fungicides” (FRAC code 2) inhibit a MAP/histidine kinase in osmotic signal transduction. Examples include chlozolinate, iprodione, procymidone and vinclozolin.

(b3) “Demethylation inhibitor (DMI) fungicides” (FRAC code 3) (Sterol Biosynthesis Inhibitors (SBI): Class I) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines, pyridines and triazolinthiones. The triazoles include azaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, mefentrifluconazole, metconazole, myclobutanil, penconazole, propiconazole, quinconazole, simeconazole, tebuconazole, tetraconazole, triadimefon, triadimenol, triticonazole, uniconazole, uniconazole-P, α-(1-chlorocyclopropyl)-α-[2-(2,2-dichlorocyclopropyl)ethyl]-1H-1,2,4-triazole-1-ethanol, rel-1- [[(2R,3S)-3-(2-chloro-phenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole. The imidazoles include econazole, imazalil, oxpoconazole, prochloraz, pefurazoate and triflumizole. The pyrimidines include fenarimol, nuarimol and triarimol. The piperazines include triforine. The pyridines include buthiobate, pyrifenox, pyrisoxazole (3-[(3R)-5-(4-chlorophenyl)-2,3-dimethyl3-isoxazolidinyl]pyridine, mixture of 3R,5R- and 3R,5S-isomers) and (αS)-[3-(4-chloro-2-fluorophenyl)5-(2,4-difluorophenyl)-4-isoxazolyl]-3-pyridinemethanol. The triazolinthiones include prothioconazole and 2-[2-(1-chlorocyclopropyl)-4-(2,2-dichlorocyclopropyl)2-hydroxybutyl]-1,2-dihydro-3H-1,2,4-triazole-3-thione. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(b4) “Phenylamide fungicides” (FRAC code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M (also known as kiralaxyl), furalaxyl, metalaxyl and metalaxyl-M (also known as mefenoxam). The oxazolidinones include oxadixyl. The butyrolactones include ofurace.

(b5) “Amine/morpholine fungicides” (FRAC code 5) (SBI: Class II) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(b6) “Phospholipid biosynthesis inhibitor fungicides” (FRAC code 6) inhibit growth of fungi by affecting phospholipid biosynthesis. Phospholipid biosynthesis fungicides include phophorothiolate and dithiolane fungicides. The phosphorothiolates include edifenphos, iprobenfos and pyrazophos. The dithiolanes include isoprothiolane.

(b7) “Succinate dehydrogenase inhibitor (SDHI) fungicides” (FRAC code 7) inhibit Complex II fungal respiration by disrupting a key enzyme in the Krebs Cycle (TCA cycle) named succinate dehydrogenase. Inhibiting respiration prevents the fungus from making ATP, and thus inhibits growth and reproduction. SDHI fungicides include phenylbenzamide, furan carboxamide, oxathiin carboxamide, thiazole carboxamide, pyrazole-4-carboxamide, pyridine carboxamide, phenyl oxoethyl thiophene amides and pyridinylethyl benzamides. The benzamides include benodanil, flutolanil and mepronil. The furan carboxamides include fenfuram. The oxathiin carboxamides include carboxin and oxycarboxin. The thiazole carboxamides include thifluzamide. The pyrazole-4-carboxamides include benzovindiflupyr (N-[9-(dichloro-methylene)-1,2,3,4-tetrahydro-1,4-methanonaphthalen-5-yl]-3-(difluoromethyl)-1-methyl-1H-pyrazole-4-carboxamide), bixafen, fluindapyr, fluxapyroxad (3-(difluoromethyl)-1-methyl-N-(3′,4′,5′-trifluoro[1,1′-biphenyl]-2-yl)-1H-pyrazole-4-carboxamide), furametpyr, isopyrazam (3-(difluoromethyl)-1-methyl-N-[1,2,3,4-tetrahydro-9-(1-methylethyl)-1,4-methanonaphthalen yl]-1H-pyrazole-4-carboxamide), penflufen (N-[2-(1,3-dimethylbutyl)phenyl]-5-fluoro-1,3-dimethyl-1H-pyrazole-4-carboxamide), penthiopyrad, pydiflumetofen, sedaxane (N-[2-[1,1′-bicyclopropyl]-2-ylphenyl]-3-(difluoromethyl)-1-methyl-1H-pyrazole-4-carboxamide), N-[2-(1S,2R)-[1,1′-bicyclopropyl]-2-ylphenyl]-3-(difluoromethyl)-1-methyl-1H-pyrazole carboxamide, 3-(difluoromethyl)-N-(2,3-dihydro-1,1,3-trimethyl-1H-inden-4-yl)-1-methyl-1H-pyrazole-4-carboxamide, N-[2-(2,4-dichlorophenyl)₂-methoxy-1-methylethyl]-3-(difluoro-methyl)-1-methyl-1H-pyrazole-4-carboxamide and N-cyclopropyl-3-(difluoromethyl)-5-fluoro-1-methyl-N-[[2-(1-methylethyl)phenyl]methyl]-1H-pyrazole-4-carboxamide. The pyridine carboxamides include boscalid. The phenyl oxoethyl thiophene amides include isofetamid (N-[1,1-dimethyl-2-[2-methyl-4-(1-methylethoxy)phenyl]-2-oxoethyl]-3-methyl-2-thiophenecarboxamide). The pyridinylethyl benzamides include fluopyram.

(b8) “Hydroxy-(2-amino-)pyrimidine fungicides” (FRAC code 8) inhibit nucleic acid synthesis by interfering with adenosine deaminase. Examples include bupirimate, dimethirimol and ethirimol.

(b9) “Anilinopyrimidine fungicides” (FRAC code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(b10) “N-Phenyl carbamate fungicides” (FRAC code 10) inhibit mitosis by binding to β-tubulin and disrupting microtubule assembly. Inhibition of microtubule assembly can disrupt cell division, transport within the cell and cell structure. Examples include diethofencarb.

(b11) “Quinone outside inhibitor (QoI) fungicides” (FRAC code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides include methoxyacrylate, methoxycarbamate, oximinoacetate, oximinoacetamide and dihydrodioxazine fungicides (collectively also known as strobilurin fungicides), and oxazolidinedione, imidazolinone and benzylcarbamate fungicides. The methoxyacrylates include azoxystrobin, coumoxystrobin (methyl (αE)-2- [[(3-butyl-4-methyl-2-oxo-2H-1-benzopyran-7-yl)oxy]methyl]-α-(methoxy-methylene)benzeneacetate), enoxastrobin (methyl (αE)-2-[[[(E)-[(2E)-3-(4-chlorophenyl)-1-methyl-2-propen-1-ylidene]amino]oxy]methyl]-α-(methoxymethylene)benzeneaceate) (also known as enestroburin), flufenoxystrobin (methyl (αE)-2-[[2-chloro-4-(trifluoromethyl)-phenoxy]methyl]-α-(methoxymethylene)benzeneacetate), picoxystrobin, and pyraoxystrobin (methyl (αE)-2-[[[3-(4-chlorophenyl)-1-methyl-1H-pyrazol-5-yl]oxy]methyl]-α-(methoxy-methylene)benzeneacetate). The methoxycarbamates include pyraclostrobin, pyrametostrobin (methyl N-[2-[[(1,4-dimethyl-3-phenyl-1H-pyrazol-5-yl)oxy]methyl]phenyl]-N-methoxy-carbamate) and triclopyricarb (methyl N-methoxy-N-[2-[[(3,5,6-trichloro-2-pyridinyl)oxy]-methyl]phenyl]carbamate). The oximinoacetates include kresoxim-methyl and trifloxystrobin. The oximinoacetamides include dimoxystrobin, fenaminstrobin ((αE)-2-[[[(E)-[(2E)-3-(2,6-dichlorophenyl)-1-methyl-2-propen-1-ylidene]amino]oxy]methyl]-α-(methoxyimino)-N-methyl-benzeneacetamide), metominostrobin, orysastrobin and α-[methoxyimino]-N-methyl-2-[[[1-[3-(trifluoromethyl)phenyl]ethoxy]imino]methyl]benzeneacetamide. The dihydrodioxazines include fluoxastrobin. The oxazolidinediones include famoxadone. The imidazolinones include fenamidone. The benzylcarbamates include pyribencarb. Class (b11) also includes mandestrobin (2-[(2,5-dimethylphenoxy)methyl]-α-methoxy-N-benzeneacetamide).

(b12) “Phenylpyrrole fungicides” (FRAC code 12) inhibit a MAP/histidine kinase associated with osmotic signal transduction in fungi. Fenpiclonil and fludioxonil are examples of this fungicide class.

(b13) “Azanaphthalene fungicides” (FRAC code 13) are proposed to inhibit signal transduction by a mechanism which is as yet unknown. They have been shown to interfere with germination and/or appressorium formation in fungi that cause powdery mildew diseases. Azanaphthalene fungicides include aryloxyquinolines and quinazolinones. The aryloxyquinolines include quinoxyfen. The quinazolinones include proquinazid.

(b14) “Lipid peroxidation inhibitor fungicides” (FRAC code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic hydrocarbon and 1,2,4-thiadiazole fungicides. The aromatic hydrocarboncarbon fungicides include biphenyl, chloroneb, dicloran, quintozene, tecnazene and tolclofos-methyl. The 1,2,4-thiadiazoles include etridiazole.

(b15) “Melanin biosynthesis inhibitors-reductase (MBI-R) fungicides” (FRAC code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(b16) “Melanin biosynthesis inhibitors-dehydratase (MBI-D) fungicides” (FRAC code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(b17) “Sterol Biosynthesis Inhibitor (SBI): Class III fungicides (FRAC code 17) inhibit 3-ketoreductase during C4-demethylation in sterol production. SBI: Class III inhibitors include hydroxyanilide fungicides and amino-pyrazolinone fungicides. Hydroxyanilides include fenhexamid. Amino-pyrazolinones include fenpyrazamine (S-2-propen-1-yl 5-amino-2,3-di-hydro-2-(1-methylethyl)-4-(2-methylphenyl)-3-oxo-1H-pyrazole-1-carbothioate).

(b18) “Squalene-epoxidase inhibitor fungicides” (FRAC code 18) (SBI: Class IV) inhibit squalene-epoxidase in the sterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(b19) “Polyoxin fungicides” (FRAC code 19) inhibit chitin synthase. Examples include polyoxin.

(b20) “Phenylurea fungicides” (FRAC code 20) are proposed to affect cell division. Examples include pencycuron.

(b21) “Quinone inside inhibitor (QiI) fungicides” (FRAC code 21) inhibit Complex III mitochondrial respiration in fungi by affecting ubiquinone reductase. Reduction of ubiquinone is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(b22) “Benzamide and thiazole carboxamide fungicides” (FRAC code 22) inhibit mitosis by binding to β-tubulin and disrupting microtubule assembly. Inhibition of microtubule assembly can disrupt cell division, transport within the cell and cell structure. The benzamides include zoxamide. The thiazole carboxamides include ethaboxam.

(b23) “Enopyranuronic acid antibiotic fungicides” (FRAC code 23) inhibit growth of fungi by affecting protein biosynthesis. Examples include blasticidin-S.

(b24) “Hexopyranosyl antibiotic fungicides” (FRAC code 24) inhibit growth of fungi by affecting protein biosynthesis. Examples include kasugamycin.

(b25) “Glucopyranosyl antibiotic: protein synthesis fungicides” (FRAC code 25) inhibit growth of fungi by affecting protein biosynthesis. Examples include streptomycin.

(b26) “Glucopyranosyl antibiotic: trehalase and inositol biosynthesis fungicides” (FRAC code 26) inhibit trehalase and inositol biosynthesis. Examples include validamycin.

(b27) “Cyanoacetamideoxime fungicides (FRAC code 27) include cymoxanil.

(b28) “Carbamate fungicides” (FRAC code 28) are considered multi-site inhibitors of fungal growth. They are proposed to interfere with the synthesis of fatty acids in cell membranes, which then disrupts cell membrane permeability. Propamacarb, iodocarb, and prothiocarb are examples of this fungicide class.

(b29) “Oxidative phosphorylation uncoupling fungicides” (FRAC code 29) inhibit fungal respiration by uncoupling oxidative phosphorylation. Inhibiting respiration prevents normal fungal growth and development. This class includes 2,6-dinitroanilines such as fluazinam, and dinitrophenyl crotonates such as dinocap, meptyldinocap and binapacryl.

(b30) “Organo tin fungicides” (FRAC code 30) inhibit adenosine triphosphate (ATP) synthase in oxidative phosphorylation pathway. Examples include fentin acetate, fentin chloride and fentin hydroxide.

(b31) “Carboxylic acid fungicides” (FRAC code 31) inhibit growth of fungi by affecting deoxyribonucleic acid (DNA) topoisomerase type II (gyrase). Examples include oxolinic acid.

(b32) “Heteroaromatic fungicides” (Fungicide Resistance Action Committee (FRAC) code 32) are proposed to affect DNA/ribonucleic acid (RNA) synthesis. Heteroaromatic fungicides include isoxazoles and isothiazolones. The isoxazoles include hymexazole and the isothiazolones include octhilinone.

(b33) “Phosphonate fungicides” (FRAC code 33) include phosphorous acid and its various salts, including fosetyl-aluminum.

(b34) “Phthalamic acid fungicides” (FRAC code 34) include teclofthalam.

(b35) “Benzotriazine fungicides” (FRAC code 35) include triazoxide.

(b36) “Benzene-sulfonamide fungicides” (FRAC code 36) include flusulfamide.

(b37) “Pyridazinone fungicides” (FRAC code 37) include diclomezine.

(b38) “Thiophene-carboxamide fungicides” (FRAC code 38) are proposed to affect ATP production. Examples include silthiofam.

(b39) “Complex I NADH oxidoreductase inhibitor fungicides” (FRAC code 39) inhibit electron transport in mitochondria and include pyrimidinamines such as diflumetorim, and pyrazole-5-carboxamides such as tolfenpyrad.

(b40) “Carboxylic acid amide (CAA) fungicides” (FRAC code 40) inhibit cellulose synthase which prevents growth and leads to death of the target fungus. Carboxylic acid amide fungicides include cinnamic acid amide, valinamide and other carbamate, and mandelic acid amide fungicides. The cinnamic acid amides include dimethomorph, flumorph and pyrimorph (3-(2-chloro-4-pyridinyl)-3-[4-(1,1-dimethylethyl)phenyl]-1-(4-morpholinyl)-2-propene-1-one). The valinamide and other carbamates include benthiavalicarb, benthiavalicarb-isopropyl, iprovalicarb, tolprocarb (2,2,2-trifluoroethyl N-[(1S)-2-methyl-1-[[(4-methylbenzoyl)amino]methyl]propyl]-carbamate) and valifenalate (methyl N-[(1-methylethoxy)carbonyl]-L-valyl-3-(4-chlorophenyl)-β-alaninate) (also known as valiphenal). The mandelic acid amides include mandipropamid, N-[2-[4- [[3-(4-chlorophenyl)-2-propyn-1-yl]oxy]-3-methoxyphenyl]ethyl]-3-methyl-2-[methylsulfonyl)amino]butanamide and N-[2-[4- [[3-(4-chlorophenyl)-2-propyn-1-yl]oxy]-3-methoxyphenyl]ethyl]-3-methyl-2-[(ethylsulfonyl)amino]butanamide.

(b41) “Tetracycline antibiotic fungicides” (FRAC code 41) inhibit growth of fungi by affecting protein synthesis. Examples include oxytetracycline.

(b42) “Thiocarbamate fungicides” (FRAC code 42) include methasulfocarb.

(b43) “Benzamide fungicides” (FRAC code 43) inhibit growth of fungi by delocalization of spectrin-like proteins. Examples include pyridinylmethyl benzamide fungicides such as fluopicolide (now FRAC code 7, pyridinylethyl benzamides).

(b44) “Microbial fungicides” (FRAC code 44) disrupt fungal pathogen cell membranes. Microbial fungicides include Bacillus species such as Bacillus amyloliquefaciens strains QST 713, FZB24, MB1600, D747 and the fungicidal lipopeptides which they produce.

(b45) “Q_{X}I fungicides” (FRAC code 45) inhibit Complex III mitochondrial respiration in fungi by affecting ubiquinone reductase at an unknown (Q_{X}) site of the cytochrome bc₁ complex. Inhibiting mitochondrial respiration prevents normal fungal growth and development. Q_{X}I fungicides include triazolopyrimidylamines such as ametoctradin (5-ethyl-6-octyl[1,2,4]triazolo[1,5-α]pyrimidin-7-amine).

(b46) “Plant extract fungicides” are proposed to act by cell membrane disruption. Plant extract fungicides include terpene hydrocarbons and terpene alcohols such as the extract from Melaleuca alternifolia (tea tree).

(b47) “Host plant defense induction fungicides” (FRAC code P) induce host plant defense mechanisms. Host plant defense induction fungicides include benzothiadiazoles, benzisothiazole and thiadiazole-carboxamide fungicides. The benzothiadiazoles include acibenzolar-S-methyl. The benzisothiazoles include probenazole. The thiadiazole-carboxamides include tiadinil and isotianil.

(b48) “Multi-site contact fungicides” inhibit fungal growth through multiple sites of action and have contact/preventive activity. This class of fungicides includes: (b48.1) “copper fungicides” (FRAC code M1)”, (b48.2) “sulfur fungicides” (FRAC code M2), (b48.3) “dithiocarbamate fungicides” (FRAC code M3), (b48.4) “phthalimide fungicides” (FRAC code M4), (b48.5) “chloronitrile fungicides” (FRAC code M5), (b48.6) “sulfamide fungicides” (FRAC code M6), (b48.7) multi-site contact “guanidine fungicides” (FRAC code M7), (b48.8) “triazine fungicides” (FRAC code M8), (b48.9) “quinone fungicides” (FRAC code M9), (b48.10) “quinoxaline fungicides” (FRAC code M10) and (b48.11) “maleimide fungicides” (FRAC code M11). “Copper fungicides” are inorganic compounds containing copper, typically in the copper (II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. Multi-site contact “guanidine fungicides” include, guazatine, iminoctadine albesilate and iminoctadine triacetate. “Triazine fungicides” include anilazine. “Quinone fungicides” include dithianon. “Quinoxaline fungicides” include quinomethionate (also known as chinomethionate). “Maleimide fungicides” include fluoroimide.

(b49) “Fungicides other than fungicides of classes (b1) through (b48)” include certain fungicides whose mode of action may be unknown. These include: (b49.1), “phenyl-acetamide fungicides” (FRAC code U6), (b49.2) “aryl-phenyl-ketone fungicides” (FRAC code U8), (b49.3) “guanidine fungicides” (FRAC code U12), (b49.4) “thiazolidine fungicides” (FRAC code U13), (b49.5) “pyrimidinone-hydrazone fungicides” (FRAC code U14) and (b49.6) compounds that bind to oxysterol-binding protein as described in PCT Patent Publication WO 2013/009971. The phenyl-acetamides include cyflufenamid and N-[[(cyclopropylmethoxy)amino][6-(difluoromethoxy)-2,3-difluorophenyl]-methylene]benzeneacetamide. The aryl-phenyl ketones include benzophenones such as metrafenone, and benzoylpyridines such as pyriofenone (5-chloro-2-methoxy-4-methyl-3-pyridinyl)(2,3,4-trimethoxy-6-methylphenyl)methanone). The quanidines include dodine. The thiazolidines include flutianil ((2Z)-2-[[2-fluoro-5-(trifluoromethyl)phenyl]thio]-2-[3-(2-methoxyphenyl)-2-thiazolidinylidene]acetonitrile). The pyrimidinonehydrazones include ferimzone. The (b49.6) class includes oxathiapiprolin (1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone) and its R-enantiomer which is 1-[4-[4-[5R-(2,6-difluorophenyl)-4,5-dihydro-3-isoxazolyl]-2-thiazolyl]-1-piperidinyl]-2-[5-methyl-3-(trifluoro-methyl)-1H-pyrazol-1-yl]ethanone (Registry Number 1003319-79-6). The (b49) class also includes bethoxazin, flometoquin (2-ethyl-3,7-dimethyl-6-[4-(trifluoromethoxy)phenoxy]-4-quinolinyl methyl carbonate), fluoroimide, neo-asozin (ferric methanearsonate), picarbutrazox (1,1-dimethylethyl N-[6- [[[[((Z)1-methyl-1H-tetrazol-5-yl)phenylmethylene]amino]oxy]-methyl]-2-pyridinyl]carbamate), pyrrolnitrin, quinomethionate, tebufloquin (6-(1,1-dimethylethyl)-8-fluoro-2,3-dimethyl-4-quinolinyl acetate), tolnifanide (N-(4-chloro-2-nitro-phenyl)-N-ethyl-4-methylbenzenesulfonamide), 2-butoxy-6-iodo-3-propyl-4H-1-benzopyran one, 3-butyn-1-yl, N-[6- [[[[(1-methyl-1H-tetrazol-5-yl)phenylmethylene]amino]oxy]methyl]-2-pyridinyl]carbamate, (N-(4-chloro-2-nitrophenyl)-N-ethyl-4-methylbenzene sulfonamide), N′-[4-[4-chloro-3-(trifluoromethyl)phenoxy]-2,5-dimethylphenyl]-N-ethyl-N-methylmethanimid-amide, N-[[(cyclopropylmethoxy)amino][6-(difluoromethoxy)-2,3-difluorophenyl]methylene]-benzeneacetamide, 2,6-dimethyl-1H,5H-[1,4]dithiino[2,3-c:5,6-c′]dipyrrole-1,3,5,7(2H,6H)-tetrone, 5-fluoro-2-[(4-methylphenyl)methoxy]-4-pyrimidinamine, 5-fluoro-2-[(4-fluoro-phenyl)methoxy]-4-pyrimidinamine and 4-fluorophenyl N-[1-[[[1-(4-cyanophenyl)ethyl]-sulfonyl]methyl]propyl]carbamate, pentyl N-[6- [ [[ [(1-methyl-1H-tetrazol-5-yl)phenyl-methylene]amino]oxy]methyl]-2-pyridinyl]carbamate, pentyl N-[4- [ [ [[(1-methyl-1H-tetrazol-5-yl)phenylmethylene]amino]oxy]methyl]-2-thiazolyl]carbamate and pentyl N-[6- [[ [[(Z)-(1-methyl-1H-tetrazol-5-yl)phenylmethylene]amino]oxy]methyl]-2-pyridinyl]carbamate. The (b46) class further includes mitosis- and cell division-inhibiting fungicides besides those of the particular classes described above (e.g., (b1), (b10) and (b22)).

Additional “Fungicides other than fungicides of classes (1) through (46)” whose mode of action may be unknown, or may not yet be classified include a fungicidal compound selected from components (b49.7) through (b49.13), as shown below.

Component (b49.7) relates to a compound of Formula b49.7

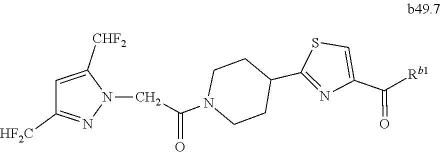

wherein R^{b1} is

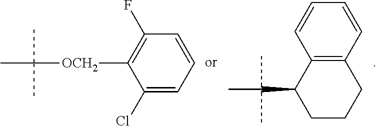

Examples of a compound of Formula b49.7 include (b49.7a) (2-chloro-6-fluorophenyl)methyl 2-[1-[2-[3,5-bis(difluoromethyl)-1H-pyrazol-1-yl]acetyl]-4-piperidinyl]-4-thiazolecarboxylate (Registry Number 1299409-40-7) and (b49.7b) (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate (Registry Number 1299409-42-9). Methods for preparing compounds of Formula b46.2 are described in PCT Patent Publications WO 2009/132785 and WO 2011/051243.

Component (b49.8) relates to a compound of Formula b49.8

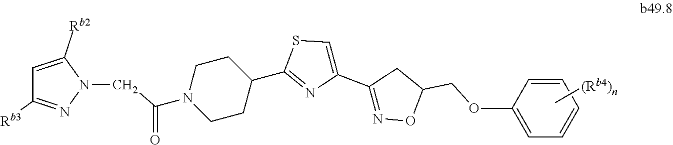

wherein R^{b2} is CH₃, CF₃ or CHF₂; R^{b3} is CH₃, CF₃ or CHF₂; R^{b4} is halogen or cyano;
and n is 0, 1, 2 or 3.
   Examples of a compound of Formula b49.8 include (b49.8a) 1-[4-[4-[5-[(2,6-difluorophenoxy)-methyl]-4,5-dihydro-3-isoxazolyl]-2-thiazolyl]-1-piperdinyl]-2-[5-methyl-3-(trifluoromethyl)-1H-pyrazol-1-yl]ethanone. Methods for preparing compounds of Formula b49.8 are described in PCT Patent Application PCT/US11/64324.

Component (b4799) relates to a compound of Formula b49.9

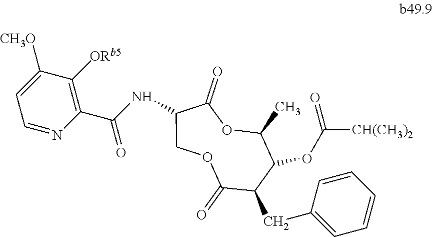

wherein R^{b5} is

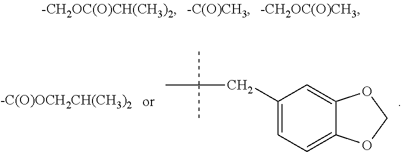

Examples of a compound of Formula b49.9 include (b49.9a) [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]-carbonyl]-3-pyridinyl]oxy]methyl 2-methylpropanoate (Registry Number 517875-34-2; common name fenpicoxamid), (b49.9b) (3S,6S,7R,8R)-3-[[[3-(acetyloxy)-4-methoxy-2-pyridinyl]-carbonyl]amino]-6-methyl-4,9-dioxo-8-(phenylmethyl)-1,5-dioxonan-7-yl 2-methylpropanoate (Registry Number 234112-93-7), (b49.9c) (3S,6S,7R,8R)-3-[[[3[(acetyloxy)methoxy]-4-methoxy-2-pyridinyl]carbonyl]amino]-6-methyl-4,9-dioxo-8-(phenylmethyl)-1,5-dioxonan-7-yl 2-methyl-propanoate (Registry Number 517875-31-9), (b49.9d) (3S,6S,7R,8R)-3-[[[4-methoxy-3-[[(2-methylpropoxy)carbonyl]oxy]-2-pyridinyl]carbonyl]amino]6-methyl-4,9-dioxo (phenylmethyl)-1,5-dioxonan-7-yl 2-methylpropanoate (Registry Number 328256-72-0), and (b49.9e) N-[[3-(1,3-benzodioxol-5-ylmethoxy)-4-methoxy-2-pyridinyl]carbonyl]-O-[2,5-dideoxy-3-O-(2-methyl-1-oxopropyl)-2-(phenylmethyl)L-arabinonoyl]-L-serine, (1→4′)-lactone (Registry Number 1285706-70-8). Methods for preparing compounds of Formula b49.9 are described in PCT Patent Publications WO 99/40081, WO 2001/014339, WO 2003/035617 and WO 2011044213.

Component (b49.10) relates to a compound of Formula b49.10

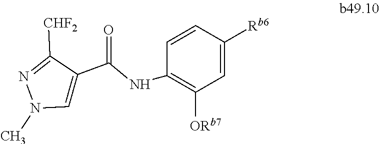

wherein R^{b6} is H or F, and R^{b7} is —CF₂CHFCF₃ or —CF₂CF₂H. Examples of a compound of Formula b49.10 are (b49.10a) 3-(difluoromethyl)-N-[4-fluoro-2-(1,1,2,3,3,3-hexafluoro-propoxy)phenyl]-1-methyl-1H-pyrazole-4-carboxamide (Registry Number 1172611-40-3) and (b49.10b) 3-(difluoromethyl)-1-methyl-N-[2-(1,1,2,2-tetrafluoroethoxy)phenyl]-1H-pyrazole4-carboxamide (Registry Number 923953-98-4). Compounds of Formula 49.10 can be prepared by methods described in PCT Patent Publication WO 2007/017450.

Component b49.11 relates a compound of Formula b49.11

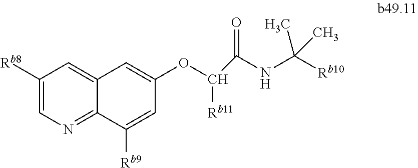

wherein
R^{b8} is halogen, C₁-C₄ alkoxy or C₂-C₄ alkynyl;
R^{b9} is H, halogen or C₁-C₄ alkyl;
R^{b10} is C₁-C₁₂ alkyl, C₁-C₁₂ haloalkyl, C₁-C₁₂ alkoxy, C₂-C₁₂ alkoxyalkyl, C₂-C₁₂ alkenyl, C₂-C₁₂ alkynyl, C₄-C₁₂ alkoxyalkenyl, C₄-C₁₂ alkoxyalkynyl, C₁-C₁₂ alkylthio or C₂-C₁₂ alkylthioalkyl;
R^{b11} is methyl or —Y^{b13}—R^{b12};
R^{b12} is C₁-C₂ alkyl; and
Y^{b13} is CH₂, O or S.
   Examples of compounds of Formula b49.11 include (b49.11a) 2-[(3-bromo-6-quinolinyl)oxy]-N-(1,1-dimethyl-2-butyn-1-yl)-2-(methylthio)acetamide, (b49.11b) 2 [(3-ethynyl-6-quinolinyl)oxy]-N-[1-(hydroxymethyl)-1-methyl-2-propyn-1-yl]-2-(methylthio)acetamide, (b49.11c) N-(1,1-dimethyl-2-butyn-1-yl)-2-[(3-ethynyl-6-quinolinyl)oxy]-2-(methylthio)acetamide, (b49.11d) 2-[(3-bromo-8-methyl-6-quinolinyl)oxy]-N-(1,1-dimethyl-2-propyn-1-yl)-2-(methylthio)acetamide and (b49.11e) 2-[(3-bromo-6-quinolinyl)oxy]-N-(1,1-dimethylethyl)-butanamide. Compounds of Formula b49.11, their use as fungicides and methods of preparation are generally known; see, for example, PCT Patent Publications WO 2004/047538, WO 2004/108663, WO 2006/058699, WO 2006/058700, WO 2008/110355, WO 2009/030469, WO 2009/049716 and WO 2009/087098.

Component 49.12 relates to N′-[4-[[3-[(4-chlorophenyl)methyl]-1,2,4-thiadiazol-5-yl]oxy]-2,5-dimethylphenyl]-N-ethyl-N-methylmethanimidamide, which is believed to inhibit C24-methyl transferase involved in the biosynthesis of sterols.

Component 49.13 relates to (1S)-2,2-bis(4-fluorophenyl)-1-methylethyl N-[[3-(acetyloxy)-4-methoxy-2-pyridinyl]carbonyl]-L-alaninate (Registry Number 1961312-55-9, common name florylpicoxamid), which is believed to be a Quinone inside inhibitor (QiI) fungicide (FRAC code 21) inhibiting the Complex III mitochondrial respiration in fungi.

Therefore of note is a mixture (i.e. composition) comprising a compound of Formula 1 and at least one fungicidal compound selected from the group consisting of the aforedescribed classes (1) through (49). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (49). Also of particular note is a composition comprising said mixture (in fungicidally effective amount) and further comprising at least one additional surfactant selected from the group consisting of surfactants, solid diluents and liquid diluents.

Examples of component (b) fungicides include acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, captafol, captan, carbendazim, carboxin, carpropamid, chloroneb, chlorothalonil, chlozolinate, clotrimazole, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dithianon, dithiolanes, dodemorph, dodine, econazole, edifenphos, enoxastrobin (also known as enestroburin), epoxiconazole, etaconazole, ethaboxam, ethirimol, etridiazole, famoxadone, fenamidone, fenarimol, fenaminstrobin, fenbuconazole, fenfuram, fenhexamid, fenoxanil, fenpiclonil, fenpropidin, fenpropimorph, fenpyrazamine, fentin acetate, fentin chloride, fentin hydroxide, ferbam, ferimzone, flometoquin, florylpicoxamid, fluazinam, fludioxonil, flufenoxystrobin, fluindapyr, flumorph, fluopicolide, fluopyram, flouroimide, fluoxastrobin, fluquinconazole, flusilazole, flusulfamide, flutianil, flutolanil, flutriafol, fluxapyroxad, folpet, fthalide, fuberidazole, furalaxyl, furametpyr, guazatine, hexaconazole, hymexazole, imazalil, imibenconazole, iminoctadine albesilate, iminoctadine triacetate, iodocarb, ipconazole, iprobenfos, iprodione, iprovalicarb, isoconazole, isofetamid, isoprothiolane, isopyrazam, isotianil, kasugamycin, kresoxim-methyl, mancozeb, mandepropamid, mandestrobin, maneb, mepanipyrim, mepronil, meptyldinocap, metalaxyl (including metalaxyl-M/mefenoxam), mefentrifluconazole, metconazole, methasulfocarb, metiram, metominostrobin, metrafenone, miconazole, myclobutanil, naftifine, neo-asozin, nuarimol, octhilinone, ofurace, orysastrobin, oxadixyl, oxathiapiprolin, oxolinic acid, oxpoconazole, oxycarboxin, oxytetracycline, pefurazoate, penconazole, pencycuron, penflufen, penthiopyrad, phosphorous acid (including salts thereof, e.g., fosetyl-aluminum), picarbutrazox, picoxystrobin, piperalin, polyoxin, probenazole, prochloraz, procymidone, propamacarb, propiconazole, propineb, proquinazid, prothiocarb, prothioconazole, pyraclostrobin, pyrametostrobin, pyraoxystrobin, pyrazophos, pyribencarb, pyributicarb, pyrifenox, pyrimethanil, pyriofenone, pyrisoxazole, pyroquilon, pyrrolnitrin, quinconazole, quinomethionate, quinoxyfen, quintozene, sedaxane, silthiofam, simeconazole, spiroxamine, streptomycin, sulfur, tebuconazole, tebufloquin, teclofthalam, tecnazene, terbinafine, tetraconazole, thiabendazole, thifluzamide, thiophanate, thiophanate-methyl, thiram, tiadinil, tolclofos-methyl, tolnifanide, tolprocarb, tolyfluanid, triadimefon, triadimenol, triarimol, triticonazole, triazoxide, tribasic copper sulfate, tricyclazole, triclopyricarb, tridemorph, trifloxystrobin, triflumizole, triforine, trimorphamide, uniconazole, uniconazole-P, validamycin, valifenalate (also known as valiphenal), vinclozolin, zineb, ziram, zoxamide, (3S,6S,7R,8R)-3-[[[3- [(acetyloxy)methoxy]-4-methoxy-2-pyridinyl]carbonyl]amino]-6-methyl-4,9-dioxo-8-(phenylmethyl)-1,5-dioxonan-7-yl 2-methylpropanoate, (3S,6S,7R,8R)-3-[[[3-(acetyloxy)-4-methoxy-2-pyridinyl]carbonyl]amino]-6-methyl-4,9-dioxo-8-(phenylmethyl)-1,5-dioxonan-7-yl 2-methylpropanoate, N-[[3-(1,3-benzodioxol-5-ylmethoxy)-4-methoxy-2-pyridinyl]carbonyl]-O-[2,5-dideoxy-3-O-(2-methyl-1-oxopropyl)-2-(phenylmethyl)-L-arabinonoyl]-L-serine, (1→4′)-lactone, N-[2-(1S,2R)-[1,1′-bicyclopropyl]-2-ylphenyl]-3-(difluoromethyl)-1-methyl-1H-pyrazole-4-carboxamide, 2-[(3-bromo-6-quinolinyl)oxy]-N-(1,1-dimethyl-2-butyn-1-yl)-2-(methylthio)acetamide, 2-[(3-bromo-6-quinolinyl)oxy]-N-(1,1-dimethylethyl)butanamide, 2-[(3-bromo-8-methyl-6-quinolinyl)oxy]-N-(1,1-dimethyl-2-propyn-1-yl)-2-(methylthio)acetamide, 2-butoxy-6-iodo-3-propyl-4H-1-benzopyran-4-one, 3-butyn-1-yl N-[6- [[[[(1-methyl-1H-tetrazol-5-yl)phenylmethylene]amino]oxy]methyl]-2-pyridinyl]-carbamate, α-(1-chlorocyclopropyl)-α-[2-(2,2-dichlorocyclopropyl)ethyl]-1H-1,2,4-triazole-1-ethanol, 2-[2-(1-chlorocyclopropyl)-4-(2,2-dichlorocyclopropyl)-2-hydroxybutyl]-1,2-dihydro-3H-1,2,4-triazole-3-thione, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, rel-1-[[(2R,3S)-3-(2-chlorophenyl)-2-(2,4-difluorophenyl)-2-oxiranyl]methyl]-5-(2-propen-1-ylthio)-1H-1,2,4-triazole, 3-[5-(4-chlorophenyl)-2,3-dimethyl-3-isoxazolidinyl]pyridine, (2-chloro-6-fluorophenyl)methyl 2-[1-[2-[3,5-bis(difluoromethyl)-1H-pyrazol-1-yl]acetyl]-4-piperidinyl]-4-thiazolecarboxylate, N′-[4-[[3-[(4-chlorophenyl)methyl]-1,2,4-thiadiazol-5-yl]oxy]-2,5-dimethylphenyl]-N-ethyl-N-methyl-methanimidamide, N-[2-[4- [[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, N′-[4-[4-chloro-3-(trifluoromethyl)phenoxy]-2,5-dimethylphenyl]-N-ethyl-N-methylmethanimidamide, N-cyclopropyl-3-(difluoromethyl)-5-fluoro-1-methyl-N-[[2-(1-methylethyl)phenyl]methyl]-1H-pyrazole-4-carboxamide, N-[[(cyclopropylmethoxy)amino][6-(difluoromethoxy)-2,3-difluoro-phenyl]methylene]benzeneacetamide, N-[2-(2,4-dichlorophenyl)-2-methoxy-1-methylethyl]-3-(difluoromethyl)-1-methyl-1H-pyrazole-4-carboxamide, N-(3′,4′-difluoro[1,1′-biphenyl]-2-yl)-3-(trifluoromethyl)-2-pyrazinecarboxamide, 3-(difluoromethyl)-N-(2,3-dihydro-1,1,3-trimethyl-1H-inden-4-yl)-1-methyl-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, 5,8-difluoro-N-[2-[3-methoxy-4-[[4-(trifluoromethyl)-2-pyridinyl]oxy]phenyl]ethyl]-4-quinazolinamine, 3-(difluoromethyl)-1-methyl-N-[2-(1,1,2,2-tetrafluoroethoxy)phenyl]-1H-pyrazole-4-carbox-amide, 1-[4-[4-[5R-[(2,6-difluorophenoxy)methyl]-4,5-dihydro-3-isoxazolyl]-2-thiazolyl]-1-piperdinyl]-2-[5-methyl-3-(trifluoromethyl)-1H-pyrazol-1-yl]ethanone, N-(1,1-dimethyl butyn-1-yl)-2-[(3-ethynyl-6-quinolinyl)oxy]-2-(methylthio)acetamide, 2,6-dimethyl-1H,5H-[1,4]dithiino[2,3-c:5,6-c′]dipyrrole-1,3,5,7(2H,6H)-tetrone, 2-[(3-ethynyl-6-quinolinyl)oxy]-N-[1-(hydroxymethyl)-1-methyl-2-propyn-1-yl]-2-(methylthio)acetamide, 4-fluorophenyl N-[1-[[[1-(4-cyanophenyl)ethyl]sulfonyl]methyl]propyl]carbamate, 5-fluoro-2-[(4-fluorophenyl)-methoxy]-4-pyrimidinamine, 5-fluoro-2-[(4-methylphenyl)methoxy]-4-pyrimidinamine, (3S,6S,7R,8R)-3- [[[4-methoxy-3-[[(2-methylpropoxy)carbonyl]oxy]-2-pyridinyl]carbonyl]-amino]-6-methyl-4,9-dioxo-8-(phenylmethyl)-1,5-dioxonan-7-yl-2-methylpropanoate, α-(methoxyimino)-N-methyl-2-[[[1-[3-(trifluoromethyl)phenyl]ethoxy]imino]methyl]benzene-acetamide, [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]carbonyl]-3-pyridinyl]oxy]methyl 2-methylpropanoate, pentyl N-[6- [[[[(1-methyl-1H-tetrazol-5-yl)phenylmethylene]amino]oxy]methyl]-2-pyridinyl]carbamate, pentyl N-[4- [[[[(1-methyl-1H-tetrazol-5-yl)phenylmethylene]amino]-oxy]methyl]-2-thiazolyl]carbamate, and pentyl N-[6- [[[[(Z)-(1-methyl-1H-tetrazol-5-yl)phenyl-methylene]amino]oxy]methyl]-2-pyridinyl]carbamate and (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate. Therefore of note is a fungicidal composition comprising as component (a) a compound of Formula 1 (or an N-oxide or salt thereof) and as component (b) at least one fungicide selected from the preceding list.

Of particular note are combinations of compounds of Formula 1 (or an N-oxide or salt thereof) (i.e. Component (a) in compositions) with azoxystrobin, benzovindiflupyr, bixafen, captan, carpropamid, chlorothalonil, copper hydroxide, copper oxychloride, copper sulfate, cymoxanil, cyproconazole, cyprodinil, diethofencarb, difenoconazole, dimethomorph, epoxiconazole, ethaboxam, fenarimol, fenhexamid, fluazinam, fludioxonil, fluindapyr, fluopyram, flusilazole, flutianil, flutriafol, fluxapyroxad, folpet, iprodione, isofetamid, isopyrazam, kresoxim-methyl, mancozeb, mandestrobin, meptyldinocap, metalaxyl (including metalaxyl-M/mefenoxam), mefentrifluconazole, metconazole, metrafenone, myclobutanil, oxathiapiprolin, penflufen, penthiopyrad, phosphorous acid (including salts thereof, e.g., fosetyl-aluminum), picoxystrobin, propiconazole, proquinazid, prothioconazole, pyraclostrobin, pyrimethanil, sedaxane spiroxamine, sulfur, tebuconazole, thiophanate-methyl, trifloxystrobin, zoxamide, α-(1-chlorocyclopropyl)-α-[2-(2,2-dichlorocyclopropyl)ethyl]-1H-1,2,4-triazole-1-ethanol, 2-[2-(1-chlorocyclopropyl)-4-(2,2-dichlorocyclopropyl)-2-hydroxybutyl]-1,2-dihydro-3H-1,2,4-triazole-3-thione, N-[2-(2,4-dichlorophenyl)-2-methoxy-1-methylethyl]-3-(difluoro-methyl)-1-methyl-1H-pyrazole-4-carboxamide, 3-(difluoromethyl)-N-(2,3-dihydro-1,1,3-tri-methyl-1H-inden-4-yl)-1-methyl-1H-pyrazole-4-carboxamide, 1-[4-[4-[5R-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, 1,1-dimethylethyl N-[6- [[[[(1-methyl-1H-tetrazol-5-yl)phenyl-methylene]amino]oxy]methyl]-2-pyridinyl]carbamate, 2,6-dimethyl-1H,5H-[1,4]dithiino[2,3-c:5,6-cl dipyrrole-1,3,5,7(2H,6H)-tetrone, 5-fluoro-2-[(4-fluorophenyl)methoxy]-4-pyrimidinamine, 5-fluoro-2-[(4-methylphenyl)methoxy]-4-pyrimidinamine, (αS)-[3-(4-chloro-2-fluorophenyl)-5-(2,4-difluorophenyl)-4-isoxazolyl]-3-pyridinemethanol, rel-1- [R²R,3S)-3-(2-chlorophenyl)-2-(2,4-difluorophenyl)-2-oxiranyl]methyl]-1H-1,2,4-triazole, rel-2- [[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole (i.e. as Component (b) in compostions).

Examples of other biologically active compounds or agents with which compounds of this invention can be formulated are: invertebrate pest control compounds or agents such as abamectin, acephate, acetamiprid, acrinathrin, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6- [[(1-cyclopropylethyl)amino]carbonyl]phenyl]-1-(3-chloro-2-pyridinyl)-1H-pyrazole-5-carboxamide), cycloxaprid ((5S,8R)-1-[(6-chloro-3-pyridinyl)methyl]-2,3,5,6,7,8-hexahydro-9-nitro-5,8-epoxy-1H-imidazo[1,2-a]azepine), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, flufenoxystrobin (methyl (αE)-2-[[2-chloro-4-(trifluoromethyl)phenoxy]methyl]-α-(methoxymethylene)benzeneacetate), fluensulfone (5-chloro-2-[(3,4,4-trifluoro-3-buten-1-yl)sulfonyl]thiazole), flupiprole (1-[2,6-dichloro-4-(trifluoromethyl)phenyl]-5-[(2-methyl-2-propen-1-yl)amino]-4-[(trifluoro-methyl)sulfinyl]-1H-pyrazole-3-carbonitrile), flupyradifurone (4-[[(6-chloro-3-pyridinyl)-methyl](2,2-difluoroethyl)amino]-2(5H)-furanone), tau-fluvalinate, flufenerim (UR-50701), flufenoxuron, fonophos, halofenozide, heptafluthrin ([2,3,5,6-tetrafluoro-4-(methoxymethyl)-phenyl]methyl 2,2-dimethyl-3-[(1Z)-3,3,3-trifluoro-1-propen-1-yl]cyclopropanecarboxylate), hexaflumuron, hydramethylnon, imidacloprid, indoxacarb, isofenphos, lufenuron, malathion, meperfluthrin ([2,3,5,6-tetrafluoro-4-(methoxymethyl)phenyl]methyl (1R,3S)-3-(2,2-dichloro-ethenyl)-2,2-dimethylcyclopropanecarboxylate), metaflumizone, metaldehyde, methamidophos, methidathion, methomyl, methoprene, methoxychlor, methoxyfenozide, metofluthrin, milbemycin oxime, momfluorothrin ([2,3,5,6-tetrafluoro-4-(methoxymethyl)phenyl]methyl-3-(2-cyano-1-propen-1-yl)-2,2-dimethylcyclopropanecarboxylate), monocrotophos, nicotine, nitenpyram, nithiazine, novaluron, noviflumuron (XDE-007), oxamyl, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriminostrobin (methyl (αE)-2- [ [[2-[(2,4-dichlorophenyl)amino]-6-(trifluoromethyl)-4-pyrimidinyl]oxy]methyl]-α-(methoxy-methylene)benzeneacetate), pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Also in certain instances, combinations of a compound of the invention with other biologically active compounds or agents can result in a less-than-additive (i.e. safening) effect on organisms beneficial to the agronomic environment. For example, a compound of the invention may safen a herbicide on crop plants or protect a beneficial insect species (e.g., insect predators, pollinators such as bees) from an insecticide.

Fungicides of note for formulation with compounds of Formula 1 to provide mixtures useful in seed treatment include but are not limited to amisulbrom, azoxystrobin, boscalid, carbendazim, carboxin, cymoxanil, cyproconazole, difenoconazole, dimethomorph, florylpicoxamid, fluazinam, fludioxonil, flufenoxystrobin, fluquinconazole, fluopicolide, fluoxastrobin, flutriafol, fluxapyroxad, ipconazole, iprodione, metalaxyl, mefenoxam, mefentrifluconazole, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Invertebrate pest control compounds or agents with which compounds of Formula 1 can be formulated to provide mixtures useful in seed treatment include but are not limited to abamectin, acetamiprid, acrinathrin, afidopyropen, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyclaniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, fluensulfone, flufenoxuron, flufiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, momfluorothrin, nitenpyram, nithiazine, novaluron, oxamyl, pyflubumide, pymetrozine, pyrethrin, pyridaben, pyriminostrobin, pyridalyl, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumuron, Bacillus thuringiensis delta-endotoxins, strains of Bacillus thuringiensis and strains of Nucleo polyhydrosis viruses.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM I-1582 (GB-126) which is commercially available as BioNem™. A suitable Bacillus cereus strain is strain NCMM 1-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inoculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inoculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The following TESTS demonstrate the control efficacy of compounds of this invention on specific pathogens. The pathogen control protection afforded by the compounds is not limited, however, to these species. See Index Tables A and B below for compound descriptions. The abbreviation “Cmpd.” stands for “Compound”, and the abbreviation “Ex.” stands for “Example” and is followed by a number indicating in which example the compound is prepared. The numerical value reported in the column “MS” is the molecular weight of the highest isotopic abundance positively charged parent ion (M+1) formed by addition of H+(molecular weight of 1) to the molecule having the highest isotopic abundance, or the highest isotopic abundance negatively charged ion (M−1) formed by loss of H+(molecular weight of 1). The presence of molecular ions containing one or more higher atomic weight isotopes of lower abundance (e.g., 37Cl, 81Br) is not reported. The reported MS peaks were observed by mass spectrometry using electrospray ionization (ESI) or atmospheric pressure chemical ionization (APCI).

INDEX TABLE A

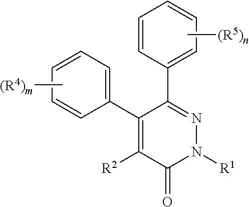

| | | | | | MS | |
|---|---|---|---|---|---|---|
| Cmpd. No. | R¹ | R² | (R⁴)ₘ | (R⁵)ₙ | (M + 1) | m.p. (° C.) |
| 1 | Et | Cl | 2,6-di-F | 3,5-di-MeO | | 149-152 |
| 2 | Et | H | 2,6-di-F | 2,6-di-Cl, 3,5-di-MeO | | 205-208 |
| 3 | Et | H | 3,5-di-MeO | 3,5-di-MeO | 397 | |
| 4 (Ex. 10) | Et | H | 3,5-di-MeO | 2,6-di-F | 373 | |
| 5 (Ex. 9) | CH₃ | H | 2,6-di-F | 2,6-di-F | 335 | |
| 6 (Ex. 14) | CH₃ | CN | 2-Cl, 4-F | 2-Cl, 4-F | | 137-140 |
| 7 | Et | CN | 2-Cl, 4-F | 2-Cl, 4-F | | 79-82 |
| 8 | Et | H | 2,6-di-F | 2-Cl, 3,5-di-MeO | | 137-140 |
| 9 | Et | Cl | 2,6-di-F | 2-Cl, 3,5-di-MeO | | 170-173 |
| 10 (Ex. 11) | Et | Cl | 3,5-di-MeO | 2,6-di-F | 408 | |
| 11 | Et | H | 2-Cl, 4-F | 2-Cl, 4-F | 381 | |
| 12 (Ex. 7) | CH₃ | H | 2,6-di-F | 2-Cl, 3,5-di-MeO | | 172-176 |
| 13 (Ex. 5) | CH₃ | H | 2,6-di-F | 2-NO₂, 3,5-di-MeO | | 174-178 |
| 14 (Ex. 7) | CH₃ | H | 2,6-di-F | 3,5-di-MeO, 4-Cl | | 221-225 |
| 15 | Et | Br | 2,6-di-F | 3,5-di-MeO | | 143-146 |
| 16 | Et | Br | 2,6-di-F | 2-Br, 3,5-di-MeO | | 167-170 |
| 17 | Et | H | 2,6-di-F | 2-Br, 3,5-di-MeO | | 113-116 |
| 18 | CH₃ | H | 2-Cl, 4-F | 3,5-di-MeO | | 163-166 |
| 21 (Ex. 12) | CH₃ | CH₃ | 2,6-di-F | 3,5-di-MeO | 373 | |
| 22 | Et | CH₃ | 2,6-di-F | 2-Cl, 3,5-di-MeO | 421 | |
| 23 | Et | H | 2,6-di-F | 2,6-di-F | 349 | |
| 24 | Et | Cl | 2-Cl, 4-F | 2-Cl, 4-F | 416 | |
| 25 | CH₃ | H | 2-Cl, 4-F | 2-Cl, 3,5-di-MeO | | 121-125 |
| 26 | Et | H | 2-Cl, 4-F | 3,5-di-MeO | | 152-156 |
| 27 | CH₃ | H | 2,6-di-F | 3-F, 5-MeO | 347 | |
| 28 | CH₃ | H | 2,6-di-F | 2-Cl, 5-F | 351 | |
| 29 | CH₃ | H | 2,6-di-F | 2-Cl, 3-F | 351 | |
| 30 | CH₃ | H | 2,6-di-F | 2-Cl, 3-MeO | 363 | |
| 31 | CH₃ | H | 2,6-di-F | 2-Cl, 3,5-di-MeO, 6-NO₂ | | 249-253 |
| 32 | Et | H | 2,6-di-F | 3,5-di-MeO | | 103-106 |
| 34 (Ex. 8) | CH₃ | Br | 2,6-di-F | 3,5-di-MeO | | 153-157 |
| 35 (Ex. 6) | CH₃ | Cl | 2,6-di-F | 2-Cl, 3,5-di-MeO | | 192-196 |
| 36 (Ex. 3) | CH₃ | Cl | 2,6-di-F | 3,5-di-MeO | | 150-154 |
| 37 (Ex. 4) | CH₃ | Cl | 2,6-di-F | 2-NO₂, 3,5-di-MeO | | 223-227 |
| 38 (Ex. 2) | CH₃ | H | 2,6-di-F | 3,5-di-MeO | | 132-136 |
| 40 | CH₃ | Et | 2,4-di-F | 2-Cl, 3,5-di-MeO | 421 | |
| 41 | CH₃ | Et | 2,4-di-F | 2,6-di-Cl, 3,5-di-MeO | 457 | |
| 42 | CH₃ | H | 2-Cl, 4-F | 2-Cl, MeNHC(═O)CH₂O | | 111-114 |
| 43 | CH₃ | H | 2-Cl, 4-F | 2-Cl, 5-c-PrCH₂O | | 129-132 |
| 44 | CH₃ | CH₃ | 2-Cl, 4-F | 2-Cl, 4-F | 381 | 126-129 |
| 45 | CH₃ | Et | 2-Cl, 4-F | 2-Br, 6-Cl, 3,5-di-MeO | 517 | 155-158 |
| 46 | CH₃ | Et | 2,6-di-F | 2,6-di-F | 363 | |
| 47 | CH₃ | CH₃ | 2-Cl, 4-F | 2,6-di-Cl, 3,5-di-MeO | | 217-220 |
| 48 | CH₃ | MeO | 2,4-di-F | 2-Cl, 5-MeO | 393 | 126-127 |
| 49 | CH₃ | Cl | 2-Cl, 4-F | 2-Cl, 5-C≡N | 408 | |
| 50 | CH₃ | MeO | 2,6-di-F | 2-Cl, 5-MeO | 393 | 167-168 |
| 51 | CH₃ | Cl | 2-Cl, 4-F | 2-Cl, 3-C≡N, 5-MeO | 438 | 154-156 |
| 52 | CH₃ | Cl | 2-Cl, 4-F | 3-C≡N, 4-Cl, 5-MeO | 438 | 160-162 |
| 53 | CH₃ | MeO | 2-Cl, 4-F | 2-Cl, 5-C≡N | 404 | 124-126 |
| 54 | CH₃ | H | 2,6-di-F | 2-Br, 3-F, 5-MeO | 427 | |
| 55 | CH₃ | Cl | 2-Cl, 4-F | 3,5-di-MeO | 409 | 154-156 |
| 56 | CH₃ | H | 2,6-di-F, 4-MeO | 3,5-di-MeO | 389 | |
| 57 | CH₃ | Cl | 2,6-di-F | 2-Cl, 5-MeO | | 134-137 |
| 58 | CH₃ | Cl | 2-Cl, 4-F | 2-Cl, 3,5-MeO | 443 | 85-90 |
| 59 | CH₃ | Cl | 2-Cl, 4-F | 2,6-di-Cl, 3,5-di-MeO | 447 | 174-176 |
| 60 | CH₃ | Cl | 2-Cl, 4-F | 2-Cl, 4-F | 401 | |
| 61 | CH₃ | Br | 2-Cl, 4-F | 2-Br, 3,5-di-MeO | | 167-170 |
| 62 | CH₃ | H | 2,4-di-F | 3,5-di-MeO | 359 | |
| 63 | CH₃ | CH₃ | 2-Cl, 4-F | 3,5-di-MeO, 4-Br | | |
| 64 | CH₃ | CH₃ | 2-Cl, 4-F | 2-Br, 3,5-di-MeO | 467 | |
| 65 | CH₃ | CH₃ | 2-Cl, 4-F | 3,5-di-MeO | | 117-120 |
| 66 | CH₃ | CH₃ | 2-Cl, 4-F | 2-Cl, 3,5-diMeO | | 140-143 |
| 67 | CH₃ | Br | 2-Cl, 4-F | 3,5-di-MeO | 453 | |
| 68 | CH₃ | CH₃ | 2,6-di-F | 2-Cl, 5-MeO | | 135-138 |
| 69 | CH₃ | Br | 2,6-di-F | 2-Cl, 5-MeO | | 164-167 |
| 70 | CH₃ | H | 2-Cl, 4-F | 2-Cl, 4-F | 367 | |
| 71 | CH₃ | H | 2,6-di-F | 3-C≡N, 5-MeO | 354 | |
| 72 | CH₃ | Cl | 2-Cl, 4-F | 2-F | 367 | |
| 73 | CH₃ | Cl | 2-Cl, 4-F | 2-Cl | 383 | |
| 74 | Et | MeO | 2,6-di-F | 2-Br, 3,5-di-MeO | 481 | |
| 75 | Et | MeO | 2,6-di-F | 2-Cl, 3,5-MeO | 437 | |
| 76 | CH₃ | Cl | 2-Cl, 4-F | 3-F, 5-MeO | 397 | 127-129 |
| 77 | CH₃ | Cl | 2-Cl, 4-F | 2-Br, 3,5-di-MeO | 487 | |
| 78 | CH₃ | MeO | 2-Cl, 4-F | 3,5-di-MeO | 405 | |
| 79 | CH₃ | MeO | 2-Cl, 4-F | 2-Br, 3,5-di-MeO | 483 | |
| 80 | CH₃ | MeO | 2-Cl, 4-F | 2-Cl, 3,5-MeO | 439 | 130-133 |
| 81 | CH₃ | MeO | 2-Cl, 4-F | 2,6-di-Cl, 3,5-di-MeO | 732 | |
| 82 | CH₃ | Cl | 2-Cl, 4-F | 2-Cl, 5-F | 401 | 136-138 |
| 83 | CH₃ | Cl | 2-Cl, 4-F | 2-Cl, 5-MeO | 413 | 165-168 |
| 84 | CH₃ | H | 2,6-di-F | 2-Cl, 4-F | 351 | |
| 85 | CH₃ | H | 2,6-di-F | 2-Cl, 5-MeO | 363 | 161-164 |
| 86 | CH₃ | H | 2-Cl, 4-F | 2-Cl, 5-MeOC(═O)CH₂O | | 150-153 |
| 87 | CH₃ | H | 2-Cl, 4-F | 2-Cl, 5-EtO | | 123-126 |
| 88 | CH₃ | H | 2-Cl, 4-F | 2-Cl, N≡CCH₂O | | 137-140 |
| 89 | CH₃ | H | 2-Cl, 4-F | 2-Cl, 5-n-PrO | | 86-89 |
| 90 | CH₃ | H | 2-Cl, 4-F | 2-Cl, 5-CH₂═CHCH₂O | | 93-96 |
| 91 | CH₃ | H | 2-Cl, 4-F | 2-Cl, 5-CH≡CCH₂O | | 102-105 |
| 92 | CH₃ | H | 2-Cl, 4-F | 2-Cl, 5-OH | | 197-200 |
| 93 | CH₃ | H | 2-Cl, 4-F | 2-Cl, 4-OHC(═O)CH₂O | | 200-205 |
| 94 | CH₃ | MeO | 2-Cl, 4-F | 2-Cl, 3-MeO, 5-C≡N | 484 | 143-144 |
| 95 | CH₃ | Cl | — | 2-Cl, 5-MeO | 361 | |
| 96 | CH₃ | Cl | 2,6-di-F, 4-MeO | 2-Cl, 5-MeO | 427 | |
| 97 | CH₃ | Cl | 2-Br, 4-F | 2-Cl, 5-MeO | 459 | 152-154 |
| 98 | CH₃ | CH₃ | 2-Cl, 4-F | 2-Cl, 5-i-BuO | 436 | |
| 99 | CH₃ | CH₃ | 2-Cl, 4-F | 2-Cl, 5-n-PrO | 422 | |
| 100 | Et | C≡N | 2,6-di-F | 2-Br, 3,5-di-MeO | 476 | |
| 101 | CH₃ | MeO | 2-Cl, 4-F | 2-Cl, 5-MeO | 409 | |
| 102 | CH₃ | CH₃ | 2-Cl, 4-F | 2-Cl, 5-MeO | 394 | 149-150 |
| 103 | CH₃ | MeO | 2-Br, 4-F | 2-Cl, 5-MeO | 455 | |
| 104 | CH₃ | Cl | 2-Cl, 4-F | 2,3-di-Cl | 419 | 151-153 |
| 105 | CH₃ | Cl | 2-Cl, 4-F | 2-F, 3-Cl | 401 | 152-154 |
| 106 | CH₃ | Cl | 2-Cl, 4-F | 2-Br, 3-F | 447 | 147-149 |
| 107 | CH₃ | Cl | 2-Cl, 4-F | 2,3-di-F | 385 | 146-148 |
| 108 | CH₃ | MeO | 2-Cl, 4-F | 2,3-di-Cl | 413 | 134-135 |
| 109 | CH₃ | MeO | 2-Cl, 4-F | 2-F, 3-Cl | 397 | 118-119 |
| 110 | CH₃ | MeO | 2-Cl, 4-F | 2-Br, 3-F | 441 | 126-128 |
| 111 | CH₃ | CH₃ | 2-Cl, 4-F | 2-Cl, 3,5-di-OH | 397 | 143-145 |
| 112 | CH₃ | CH₃ | 2-Cl, 4-F | 2-Cl, 3,5-di-EtO | 452 | 156-158 |
| 113 | CH₃ | Et | 2-Cl, 4-F | 2-MeO, 5-Br | 453 | 155-157 |
| 114 | CH₃ | Et | 2-Cl, 4-F | 2-Cl, 5-EtO | 422 | |
| 115 | CH₃ | CH₃ | 2-Cl, 4-F | 2-MeO | 359 | 135-136 |
| 116 | CH₃ | MeO | 2-Cl, 4-F | 2,3-di-F | 381 | 105-106 |
| 117 | CH₃ | H | 2-Cl, 4-F | 2-Cl, 5-MeO | 379 | 131-134 |
| 118 | CH₃ | Cl | 2-Cl, 4-F | 2-Br, 5-MeO | 459 | |
| 119 | CH₃ | Cl | 2-Cl, 4-F | 2-Br, 5-F | 447 | 129-131 |
| 120 | CH₃ | MeO | 2-Cl, 4-F | 2-Cl, 5-F | 397 | |
| 121 | CH₃ | MeO | 2-Cl, 4-F | 2-Br, 5-F | 443 | |
| 122 | CH₃ | MeOCH₂ | 2-Cl, 4-F | 2-Cl, 4-F | 411 | |
| 123 | CH₃ | Me₂C(OH) | 2-Cl, 4-F | 2-Cl, 4-F | | 138-142 |
| 124 | CH₃ | CH₃C(═O) | 2-Cl, 4-F | 2-Cl, 4-F | 409 | |
| 125 | CH₃ | n-Pr | 2-Cl, 4-F | 2-Cl, 5-MeO | 421 | |
| 126 | CH₃ | H | 2-Cl, 4-F | 2-Cl, 5-F | 367 | 56-57 |
| 127 | CH₃ | CH₃ | 2-Cl, 4-F | 2-Cl, 5-i-PrO | 422 | 100-102 |
| 128 | CH₃ | CH₃ | 2-Cl, 4-F | 2-Cl, 5-F | 381 | 50-52 |
| 129 | CH₃ | Et | 2-Cl, 4-F | 2-Cl, 5-F | 395 | |
| 130 | CH₃ | F₂CH | 2-Cl, 4-F | 2-Cl, 4-F | 417 | |
| 131 | CH₃ | MeCH(OH) | 2-Cl, 4-F | 2-Cl, 4-F | | 138-141 |
| 132 | CH₃ | CH₃CFH | 2-Cl, 4-F | 2-Cl, 4-F | 413 | |
| 133 | CH₃ | NH₂C(═O) | 2-Cl, 4-F | 2-Cl, 4-F | 410 | |
| 134 | CH₃ | Et | 2-Cl, 4-F | 2-Cl, 5-MeO | 408 | |
| 135 | CH₃ | CH₃ | 2-Cl, 4-F | 2-Me, 5-MeO | 374 | |
| 136 (Ex. 15) | CH₃ | CH₃ | 2-Cl, 4-F | 2-Cl, 5-Me | 377 | |
| 137 | CH₃ | CH₃ | 2-Cl, 4-F | 2,3-di-F, 5-MeO | 396 | |
| 138 | CH₃ | CH₃ | 2-Cl, 4-F | 2-F, 5-MeO | 378 | |
| 139 | CH₃ | c-Pr | 2-Cl, 4-F | 2-Cl, 5-MeO | 420 | |
| 140 | CH₃ | Cl | 2-Cl, 4-F | 2-Cl, 3-MeO | 415 | 163-164 |
| 141 (Ex. 16) | CH₃ | Cl | 2-Cl, 4-F | — | 349 | 142-144 |
| 142 (Ex. 17) | CH₃ | MeO | 2-Cl, 4-F | — | 345 | 124-126 |
| 143 | CH₃ | EtOC(═O) | 2-Cl, 4-F | 2-Cl, 4-F | | 146-149 |
| 144 | CH₃ | OHC(═O) | 2-Cl, 4-F | 2-Cl, 4-F | | 136-140 |
| 145 | CH₃ | Cl | 2-Cl, 4-F | 2-Cl, 3-F | 403 | 148-150 |
| 146 | CH₃ | MeO | 2-Cl, 4-F | 2-Cl, 3-MeO | 409 | 141-143 |
| 147 | CH₃ | Cl | 2,4-di-F | 2-Br, 3,5-di-MeO | 473 | |
| 148 | CH₃ | Cl | 2,4-di-F | 2-Cl, 3,5-di-MeO | 429 | |
| 149 | CH₃ | Et | 2,4-di-F | 2-Br, 3,5-di-MeO | 467 | |
| 150 | CH₃ | Et | 2,4-di-F | 2,6-di-Br, 3,5-di-MeO | 545 | |
| 151 | CH₃ | MeO | 2,4-di-F | 2-Br, 3,5-di-MeO | 469 | 112-114 |
| 152 | CH₃ | MeO | 2,4-di-F | 2-Cl, 3,5-di-MeO | 423 | |
| 153 | CH₃ | EtOC(═O) | 2-Cl, 4-F | 2-Cl, 4-F | 441 | |
| 154 | CH₃ | MeO | 2-Cl, 4-F | 2-Cl, 3-F | 397 | 120-122 |
| 155 | CH₃ | Cl | 2-Cl, 4-F | 2-F, 3,5-di-MeO | 427 | |
| 156 | CH₃ | Cl | 2-Cl, 4-F | 2-Br, 4-F | 447 | 136-138 |
| 157 | CH₃ | Cl | 2-Cl, 4-F | 2-Cl, 3-F, 5-MeO | 431 | 113-115 |
| 158 | CH₃ | Cl | 2-Cl, 4-F | 2-Br, 3-MeO, 5-F | 477 | 152-153 |
| 159 | CH₃ | Cl | 2-Cl, 4-F | 2-Br, 3-F, 5-MeO | 477 | 143-144 |
| 160 | CH₃ | MeO | 2-Cl, 4-F | 2-Cl | 379 | |
| 161 | CH₃ | CH₃ | 2-Cl, 4-F | 2-Cl, 5-OH | 379 | |
| 162 | CH₃ | MeO | 2-Cl, 4-F | 2-Br, 4-F | 443 | |
| 163 | CH₃ | Cl | 2-Cl, 4-F | 2-F, 4-MeO | 397 | |
| 164 | CH₃ | MeO | 2-Cl, 4-F | 2-Cl, 3-F, 5-MeO | 427 | |
| 165 | CH₃ | MeO | 2-Cl, 4-F | 2-Br, 3-F, 5-MeO | 473 | |
| 166 | CH₃ | MeO | 2-Cl, 4-F | 2-F, 4-MeO | 393 | |
| 167 | CH₃ | Et | 2-Cl, 4-F | 2-Cl, 4-F | 397 | |
| 168 | CH₃ | OHCH₂ | 2-Cl, 4-F | 2-Cl, 4-F | 397 | |
| 169 | CH₃ | CH₃ | 2-Cl, 4-F | 2,6-di-Cl, 3,5-di-MeO | | 212-215 |
| 170 | CH₃ | ClCH₂ | 2-Cl, 4-F | 2-Cl, 4-F | 415 | |
| 171 | CH₃ | FCH₂ | 2-Cl, 4-F | 2-Cl, 4-F | 399 | |
| 172 | CH₃ | OHCH₂ | 2-Cl, 4-F | 2-Cl, 4-F | 399 | |
| 173 | CH₃ | Cl | 2-Cl, 4-F | 3-C≡N, 5-MeO | 404 | 74-76 |
| 174 | CH₃ | MeO | 2-Cl, 4-F | 3-C≡N, 5-MeO | 400 | 187-188 |
| 175 | CH₃ | CH₃ | 2-Cl, 4-F | 2-Cl, 5-EtO | 407 | |
| 176 | CH₃ | MeON(Me) | 2-Cl, 4-F | 2-Cl, 4-F | | 100-103 |
| | | C(═O) | | | | |
| 177 | CH₃ | N≡CCH₂ | 2-Cl, 4-F | 2-Cl, 4-F | | 127-130 |
| 178 | CH₃ | Et | 2,6-di-F | 2-Cl, 3-MeO | 391 | 146-147 |
| 179 | CH₃ | Et | 2,6-di-F | 2-Cl, 5-MeO | 391 | |
| 180 | CH₃ | Et | 2,6-di-F | 2-Cl, 3-F, 5-MeO | 409 | |
| 181 | CH₃ | Et | 2,6-di-F | 2-Br, 3-F, 5-MeO | 453 | |
| 182 | CH₃ | OH | 2-Cl, 4-F | 2-Cl | 365 | |
| 183 | CH₃ | Cl | 2-F | 2-Cl, 5-MeO | 379 | 134-136 |
| 184 | CH₃ | Cl | 2-Cl | 2-Cl, 5-MeO | 397 | 145-147 |
| 185 | CH₃ | Cl | 2,4-di-F | 2-Cl, 5-MeO | 397 | 195-196 |
| 186 | CH₃ | Cl | 2-Cl, 4-N≡C | 2-Cl, 5-MeO | 420 | 160-162 |
| 187 | CH₃ | CH₃ | 2,4-di-F | 2-Cl, 3,5-di-MeO | 407 | 121-123 |
| 188 | CH₃ | CH₃ | 2,4-di-F | 2,6-di-Cl, 3,5-MeO | 443 | 111-113 |
| 189 | CH₃ | CH₃ | 2,4-di-F | 2-Br, 3,5-di-MeO | 453 | 114-116 |
| 190 | CH₃ | CH₃ | 2,4-di-F | 2,6-di-Br, 3,5-di-MeO | 531 | 180-182 |
| 191 | CH₃ | H | 2,6-di-F | — | 299 | |
| 192 | CH₃ | H | 2,6-di-F | 2-F, 4-MeO | 347 | |
| 193 | CH₃ | Cl | 2,6-di-F | — | 333 | |
| 194 | CH₃ | CH₃ | 2,6-di-F | 2-Cl, 3,5-di-MeO | 407 | |
| 195 | CH₃ | H | 2,6-di-F | 2-F | 317 | |
| 196 | CH₃ | H | 2,6-di-F | 2-Cl | 333 | |
| 199 | CH₃ | H | 2,6-di-F | 3,5-di-F | 335 | |
| 200 | Et | H | 2,6-di-F | 2-Me, 3,5-di-MeO | 387 | |
| 202 | Et | H | 2-Cl, 4-F | 2-Cl, 3,5-MeO | 423 | |
| 203 | CH₃ | H | 2-Cl, 4-F | 3,5-di-MeO, 4-Cl | 409 | |
| 204 | CH₃ | H | 3-N≡C, 5-MeO | 3-N≡C, 5-MeO | 373 | |
| 205 | CH₃ | H | 2,6-di-F | 2-Cl, 3-F, 5-MeO | 381 | |
| 206 | CH₃ | H | 2,6-di-F | 2-Cl, 3-MeO, 5-F | 381 | |

INDEX TABLE B

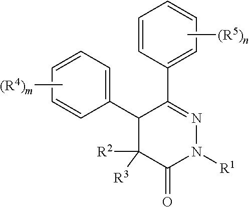

| Cmpd. No. | R¹ | R² | R³ | R⁴ | R⁵ | MS | m.p. (° C.) |
|---|---|---|---|---|---|---|---|
| 19 | CH₂CH₃ | H | H | 2-Cl, 4-F | 3,5-di-MeO | | 165-168 |
| 20 | CH₃ | H | H | 2-Cl, 4-F | 3,5-di-MeO | | 122-125 |
| 33 | CH₂CH₃ | H | H | 2,6-di-F | 3,5-di-MeO | | 119-122 |
| (Ex. 13) | | | | | | | |
| 39 | CH₃ | H | H | 2,6-di-F | 3,5-di-MeO | | 139-142 |
| (Ex. 1) | | | | | | | |
| 197 | CH₃ | H | H | 2,6-di-F | 2-Cl, 3,5-di-MeO | 395 | |
| | | | | | | (M + 1) | |
| 198 | CH₃ | CH₃ | H | 2-Cl, 4-F | 3,5-di-MeO | | 130-135 |
| 201 | Et | CH₃ | H | 2-Cl, 4-F | 3,5-di-MeO | | 116-120 |
| 207 | CH₃ | H | H | 2,6-di-F | 2-Cl, 5-MeO | | 117-121 |
| 208 | CH₃ | CH₃ | H | 2,6-di-F | 2-Cl, 5-MeO | | 144-147 |

### BIOLOGICAL EXAMPLES OF THE INVENTION

General protocol for preparing test suspensions for Tests A-F: the test compounds were first dissolved in acetone in an amount equal to 3% of the final volume and then suspended at the desired concentration (in ppm) in acetone and purified water (50/50 mix by volume) containing 250 ppm of the surfactant PEG400 (polyhydric alcohol esters). The resulting test suspensions were then used in Tests A-F.

### Test A

The test solution was sprayed to the point of run-off on wheat seedlings. The following day the seedlings were inoculated with a spore suspension of Zymoseptoria tritici (the causal agent of wheat leaf blotch) and incubated in a saturated atmosphere at 24° C. for 48 h, and then moved to a growth chamber at 20° C. for 17 days, after which time disease ratings were made.

### Test B

The test solution was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 7 days, after which time disease ratings were made.

### Test C

The test suspension was sprayed to the point of run-off on wheat seedlings. The following day the seedlings were inoculated with a spore dust of Blumeria graminis f. sp. tritici, (also known as Erysiphe graminis f. sp. tritici, the causal agent of wheat powdery mildew) and incubated in a growth chamber at 20° C. for 8 days, after which time visual disease ratings were made.

### Test D

The test solution was sprayed to the point of run-off on soybean seedlings. The following day the seedlings were inoculated with a spore suspension of Phakopsora pachyrhizi (the causal agent of Asian soybean rust) and incubated in a saturated atmosphere at 22° C. for 24 h and then moved to a growth chamber at 22° C. for 8 days, after which time visual disease ratings were made.

### Test E

The test suspension was sprayed to the point of run-off on tomato seedlings. The following day the seedlings were inoculated with a spore suspension of Botrytis cinerea (the causal agent of tomato Botrytis) and incubated in a saturated atmosphere at 20° C. for 48 h, and then moved to a growth chamber at 24° C. for 3 days, after which time visual disease ratings were made.

### Test F

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 3 days, after which time visual disease ratings were made.

Results for Tests A-F are given in Table A below. A rating of 100 indicates 100% disease control and a rating of 0 indicates no disease control (relative to the controls). A dash (—) indicates the compound was not tested.

TABLE A

| Cmpd | Rate | Test | Test | Test | Test | Test | Test |
|---|---|---|---|---|---|---|---|
| No. | in ppm | A | B | C | D | E | F |
| 1 | 250 | 87 | 55 | 0 | 0 | 90 | 0 |
| 2 | 250 | 2 | 0 | 0 | 0 | 0 | 0 |
| 3 | 250 | 7 | 0 | 0 | 0 | 0 | 0 |
| 4 | 250 | 66 | 0 | 0 | 0 | 9 | 0 |
| 5 | 250 | 42 | 0 | 100 | 0 | 99 | 0 |
| 6 | 250 | 100 | 100 | 92 | 0 | 99 | 99 |
| 7 | 250 | 98 | 74 | 0 | 0 | 99 | 0 |
| 8 | 50 | 100 | 96 | 97 | 75 | 99 | 0 |
| 9 | 50 | 100 | 100 | 99 | 99 | 99 | 91 |
| 10 | 250 | 0 | 0 | 0 | 0 | 0 | 0 |
| 11 | 250 | 2 | 9 | 64 | 0 | 87 | 0 |
| 12 | 250 | 100 | 100 | 100 | 100 | 99 | 0 |
| 13 | 250 | 94 | 99 | 81 | 63 | 99 | 9 |
| 14 | 250 | 88 | 80 | 0 | 0 | 0 | 0 |
| 15 | 50 | 78 | 41 | 13 | 0 | 68 | 0 |
| 16 | 50 | 99 | 97 | 97 | 84 | 99 | 94 |
| 17 | 50 | 98 | 99 | 94 | 73 | 99 | 0 |
| 18 | 250 | 99 | 98 | 96 | 0 | 99 | 0 |
| 19 | 250 | 0 | 0 | 0 | 0 | 0 | 0 |
| 20 | 250 | 51 | 9 | 21 | 0 | 31 | 0 |
| 21 | 250 | 100 | 100 | 100 | 100 | 100 | 99 |
| 22 | 50 | 100 | 100 | 98 | 98 | 100 | 79 |
| 23 | 250 | 0 | 0 | 0 | 0 | 0 | 0 |
| 24 | 250 | 100 | 80 | 93 | 0 | 100 | 0 |
| 25 | 50 | 100 | 100 | 98 | 94 | 100 | 85 |
| 26 | 250 | 12 | 55 | 96 | 0 | 99 | 0 |
| 27 | 250 | 99 | 99 | 97 | 56 | 96 | 0 |
| 28 | 250 | 100 | 100 | 100 | 0 | 100 | 0 |
| 29 | 250 | 100 | 98 | 99 | 25 | 100 | 0 |
| 30 | 250 | 100 | 99 | 84 | 98 | 100 | 41 |
| 31 | 50 | 0 | 9 | 0 | 0 | 0 | 0 |
| 32 | 250 | 0 | 9 | 29 | 0 | 80 | 0 |
| 33 | 250 | 0 | 55 | 0 | 0 | 0 | 0 |
| 34 | 250 | 100 | 89 | 95 | 99 | 99 | 99 |
| 35 | 250 | 100 | 100 | 100 | 100 | 99 | 99 |
| 36 | 250 | 100 | 100 | 99 | 93 | 100 | 99 |
| 37 | 250 | 99 | 100 | 98 | 100 | 99 | 99 |
| 38 | 250 | 98 | 99 | 81 | 0 | 98 | 0 |
| 39 | 250 | 98 | 86 | 69 | 0 | 99 | 0 |
| 40 | | — | — | — | — | — | — |
| 41 | | — | — | — | — | — | — |
| 42 | | — | — | — | — | — | — |
| 43 | | — | — | — | — | — | — |
| 44 | 50 | 100 | 100 | 100 | 100 | 93 | 100 |
| 45 | 50 | 0 | 0 | 69 | 0 | 0 | 0 |
| 46 | 50 | 99 | 98 | 95 | 0 | 88 | 99 |
| 47 | 50 | 52 | 68 | 43 | 0 | 91 | 0 |
| 48 | 50 | 100 | 100 | 99 | 97 | 98 | 99 |
| 49 | 50 | 100 | 100 | 98 | 96 | 96 | 99 |
| 50 | 50 | 100 | 98 | 99 | 36 | 96 | 100 |
| 51 | 50 | 100 | 100 | 99 | 100 | 96 | 100 |
| 52 | 50 | 100 | 100 | 99 | 98 | 92 | 100 |
| 53 | 50 | 100 | 100 | 99 | 68 | 92 | 40 |
| 54 | | — | — | — | — | — | — |
| 55 | 50 | 99 | 100 | 98 | 99 | 99 | 99 |
| 56 | | — | — | — | — | — | — |
| 57 | 50 | 100 | 100 | 99 | 99 | 99 | 100 |
| 58 | 50 | 100 | 100 | 100 | 100 | 99 | 100 |
| 59 | 50 | 4 | 74 | 0 | 0 | 0 | 0 |
| 60 | 50 | 100 | 100 | 99 | 93 | 98 | 99 |
| 61 | 50 | 100 | 100 | 94 | 99 | 100 | 99 |
| 62 | | — | — | — | — | — | — |
| 63 | | — | — | — | — | — | — |
| 64 | 50 | 100 | 100 | 100 | 100 | 100 | 100 |
| 65 | 50 | 99 | 100 | 98 | 94 | 100 | 99 |
| 66 | 50 | 100 | 100 | 100 | 100 | 100 | 100 |
| 67 | 50 | 99 | 100 | 95 | 98 | 99 | 99 |
| 68 | 50 | 100 | 100 | 100 | 100 | 99 | 100 |
| 69 | 50 | 100 | 99 | 100 | 100 | 100 | 100 |
| 70 | 50 | 93 | 41 | 76 | 52 | 98 | 0 |
| 71 | 50 | 8 | 0 | 0 | 0 | 16 | 0 |
| 72 | 50 | 100 | 99 | 99 | 75 | 98 | 99 |
| 73 | 50 | 100 | 100 | 99 | 97 | 99 | 99 |
| 74 | 50 | 100 | 99 | 98 | 75 | 99 | 0 |
| 75 | 50 | 100 | 99 | 98 | 69 | 97 | 0 |
| 76 | 50 | 100 | 100 | 99 | 99 | 97 | 99 |
| 77 | 50 | 100 | 100 | 99 | 100 | 97 | 100 |
| 78 | 50 | 100 | 99 | 98 | 59 | 95 | 99 |
| 79 | 50 | 100 | 100 | 100 | 100 | 97 | 100 |
| 80 | 50 | 100 | 100 | 100 | 100 | 99 | 100 |
| 81 | 50 | 0 | 41 | 0 | 0 | 0 | 0 |
| 82 | 50 | 100 | 100 | 100 | 100 | 64 | 100 |
| 83 | 50 | 100 | 100 | 99 | 100 | 92 | 100 |
| 84 | | — | — | — | — | — | — |
| 85 | | — | — | — | — | — | — |
| 86 | | — | — | — | — | — | — |
| 87 | | — | — | — | — | — | — |
| 88 | | — | — | — | — | — | — |
| 89 | | — | — | — | — | — | — |
| 90 | | — | — | — | — | — | — |
| 91 | | — | — | — | — | — | — |
| 92 | | — | — | — | — | — | — |
| 93 | | — | — | — | — | — | — |
| 94 | 50 | 100 | 100 | 100 | 96 | 96 | 100 |
| 95 | 50 | 100 | 99 | 97 | 0 | 96 | 24 |
| 96 | 50 | 100 | 100 | 97 | 100 | 96 | 100 |
| 97 | 50 | 100 | 100 | 100 | 100 | 96 | 99 |
| 98 | | — | — | — | — | — | — |
| 99 | | — | — | — | — | — | — |
| 100 | 50 | 100 | 100 | 99 | 100 | 99 | 93 |
| 101 | 50 | 100 | 100 | 99 | 100 | 99 | 99 |
| 102 | 50 | 100 | 100 | 100 | 100 | 98 | 100 |
| 103 | 50 | 100 | 100 | 100 | 97 | 91 | 99 |
| 104 | 50 | 100 | 100 | 100 | 100 | 97 | 99 |
| 105 | 50 | 100 | 99 | 99 | 79 | 96 | 99 |
| 106 | — | — | — | — | — | — | — |
| 107 | 50 | 100 | 100 | 100 | 100 | 97 | 100 |
| 108 | 50 | 100 | 100 | 100 | 70 | 98 | 99 |
| 109 | 50 | 100 | 74 | 99 | 0 | 96 | 85 |
| 110 | 50 | 100 | 100 | 100 | 79 | 96 | 99 |
| 111 | | — | — | — | — | — | — |
| 112 | | — | — | — | — | — | — |
| 113 | | — | — | — | — | — | — |
| 114 | | — | — | — | — | — | — |
| 115 | | — | — | — | — | — | — |
| 116 | 50 | 100 | 100 | 99 | 52 | 98 | 100 |
| 117 | 50 | 99 | 98 | 93 | 47 | 99 | 0 |
| 118 | 50 | 99 | 100 | 99 | 100 | 98 | 100 |
| 119 | 50 | 100 | 100 | 100 | 99 | 96 | 100 |
| 120 | 50 | 100 | 100 | 99 | 77 | 33 | 0 |
| 121 | 50 | 100 | 100 | 99 | 72 | 99 | 99 |
| 122 | 50 | 100 | 0 | 98 | 0 | 53 | 0 |
| 123 | 50 | 0 | 0 | 0 | 68 | 0 | 0 |
| 124 | 50 | 100 | 0 | 95 | 73 | 88 | 0 |
| 125 | 50 | 100 | 0 | 97 | 17 | 99 | 0 |
| 126 | 50 | 95 | 74 | 98 | 0 | 98 | 15 |
| 127 | | — | — | — | — | — | — |
| 128 | 50 | 100 | 100 | 100 | 99 | 98 | 100 |
| 129 | 50 | 100 | 100 | 100 | 67 | 99 | 100 |
| 130 | 50 | 100 | 100 | — | 77 | 98 | 98 |
| 131 | 50 | 96 | 74 | — | 19 | 97 | 0 |
| 132 | 50 | 100 | 68 | — | 0 | 97 | 0 |
| 133 | 50 | 3 | 0 | — | 0 | 0 | 0 |
| 134 | 50 | 100 | 100 | 100 | 96 | 98 | 100 |
| 135 | 50 | 100 | 100 | 100 | 100 | 97 | 100 |
| 136 | 50 | 99 | 100 | 100 | 97 | 95 | 40 |
| 137 | 50 | 100 | 100 | 100 | 100 | 66 | 100 |
| 138 | 50 | 100 | 100 | 100 | 100 | 93 | 100 |
| 139 | 50 | 100 | 86 | 91 | 0 | 93 | 9 |
| 140 | 50 | 99 | 100 | 99 | 100 | 99 | 100 |
| 141 | 50 | 99 | 98 | 99 | 0 | 98 | 99 |
| 142 | 50 | 95 | 68 | 99 | 0 | 98 | 98 |
| 143 | 250 | 6 | 80 | 100 | 0 | 0 | 0 |
| 144 | 250 | 0 | 0 | 0 | 74 | 0 | 0 |
| 145 | 50 | 100 | 100 | 100 | 99 | 100 | 100 |
| 146 | 50 | 100 | 100 | 100 | 96 | 98 | 99 |
| 147 | | — | — | — | — | — | — |
| 148 | | — | — | — | — | — | — |
| 149 | | — | — | — | — | — | — |
| 150 | | — | — | — | — | — | — |
| 151 | | — | — | — | — | — | — |
| 152 | | — | — | — | — | — | — |
| 153 | 250 | 0 | 0 | 96 | 71 | 0 | 0 |
| 154 | 50 | 100 | 100 | 100 | 87 | 100 | 100 |
| 155 | 50 | 99 | 100 | 100 | 100 | 100 | 100 |
| 156 | 50 | 99 | 100 | 99 | 96 | 100 | 87 |
| 157 | 50 | 98 | 100 | 100 | 100 | 99 | 100 |
| 158 | 50 | 100 | 100 | 100 | 100 | 99 | 99 |
| 159 | 50 | 100 | 100 | 99 | 100 | 99 | 100 |
| 160 | 50 | 100 | 100 | 100 | 73 | 99 | 0 |
| 161 | 50 | 98 | 99 | 100 | 99 | 96 | 47 |
| 162 | 50 | 100 | 89 | 98 | 13 | 99 | 16 |
| 163 | 50 | 100 | 100 | 78 | 77 | 100 | 99 |
| 164 | 50 | 100 | 100 | 99 | 100 | 99 | 100 |
| 165 | 50 | 100 | 100 | 99 | 92 | 95 | 100 |
| 166 | 50 | 100 | 91 | 79 | 0 | 95 | 67 |
| 167 | 50 | 100 | 100 | 100 | 0 | 86 | 100 |
| 168 | 50 | 100 | 96 | 72 | 0 | 81 | 0 |
| 169 | 50 | 37 | 68 | 43 | 0 | 93 | 0 |
| 170 | 50 | 99 | 89 | 76 | 0 | 51 | 80 |
| 171 | 50 | 99 | 99 | 99 | 0 | 100 | 96 |
| 172 | 250 | 79 | 92 | 87 | 78 | 83 | 0 |
| 173 | 50 | — | 100 | 94 | 84 | 92 | 99 |
| 174 | 50 | — | 95 | 97 | 30 | 83 | 91 |
| 175 | 50 | 100 | 100 | 99 | 100 | 98 | 100 |
| 176 | 250 | 4 | 80 | 0 | 0 | 0 | 0 |
| 177 | 50 | 88 | 80 | 0 | 0 | 0 | 0 |
| 178 | 50 | 100 | 100 | 100 | 100 | 93 | 100 |
| 179 | 50 | 100 | 100 | 100 | 100 | 94 | 100 |
| 180 | 50 | 100 | 100 | 100 | 100 | 99 | 100 |
| 181 | 50 | 98 | 100 | 100 | 100 | 94 | 100 |
| 182 | 50 | 0 | 0 | 0 | 0 | 0 | 0 |
| 183 | 50 | 100 | 100 | 96 | 94 | 98 | 100 |
| 184 | 50 | 100 | 100 | 96 | 99 | 96 | 99 |
| 185 | 50 | 100 | 100 | 99 | 100 | 96 | 100 |
| 186 | 50 | 99 | 100 | 97 | 99 | 90 | 100 |
| 187 | | — | — | — | — | — | — |
| 188 | | — | — | — | — | — | — |
| 189 | | — | — | — | — | — | — |
| 190 | | — | — | — | — | — | — |
| 191 | 250 | 72 | 9 | 99 | 0 | 99 | 0 |
| 192 | 250 | 38 | 80 | 84 | 91 | 93 | 0 |
| 193 | 250 | 97 | 100 | 100 | 61 | 100 | 99 |
| 194 | 50 | 100 | 100 | 100 | 100 | 100 | 99 |
| 195 | | — | — | — | — | — | — |
| 196 | | — | — | — | — | — | — |
| 197 | | — | — | — | — | — | — |
| 198 | | — | — | — | — | — | — |
| 199 | | — | — | — | — | — | — |
| 200 | | — | — | — | — | — | — |
| 201 | | — | — | — | — | — | — |
| 202 | 50 | 99 | 68 | 72 | 44 | 99 | 0 |
| 203 | 50 | 0 | 0 | 0 | 0 | 31 | 0 |
| 204 | 50 | 2 | 0 | 0 | 39 | 0 | 0 |
| 205 | 50 | 100 | 99 | 98 | 71 | 99 | 99 |
| 206 | 50 | 97 | 100 | 93 | 65 | 100 | 76 |
| 207 | 50 | 79 | 68 | 90 | 0 | 100 | 0 |
| 208 | 50 | 96 | 100 | 99 | 0 | 100 | 100 |

What is claimed is:

1. A compound selected from Formula 1, tautomers, N-oxides, and salts thereof, 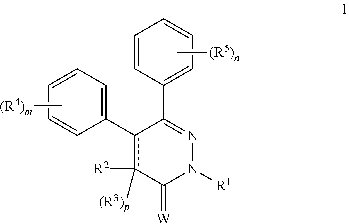
wherein
W is O or S;
R¹ is C₁-C₆ alkyl, C₂-C₆ alkenyl, C₂-C₆ alkynyl, C₃-C₆ cycloalkyl, C₂-C₆ cyanoalkyl or C₂-C₆ alkoxyalkyl, each optionally substituted with up to 3 substituents independently selected from halogen;
R² is H, halogen, cyano, hydroxy, nitro, C(═O)NR^{7a}R^{7b}, C(═O)OH, C₁-C₆ alkyl, C₁-C₆ haloalkyl, C₂-C₆ alkenyl C₂-C₆ haloalkenyl, C₂-C₆ alkynyl, C₂-C₆ haloalkynyl, C₃-C₆ cycloalkyl, C₃-C₆ halocycloalkyl, C₂-C₆ cyanoalkyl, C₁-C₆ hydroxyalkyl, C₂-C₆ alkoxyalkyl, C₂-C₆ haloalkoxyalkyl, C₁-C₆ alkoxy, C₁-C₆ haloalkoxy, C₂-C₆ alkenyloxy, C₂-C₆ haloalkenyloxy, C₂-C₆ alkynyloxy, C₂-C₆ haloalkynyloxy, C₃-C₆ cycloalkoxy, C₂-C₆ alkylcarbonyloxy, C₂-C₆ haloalkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₂-C₆ alkylcarbonyl, C₂-C₆ haloalkylcarbonyl, C₂-C₆ alkoxycarbonyl, C₂-C₆ haloalkoxycarbonyl, C₁-C₆ alkylamino, C₁-C₆ haloalkylamino or C₂-C₆ dialkylamino;
p is 0 or 1;
the dotted line in Formula 1 represents an optional bond, provided that the optional bond is present when p is 0, and the optional bond is absent when p is 1;
R³ is H or C₁-C₃ alkyl;
each R⁴ and R⁵ is independently cyano, nitro, halogen or hydroxy; or C₁-C₆ alkyl, C₂-C₆ alkenyl, C₂-C₆ alkynyl, C₂-C₆ cyanoalkyl, C₁-C₆ hydroxyalkyl, C₁-C₆ alkoxy, C₂-C₆ alkenyloxy, C₂-C₆ alkynyloxy, C₂-C₆ cyanoalkoxy or C₁-C₆ alkylthio, each optionally substituted with up to 3 substituents independently selected from halogen and C₁-C₃ alkyl; or —U—V-T;
each U is independently a direct bond, O, C(═O) or NR⁶;
each V is independently C₁-C₆ alkylene, C₂-C₆ alkenylene or C₃-C₆ alkynylene, wherein up to 2 carbon atoms are C(═O), each optionally substituted with up to 3 substituents independently selected from halogen, cyano, nitro, hydroxy, C₁-C₆ alkyl, C₁-C₆ haloalkyl, C₁-C₆ alkoxy and C₁-C₆ haloalkoxy;
each T is independently NR^{7a}R^{7b}, OR⁸ or S(═O)_{q}R⁹;
each R⁶ is independently H, C₁-C₆ alkyl, C₁-C₆ haloalkyl, C₂-C₆ alkylcarbonyl, C₂-C₆ haloalkylcarbonyl, C₂-C₆ alkoxycarbonyl, C₂-C₆ (alkylthio)carbonyl or C₂-C₆ alkoxy(thiocarbonyl);
each R^{7a} and R^{7b} is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₁-C₆ alkoxy, C₁-C₆ haloalkoxy, C₂-C₆ alkylcarbonyl or C₂-C₆ alkoxycarbonyl; or
R^{7a} and R^{7b} are taken together with the nitrogen atom to which they are attached to form a 3- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹⁰;
each R⁸ and R⁹ is independently H, C₁-C₆ alkyl, C₁-C₆ haloalkyl, C₂-C₆ alkenyl, C₂-C₆ haloalkenyl, C₂-C₆ alkynyl, C₃-C₆ cycloalkyl, C₃-C₆ halocycloalkyl, C₂-C₆ alkylcarbonyl, C₂-C₆ haloalkylcarbonyl or C₂-C₆ alkoxycarbonyl;
each R¹⁰ is independently halogen, C₁-C₃ alkyl, C₁-C₃ haloalkyl, C₁-C₃ alkoxy or C₁-C₃ haloalkoxy; and
m and n are each independently 0 to 5;
each q is independently 0, 1 or 2.
provided that the compound of Formula 1 is not:
2-methyl-5,6-diphenyl-3(2H)-pyridazinone;
2-ethyl-5,6-diphenyl-3(2H)-pyridazinone;
2-(methoxymethyl)-5,6-diphenyl-3(2H)-pyridazinone;
2-(2-methoxyethyl)-5,6-diphenyl-3(2H)-pyridazinone;
2-(2-methoxyethyl)-5,6-diphenyl-3(2H)-pyridazinethione;
2,3-dihydro-2-methyl-3-oxo-5,6-diphenyl-4-pyridazinecarbonitrile;
2,3-dihydro-3-oxo-5,6-diphenyl-2-propyl-4-pyridazinecarbonitrile;
2,3-dihydro-2-(1-methylethyl)-3-oxo-5,6-diphenyl-4-pyridazinecarbonitrile;
5-cyano-6-oxo-3,4-diphenyl-1(6H)-pyridazinepropanenitrile;
2,3-dihydro-3-oxo-2-(2-pentyn-1-yl)-5,6-diphenyl-4-pyridazinecarbonitrile;
5,6-bis(4-chlorophenyl)-2-methyl-3(2H)-pyridazinone;
2-(methoxymethyl)-5-(4-methylphenyl)-6-phenyl-3 (2H)-pyridazinone;
5-(4-chlorophenyl)-2-(methoxymethyl)-6-phenyl-3(2H)-pyridazinone;
2-(2-chloroethyl)-5,6-bis(4-chlorophenyl)-2,3-dihydro-3-oxo-4-pyridazinecarbonitrile;
5,6-bis(4-methoxyphenyl)-2-methyl-3(2H)-pyridazinone;
2-ethyl-5,6-bis(4-methoxyphenyl)-3(2H)-pyridazinone;
5,6-bis(4-methoxyphenyl)-2-(1-methylethyl)-3(2H)-pyridazinone;
2-cyclopropyl-5,6-bis(4-methoxyphenyl)-3(2H)-pyridazinone;
2-(2-chloroethyl)-5,6-bis(4-methoxyphenyl)-3(2H)-pyridazinone;
5,6-bis(4-methoxyphenyl)-2-(2-propen-1-yl)-3 (2H)-pyridazinone;
2-cyclopentyl-5,6-bis(4-methoxyphenyl)-3(2H)-pyridazinone;
2-ethyl-2,3-dihydro-5,6-bis(4-methoxyphenyl)-3-oxo-4-pyridazinecarbonitrile;
2,3-dihydro-5,6-bis(4-methoxyphenyl)-3-oxo-2-propyl-4-pyridazinecarbonitrile;
2,3-dihydro-5,6-bis(4-methoxyphenyl)-2-(1-methylethyl)-3-oxo-4-pyridazinecarbonitrile;
2-ethyl-6-(3-fluoro-4-methoxyphenyl)-5-(4-methoxyphenyl)-3(2H)-pyridazinone;
2-ethyl-5-(3-fluoro-4-methoxyphenyl)-6-(4-methoxyphenyl)-3(2H)-pyridazinone;
2-ethyl-5,6-bis(3-fluoro-4-methoxyphenyl)-3(2H)-pyridazinone;
2-ethyl-5,6-bis(3-fluoro-4-methoxyphenyl)-4,5-dihydro-3(2H)-pyridazinone;
6-(4-methoxyphenyl)-2-methyl-5-(3,4,5-trimethoxyphenyl)-3(2H)-pyridazinone;
2-methyl-4-nitro-5,6-diphenyl-3(2H)-pyridazinone;
2-methyl-4-(methylthio)-5,6-diphenyl-3(2H)-pyridazinone; and
4-(ethylthio)-2-methyl-5,6-diphenyl-3(2H)-pyridazinone.

2. A compound claim 1 wherein
W is O;
R¹ is C₁-C₃ alkyl, C₂-C₅ alkenyl, C₂-C₅ alkynyl, C₃-C₅ cycloalkyl, C₂-C₄ cyanoalkyl or C₂-C₅ alkoxyalkyl;
R² is H, halogen, cyano, C₁-C₃ alkyl, C₁-C₃ haloalkyl, cyclopropyl, halocyclopropyl, C₁-C₃ alkoxy, C₁-C₃ haloalkoxy, C₂-C₃ alkenyloxy or C₂-C₃ haloalkenyloxy;
R³ is H, methyl or ethyl;
each R⁴ and R⁵ is independently cyano, nitro or halogen; or C₁-C₃ alkyl, C₂-C₃ alkenyl, C₂-C₄ cyanoalkyl, C₁-C₃ alkoxy, C₂-C₄ alkenyloxy or C₂-C₄ cyanoalkoxy, each optionally substituted with up to 3 substituents independently selected from halogen; or —U—V-T;
each U is independently a direct bond, O, C(═O) or NH;
each V is independently C₁-C₃ alkylene, wherein up to 1 carbon atom is C(═O), each optionally substituted with up to 2 substituents independently selected from halogen, methyl, halomethyl and methoxy;
each T is independently NR^{7a}R^{7b} or ORB;
each R^{7a} and R^{7b} is independently H, C₁-C₃ alkyl, C₁-C₃ haloalkyl, cyclopropyl, C₂-C₃ alkylcarbonyl or C₂-C₃ alkoxycarbonyl; and
m and n are each independently 1 to 4.

3. A compound of claim 2 wherein
R¹ is C₁-C₂ alkyl, C₃-C₄ cycloalkyl or C₂-C₃ cyanoalkyl;
R² is H, halogen, cyano, C₁-C₂ alkyl, C₁-C₂ haloalkyl, C₁-C₂ alkoxy or C₁-C₂ haloalkoxy;
R³ is H or methyl;
each R⁴ and R⁵ is independently cyano or halogen; or C₁-C₂ alkyl or C₁-C₂ alkoxy, each optionally substituted with up to 3 substituents independently selected from halogen; or —U—V-T;
each U is independently a direct bond, O or NH;
each V is independently CH₂, CH₂CH₂ or C(═O);
each R^{7a} and R^{7b} is independently H, C₁-C₂ alkyl, C₁-C₂ haloalkyl or cyclopropyl;
m and n are each independently 1 to 3.

4. A compound of claim 3 wherein
R¹ is methyl, ethyl, cyclopropyl or —CH₂C≡N;
R² is H, halogen, C₁-C₂ alkyl or methoxy;
p is 0; and
each R⁴ and R⁵ is independently halogen or methoxy.

5. A compound of claim 4 wherein
R¹ is methyl;
R² is Br, Cl, methyl, ethyl or methoxy;
each R⁴ and R⁵ is independently Br, Cl, F or methoxy;
m is 2 and the R⁴ substituents are attached at the 2- and 6-positions; or m is 2 and the R⁴ substituents are attached at the 2- and 4-positions; or m is 2 and the R⁴ substituents are attached at the 3- and 5-positions; and
n is 2 and the R⁵ substituents are attached at the 3- and 5-positions; or n is 2 and the R⁵ substituents are attached at the 2- and 4-positions; or n is 2 and the R⁵ substituents are attached at the 2- and 5-positions; or n is 2 and the R⁵ substituents are attached at the 2- and 6-positions; or n is 3 and the R⁵ substituents are attached at the 2-, 3- and 5-positions.

6. A compound of claim 5 wherein
R² is C₁, methyl, ethyl or methoxy;
each R⁴ is independently Cl or F; and
each R⁵ is independently Br, Cl, F or methoxy.

7. A compound of claim 6 wherein
R² is Cl or methyl;
each R⁵ is independently Cl, F or methoxy.

8. A compound of claim 1 which is selected from the group:
6-(2-chloro-3,5-dimethoxyphenyl)-5-(2-chloro-4-fluorophenyl)-2-methyl-3(2H)-pyridazinone;
4-chloro-6-(2-chloro-3,5-dimethoxyphenyl)-5-(2,6-difluorophenyl)-2-methyl-3(2H)-pyridazinone;
5,6-bis(2-chloro-4-fluorophenyl)-2,4-dimethyl-3(2H)-pyridazinone;
4-chloro-6-(2-chloro-5-methoxyphenyl)-5-(2,6-difluorophenyl)-2-methyl-3(2H)-pyridazinone;
4-chloro-6-(2-chloro-3,5-dimethoxyphenyl)-5-(2-chloro-4-fluorophenyl)-2-methyl-3(2H)-pyridazinone;
6-(2-bromo-3,5-dimethoxyphenyl)-5-(2-chloro-4-fluorophenyl)-2,4-dimethyl-3(2H)-pyridazinone;
6-(2-chloro-3,5-dimethoxyphenyl)-5-(2-chloro-4-fluorophenyl)-2,4-dimethyl-3(2H)-pyridazinone;
6-(2-chloro-5-methoxyphenyl)-5-(2,6-difluorophenyl)-2,4-dimethyl-3(2H)-pyridazinone;
6-(2-bromo-3,5-dimethoxyphenyl)-4-chloro-5-(2-chloro-4-fluorophenyl)-2-methyl-3(2H)-pyridazinone;
6-(2-chloro-3,5-dimethoxyphenyl)-5-(2-chloro-4-fluorophenyl)-4-methoxy-2-methyl-3(2H)-pyridazinone;
4-chloro-5-(2-chloro-4-fluorophenyl)-6-(2-chloro-5-methoxyphenyl)-2-methyl-3(2H)-pyridazinone;
5-(2-chloro-4-fluorophenyl)-6-(2-chloro-5-methoxyphenyl)-2,4-dimethyl-3(2H)-pyridazinone;
5-(2-chloro-4-fluorophenyl)-6-(2-chloro-5-methoxyphenyl)-4-ethyl-2-methyl-3(2H)-pyridazinone;
6-(2-chloro-5-methoxyphenyl)-5-(2,6-difluorophenyl)-4-ethyl-2-methyl-3(2H)-pyridazinone; and
6-(2-chloro-3,5-dimethoxyphenyl)-5-(2,6-difluorophenyl)-2,4-dimethyl-3(2H)-pyridazinone.

9. A fungicidal composition comprising (a) a compound of claim 1; and (b) at least one other fungicide.

10. A fungicidal composition comprising (a) a compound of claim 1; and (b) at least one additional component selected from the group consisting of surfactants, solid diluents and liquid diluents.

11. A method for controlling plant diseases caused by fungal plant pathogens comprising applying to the plant or portion thereof, or to the plant seed, a fungicidally effective amount of a compound of claim 1.
